SEQUENCE LISTING (27 sequences)

SEQ ID NO: 1 - RNA, 260 nt, Unknown
  Description of Unknown Organism  GB VIRUS B
gaguuugcgaccaugguggaucagaaccguuucgggugaagccauggucugaaggggaugacgucccuucuggcucauccacaaaaaccgucucgggugggugaggaguccuggcugugugggaagcagucaguauaauucccgucguguguggugacgccucacgacguauuuguccgcugugcagagcguaguaccaagggcugcaccccgguuuuuguuccaagcggagggcaacccccgcuuggaauuaaaaacug

SEQ ID NO: 2 - DNA, 9399 bp, GBV-A-like virus
accacaaacactccagtttgttacactccgctaggaatgctcctggagcaccccccctagcagggcgtgggggatttcccctgcccgtctgcagaagggtggagccaaccaccttagtatgtaggcggcgggactcatgacgctcgcgtgatgacaagcgccaagcttgacttggatggccctgatgggcgttcatgggttcggtggtggtggcgctttaggcagcctccacgcccaccacctcccagatagagcggcggcactgtagggaagaccggggaccggtcactaccaaggacgcagacctctttttgagtatcacgcctccggaagtagttgggcaagcccacctatatgtgttgggatggttggggttagccatccataccgtactgcctgatagggtccttgcgaggggatctgggagtctcgtagaccgtagcacatgcctgttatttctactcaaacaagtcctgtacctgcgcccagaacgcgcaagaacaagcagacgcaggcttcatatcctgtgtccattaaaacatctgttgaaaggggacaacgagcaaagcgcaaagtccagcgcgatgctcggcctcgtaattacaaaattgctggtatccatgatggcttgcagacattggctcaggctgctttgccagctcatggttggggacgccaagaccctcgccataagtctcgcaatcttggaatccttctggattaccctttggggtggattggtgatgttacaactcacacacctctagtaggcccgctggtggcaggagcggtcgttcgaccagtctgccagatagtacgcttgctggaggatggagtcaactgggctactggttggttcggtgtccacctttttgtggtatgtctgctatctttggcctgtccctgtagtggggcgcgggtcactgacccagacacaaataccacaatcctgaccaattgctgccagcgtaatcaggttatctattgttctccttccacttgcctacacgagcctggttgtgtgatctgtgcggacgagtgctgggttcccgccaatccgtacatctcacacccttccaattggactggcacggactccttcttggctgaccacattgattttgttatgggcgctcttgtgacctgtgacgcccttgacattggtgagttgtgtggtgcgtgtgtattagtcggtgactggcttgtcaggcactggcttattcacatagacctcaatgaaactggtacttgttacctggaagtgcccactggaatagatcctgggttcctagggtttatcgggtggatggccggcaaggtcgaggctgtcatcttcttgaccaaactggcttcacaagtaccatacgctattgcgactatgtttagcagtgtacactacctggcggttggcgctctgatctactatgcctctcggggcaagtggtatcagttgctcctagcgcttatgctttacatagaagcgacctctggaaaccctatcagggtgcccactggatgctcaatagctgagttttgctcgcctttgatgataccatgtccttgccactcttatttgagtgagaatgtgtcagaagtcatttgttacagtccaaagtggaccaggcctatcactctagagtataacaactccatatcttggtacccctatacaatccctggtgcgaggggatgtatggttaaattcaaaaataacacatggggttgctgccgtattcgcaatgtgccatcgtactgcactatgggcactgatgcagtgtggaacgacactcgcaacacttacgaagtatgcggtgtaacaccatggctaacaaccgcatggcacaacggctcagccctgaaattggctatattacaataccctgggtctaaagaaatgtttaaacctcataattggatgtcaggccatttgtattttgagggatcagatacccctatagtttacttttatgaccctgtgaattccactctcctaccaccggagaggtgggctaggttgcccggtaccccacctgtggtacgtggttcttggttacaggttccgcaagggttttacagtgatgtgaaagacctagccacaggattgatcaccaaagacaaagcctggaaaaattatcaggtcttatattccgccacgggtgctttgtctcttacgggagttaccaccaaggccgtggtgctaattctgttggggttgtgtggcagcaagtatcttattttagcctacctctgttacttgtccctttgttttgggcgcgcttctggttaccctttgcgtcctgtgctcccatcccagtcgtatctccaagctggctgggatgttttgtctaaagctcaagtagctccttttgctttgattttcttcatctgttgctatctccgctgcaggctacgttatgctgcccttttagggtttgtgcccatggctgcgggcttgcccctaactttctttgttgcagcagctgctgcccaaccagattatgactggtgggtgcgactgctagtggcagggttagttttgtgggccggccgtaaccgtggtcaccgcatagctctgcttgtaggtccttggcctctggtagcgcttttaaccctcttgcatttggttacgcctgcttcagcttttgataccgagataattggagggctgacaataccacctgtagtagcattagttgtcatgtctcgttttggcttctttgctcacttgttacctcgctgtgctttagttaactcctatctttggcaacgttgggagaattggttttggaacgttacactaagaccggagaggtttttccttgtgctggtttgtttccccggtgcgacatatgacgcgctggtgactttctgtgtgtgtcacgtagctcttctatgtttaacatccagtgcagcatcgttctttgggactgactctagggttagggcccatagaatgttggtgcgtctcggaaagtgtcatgcttggtattctcattatgttcttaagtttttcctcttagtgtttggtgagaatggtgtgtttttctataagcacttgcatggtgatgtcttgcctaatgattttgcctcgaaactaccattgcaagagccatttttcccttttgaaggcaaggcaagggtctataggaatgaaggaagacgcttggcgtgtggggacacggttgatggtttgcccgttgttgcgcgtctcggcgaccttgttttcgcagggttggctatgccgccagatgggtgggccattaccgcaccttttacgctgcagtgtctctctgaacgtggcacgctgtcagcgatggcagtggtcatgactggtatagacccccgaacttggactggaactatcttcagattaggatctctggccactagctacatgggatttgtttgtgacaacgtgttgtatactgctcaccatggcagcaaggggcgccggttggctcatcccacaggctctatacacccaataaccgttgacgcggctaatgaccaggacatctatcaaccaccatgtggagctgggtcccttactcggtgctcttgcggggagaccaaggggtatctggtaacacgactggggtcattggttgaggtcaacaaatccgatgacccttattggtgtgtgtgcggggcccttcccatggctgttgccaagggttcttcaggtgccccgattctgtgctcctccgggcatgttattgggatgttcaccgctgctagaaattctggcggttcagtcagtcagattagggttaggccgttggtgtgtgctggataccatccccagtacacagcacatgccactcttgatacaaaacctactgtgcctaacgagtattcagtgcaaattttaattgcccccactggcagcggcaagtcaaccaaattaccactttcttacatgcaggagaagtatgaggtcttggtcctaaatcccagtgtggctacaacagcatcaatgccaaagtacatgcacgcgacgtacggcgtgaatccaaattgctattttaatggcaaatgtaccaacacaggggcttcacttacgtacagcacatatggcatgtacctgaccggagcatgttcccggaactatgatgtaatcatttgtgacgaatgccatgctaccgatgcaaccaccgtgttgggcattggaaaggtcctaaccgaagctccatccaaaaatgttaggctagtggttcttgccacggctaccccccctggagtaatccctacaccacatgccaacataactgagattcaattaaccgatgaaggcactatcccctttcatggaaaaaagattaaggaggaaaatctgaagaaagggagacaccttatctttgaggctaccaaaaaacactgtgatgagcttgctaacgagttagctcgaaagggaataacagctgtctcttactataggggatgtgacatctcaaaaatccctgagggcgactgtgtagtagttgccactgatgccttgtgtacagggtacactggtgactttgattccgtgtatgactgcagcctcatggtagaaggcacatgccatgttgaccttgaccctactttcaccatgggtgttcgtgtgtgcggggtttcagcaatagttaaaggccagcgtaggggccgcacaggccgtgggagagctggcatatactactatgtagacgggagttgtaccccttcgggtatggttcctgaatgcaacattgttgaagccttcgacgcagccaaggcatggtatggtttgtcatcaacagaagctcaaactattctggacacctatcgcacccaacctgggttacctgcgataggagcaaatttggacgagtgggctgatctcttttctatggtcaaccccgaaccttcatttgtcaatactgcaaaaagaactgctgacaattatgttttgttgactgcagcccaactacaactgtgtcatcagtatggctatgctgctcccaatgacgcaccacggtggcagggagcccggcttgggaaaaaaccttgtggggttctgtggcgcttggacggcgctgacgcctgtcctggcccagagcccagcgaggtgaccagataccaaatgtgcttcactgaagtcaatacttctgggacagccgcactcgctgttggcgttggagtggctatggcttatctagccattgacacttttggcgccacttgtgtgcggcgttgctggtctattgcatcagtccctaccggtgctactgtcgccccagtggttgacgaagaagaaatcgtggaggagtgtgcatcattcattcccttggaggccatggttgctgcaatcgataagctgaagagtacaatcaccacaactagtcctttcacattggaaaccgcccttgaaaaacttaacacctttcttgggcctcatgcagctacaatccttgctatcatagagtattgctgtggtttagtcactttacctgacaatccctttgcatcatgcgtgtttgctttcattgcgggtattactaccccactacctcacaagatcaaaatgttcctgtcattatttggaggcgcaattgcgtccaagcttacagacgctagaggcgcactggcgttcatgatggccggggctgcgggaacagctcttggtacatggacatcggtgggttttgtctttgacatgctaggcggctatgctgccgcctcatccactgcttgcttgacatttaaatgcttgatgggtgagtggcccactatggatcagcttgctggtttagtctactccgcgttcaatccggccgcaggagttgtgggcgtcttgtcagcttgtgcaatgtttgctttgacaacagcagggccagatcactggcccaacagacttcttactatgcttgctaggagcaacactgtatgtaatgagtactttattgccactcgtgacatccgcaggaagatactgggcattctggaggcatctaccccctggagtgtcatatcagcttgcatccgttggctccacaccccgacggaggatgattgcggcctcattgcttggggtctagagatttggcagtatgtgtgcaatttctttgtgatttgctttaatgtccttaaagctggagttcagagcatggttaacattcctggttgtcctttctacagctgccagaaggggtacaagggcccctggattggatcaggtatgctccaagcacgctgtccatgcggtgctgaactcatcttttctgttgagaatggttttgcaaaactttacaaaggacccagaacttgttcaaattactggagaggggctgttccagtcaacgctaggctgtgtgggtcggctagaccggacccaactgattggactagtcttgtcgtcaattatggcgttagggactactgtaaatatgagaaaatgggagatcacatttttgttacagcagtatcctctccaaatgtctgtttcacccaggtgcccccaaccttgagagctgcagtggccgtggacggcgtacaggttcagtgttatctaggtgagcccaaaactccttggacgacatctgcttgctgttacggtcctgacggtaagggtaaaactgttaagcttcccttccgcgttgacggtcacacacctggtgtgcgcatgcaacttaatttgcgtgatgcacttgagacaaatgactgtaattccacaaacaacactcctagtgatgaagccgcagtgtccgctcttgttttcaaacaggagttgcggcgtacaaaccaattgcttgaggcaatttcagctggcgttgacaccaccaaactgccagccccctccatcgaagaggtagtggtaagaaagcgccagttccgggcaagaactggttcgcttaccttgcctccccctccgagatccgtcccaggagtgtcatgtcctgaaagcctgcaacgaagtgacccgttagaaggtccttcaaacctccctccttcaccacctgttctacagttggccatgccgatgcccctgttgggagcgggtgagtgtaaccctttcactgcaattggatgtgcaatgaccgaaacaggcggaggccctgatgatttacccagttaccctcccaaaaaggaggtctctgaatggtcagacgaaagttggtcgacggctacaaccgtttccagctacgttactggccccccgtaccctaagatacggggaaaggattccactcagtcagcccccgccaaacggcctacaaaaaagaagttgggaaagagtgagttttcgtgcagcatgagctacacctggaccgacgtgattagcttcaaaactgcttctaaagttctgtctgcaactcgggccatcactagtggtttcctcaaacaaagatcattggtgtatgtgactgagccgcgggatgcggagcttagaaaacaaaaagtcactattaatagacaacctctgttccccccatcataccacaagcaagtgagattggctaaggaaaaagcttcaaaagttgtcggtgtcatgtgggactatgatgaagtagcagctcacacgccctctaagtctgctaagtcccacatcactggccttcggggcactgatgttcgttctggagcagcccgcaaggctgttctggacttgcagaagtgtgtcgaggcaggtgagataccgagtcattatcggcaaactgtgatagttccaaaggaggaggtcttcgtgaagaccccccagaaaccaacaaagaaacccccaaggcttatctcgtacccccaccttgaaatgagatgtgttgagaagatgtactacggtcaggttgctcctgacgtagttaaagctgtcatgggagatgcgtacgggtttgtagatccacgtacccgtgtcaagcgtctgttgtcgatgtggtcacccgatgcagtcggagccacatgcgatacagtgtgttttgacagtaccatcacacccgaggatatcatggtggagacagacatctactcagcagctaaactcagtgaccaacaccgagctggcattcacaccattgcgaggcagttatacgctggaggaccgatgatcgcttatgatggccgagagatcggatatcgtaggtgtaggtcttccggcgtctatactacctcaagttccaacagtttgacctgctggctgaaggtaaatgctgcagccgaacaggctggcatgaagaaccctcgcttccttatttgcggcgatgattgcaccgtaatttggaagagcgccggagcagatgcagacaaacaagcaatgcgtgtctttgctagctggatgaaggtgatgggtgcaccacaagattgtgtgcctcaacccaaatacagtttggaagaattaacatcatgctcatcaaatgttacctctggaattaccaaaagtggcaagccttactactttcttacaagagatcctcgtatcccccttggcaggtgctctgccgagggtctgggatacaaccccagtgctgcgtggattgggtatctaatacatcactacccatgtttgtgggttagccgtgtgttggctgtccatttcatggagcagatgctctttgaggacaaacttcccgagacggtgacctttgactggtatgggaaaaattatacggtgcctgtagaagatctgcccagcatcattgctggtgtgcacggtattgaggctttctcggtggtgcgctacaccaacgctgagatcctcagagtttcccaatcactaacagacatgaccatgccccccctgcgagcctggcgaaagaaagccagggcggtcctcgccagcgccaagaggcgtggcggagcacacgcaaaattggctcgcttccttctctggcatgctacatctagacctctaccagatttggataagacgagcgtggctcggtacaccactttcaattattgtgatgtttactccccggagggggatgtgtttattacaccacagagaagattgcagaagtttcttgtgaagtatttggctgtcattgtttttgccctagggctcattgctgttggattagccatcagctgaacccccaaattcaaaattaactaacagtttttttttttttttttttttttagggcagcggcaacaggggagaccccgggcttaacgaccccgccgatgtgagtttggcgaccatggtggatcagaaccgtttcgggtgaagccatggtctgaaggggatgacgtcccttctggctcatccacaaaaaccgtctcgggtgggtgaggagtcctggctgtgtgggaagcagtcagtataattcccgtcgtgtgtggtgacgcctcacgacgtatttgtccgctgtgcagagcgtagtaccaagggctgcaccccggtttttgttccaagcggagggcaacccccgcttggaattaaaaact

SEQ ID NO: 3 - DNA, 48 bp, Artificial Sequence
  Description of Artificial Sequence  Primer
cgggatcccgtaatacgactcactatagaccacaaacactccagtttg

SEQ ID NO: 4 - DNA, 20 bp, Artificial Sequence
  Description of Artificial Sequence  Primer
gtggaattcacagcgtcata

SEQ ID NO: 5 - DNA, 21 bp, Artificial Sequence
  Description of Artificial Sequence  Primer
tgtgaattccactctcctacc

SEQ ID NO: 6 - DNA, 21 bp, Artificial Sequence
  Description of Artificial Sequence  Primer
ttatcgattgcagcaaccatg

SEQ ID NO: 7 - DNA, 39 bp, Artificial Sequence
  Description of Artificial Sequence  Primer
catggttgctgcaatcgataagctgaagagtacaataac

SEQ ID NO: 8 - DNA, 20 bp, Artificial Sequence
  Description of Artificial Sequence  Primer
gacaacagacgcttgacacg

SEQ ID NO: 9 - DNA, 20 bp, Artificial Sequence
  Description of Artificial Sequence  Primer
ctgtcatgggagatgcgtac

SEQ ID NO: 10 - DNA, 42 bp, Artificial Sequence
  Description of Artificial Sequence  Primer
cgagctcgagcacatcgcggggtcgttaagcccggggtctcc

SEQ ID NO: 11 - DNA, 20 bp, Artificial Sequence
  Description of Artificial Sequence  Primer
gacaacagacgcttgacacg

SEQ ID NO: 12 - DNA, 108 bp, Artificial Sequence
  Description of Artificial Sequence  Primer
ccgactcgagaattcggccctgcaggccacaacagtctcgcgagtttttaattccaagcgggggttgccctccgcttggaacaaaaaccggggtgcagcccttggtac

SEQ ID NO: 13 - DNA, 9599 bp, Hepatitis C virus
  CDS (342)..(9377)
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagcctaccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaaatgccggccgcaccacggctgggcttgttggtctccttacaccaggcgccaagcagaacatccaactgatcaacaccaacggcagttggcacatcaatagcacggccttgaattgcaatgaaagccttaacaccggctggttagcagggctcttctatcaacacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgcttccgcaaacatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccgttgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttccttctgcttgcagacgcgcgcgtctgctcctgcttgtggatgatgttactcatatcccaagcggaggcggctttggagaacctcgtaatactcaatgcagcatccctggccgggacgcacggtcttgtgtccttcctcgtgttcttctgctttgcgtggtatctgaagggtaggtgggtgcccggagcggtctacgccctctacgggatgtggcctctcctcctgctcctgctggcgttgcctcagcgggcatacgcactggacacggaggtggccgcgtcgtgtggcggcgttgttcttgtcgggttaatggcgctgactctgtcgccatattacaagcgctatatcagctggtgcatgtggtggcttcagtattttctgaccagagtagaagcgcaactgcacgtgtgggttccccccctcaacgtccggggggggcgcgatgccgtcatcttactcatgtgtgtagtacacccgaccctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgtcttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgctggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctactgcctttgtgggtgctggcctagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggttggggtaaacactccggcctcttaagccatttcctgttttttttttttttttttttttttttttctttttttttttctttcctttccttctttttttcctttctttttcccttctttaatggtggctccatcttagccctagtcacggctagctgtgaaaggtccgtgagccgcatgactgcagagagtgctgatactggcctctctgcagatcatgt

SEQ ID NO: 14 - protein, 3011 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGNAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGRWVPGAVYALYGMWPLLLLLLALPQRAYALDTEVAASCGGVVLVGLMALTLSPYYKRYISWCMWWLQYFLTRVEAQLHVWVPPLNVRGGRDAVILLMCVVHPTLVFDITKLLLAIFGPLWILQASLLKVPYFVRVQGLLRICALARKIAGGHYVQMAIIKLGALTGTYVYNHLTPLRDWAHNGLRDLAVAVEPVVFSRMETKLITWGADTAACGDIINGLPVSARRGQEILLGPADGMVSKGWRLLAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLGTTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEECSQHLPYIEQGMMLAEQFKQKALGLLQTASRHAEVITPAVQTNWQKLEVFWAKHMWNFISGIQYLAGLSTLPGNPAIASLMAFTAAVTSPLTTGQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVAHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALNCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGRLDLSGWFTAGYSGGDIYHSVSHARPRWFWFCLLLLAAGVGIYLLPNR

SEQ ID NO: 15 - DNA, 21 bp, Artificial Sequence
  Description of Artificial Sequence  Synthetic Primer
gaaggagggaggtttgaagga

SEQ ID NO: 16 - DNA, 19 bp, Artificial Sequence
  Description of Artificial Sequence  Synthetic Primer
ccagttccgggcaagaact

SEQ ID NO: 17 - DNA, 13042 bp, Artificial Sequence
  Description of Artificial Sequence  Synthetic Primer
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgcctgttatttctactcaaacaagtcctgtacctgcgcccagaacgcgcaagaacaagcagacgcaggcttcatatcctgtgtccattaaaacatctgttgaaaggggacaacgagcaaagcgcaaagtccagcgcgatgctcggcctcgtaattacaaaattgctggtatccatgatggcttgcagacattggctcaggctgctttgccagctcatggttggggacgccaagaccctcgccataagtctcgcaatcttggaatccttctggattaccctttggggtggattggtgatgttacaactcacacacctctagtaggcccgctggtggcaggagcggtcgttcgaccagtctgccagatagtacgcttgctggaggatggagtcaactgggctactggttggttcggtgtccacctttttgtggtatgtctgctatctttggcctgtccctgtagtggggcgcgggtcactgacccagacacaaataccacaatcctgaccaattgctgccagcgtaatcaggttatctattgttctccttccacttgcctacacgagcctggttgtgtgatctgtgcggacgagtgctgggttcccgccaatccgtacatctcacacccttccaattggactggcacggactccttcttggctgaccacattgattttgttatgggcgctcttgtgacctgtgacgcccttgacattggtgagttgtgtggtgcgtgtgtattagtcggtgactggcttgtcaggcactggcttattcacatagacctcaatgaaactggtacttgttacctggaagtgcccactggaatagatcctgggttcctagggtttatcgggtggatggccggcaaggtcgaggctgtcatcttcttgaccaaactggcttcacaagtaccatacgctattgcgactatgtttagcagtgtacactacctggcggttggcgctctgatctactatgcctctcggggcaagtggtatcagttgctcctagcgcttatgctttacatagaagcgacctctggaaaccctatcagggtgcccactggatgctcaatagctgagttttgctcgcctttgatgataccatgtccttgccactcttatttgagtgagaatgtgtcagaagtcatttgttacagtccaaagtggaccaggcctatcactctggagtataacaactccatatcttggtacccctatacaatccctggtgcgaggggatgtatggttaaattcaaaaataacacatggggttgctgccgtattcgcaatgtgccatcgtactgcactatgggcactgatgcagtgtggaacgacactcgcaacacttacgaagtatgcggtgtaacaccatggctaacaaccgcatggcacaacggctcagccctgaaattggctatattacaataccctgggtctaaagaaatgtttaaacctcataattggatgtcaggccatttgtattttgagggatcagatacccctatagtttacttttatgaccctgtgaattccactctcctaccaccggagaggtgggctaggttgcccggtaccccacctgtggtacgtggttcttggttacaggttccgcaagggttttacagtgatgtgaaagacctagccacaggattgatcaccaaagacaaagcctggaaaaattatcaggtcttatattccgccacgggtgctttgtctcttacgggagttaccaccaaggccgtggtgctaattctgttggggttgtgtggcagcaagtatcttattttagcctacctctgttacttgtccctttgttttgggcgcgcttctggttaccctttgcgtcctgtgctcccatcccagtcgtatctccaagctggctgggatgttttgtctaaagctcaagtagctccttttgctttgattttcttcatctgttgctatctccgctgcaggctacgttatgctgcccttttagggtttgtgcccatggctgcgggcttgcccctaactttctttgttgcagcagctgctgcccaaccagattatgactggtgggtgcgactgctagtggcagggttagttttgtgggccggccgtaaccgtggtcaccgcatagctctgcttgtaggtccttggcctctggtagcgcttttaaccctcttgcatttggttacgcctgcttcagcttttgataccgagataattggagggctgacaataccacctgtagtagcattagttgtcatgtctcgttttggcttctttgctcacttgttacctcgctgtgctttagttaactcctatctttggcaacgttgggagaattggttttggaacgttacactaagaccggagaggtttttccttgtgctggtttgtttccccggtgcgacatatgacgcgctggtgactttctgtgtgtgtcacgtagctcttctatgtttaacatccagtgcagcatcgttctttgggactgactctagggttagggcccatagaatgttggtgcgtctcggaaagtgtcatgcttggtattctcattatgttcttaagtttttcctcttagtgtttggtgagaatggtgtgtttttctataagcacttgcatggtgatgtcttgcctaatgattttgcctcgaaactaccattgcaagagccatttttcccttttgaaggcaaggcaagggtctataggaatgaaggaagacgcttggcgtgtggggacacggttgatggtttgcccgttgttgcgcgtctcggcgaccttgttttcgcagggttggctatgccgccagatgggtgggccattaccgcaccttttacgctgcagtgtctctctgaacgtggcacgctgtcagcgatggcagtggtcatgactggtatagacccccgaacttggactggaactatcttcagattaggatctctggccactagctacatgggattcgtttgtgacaacgtgttgtatactgctcaccatggcagcaaggggcgccggttggctcatcccacaggctccatacacccaataaccgttgacgcggctaatgaccaggacatctatcaaccaccatgtggagctgggtcccttactcggtgctcttgcggggagaccaaggggtatctggtaacacgactggggtcattggttgaggtcaacaaatccgatgacccttattggtgtgtgtgcggggcccttcccatggctgttgccaagggttcttcaggtgccccgattctgtgctcctccgggcatgttattgggatgttcaccgctgctagaaattctggcggttcagtcagtcagattagggttaggccgttggtgtgtgctggataccatccccagtacacagcacatgccactcttgatacaaaaacgcgtatgcagatcttcgtgaagaccctgacgggcaagaccatcactcttgaggtcgagcccagtgacaccatcgagaatgtcaaggccaagatccaagacaaggaaggcatcccacctgaccagcagaggctgatattcgcgggcaaacagctggaggatggccgcaccctgtccgactacaacatccagaaagagtccaccttgcacctggtgctgcgtctccgcggtggcgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctactgcctttgtgggtgctggcctagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggttggggtaaacactccggcctcttaagccatttcctgttttttttttttttttttttttttttttctttttttttttctttcctttccttctttttttcctttctttttcccttctttaatggtggctccatcttagccctagtcacggctagctgtgaaaggtccgtgagccgcatgactgcagagagtgctgatactggcctctctgcagatcatgttctagactggaattcgtcgacgagctccctatagtgagtcgtattagaggccgacttggccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacaaaatattaacgtttacaatttcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacttggtcagcggccgctaatacgactcactata

SEQ ID NO: 18 - DNA, 13147 bp, Artificial Sequence
  Description of Artificial Sequence  Synthetic Primer
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagccgcgcgggtcactgacccagacacaaataccacaatcctgaccaattgctgccagcgtaatcaggttatctattgttctccttccacttgcctacacgagcctggttgtgtgatctgtgcggacgagtgctgggttcccgccaatccgtacatctcacacccttccaattggactggcacggactccttcttggctgaccacattgattttgttatgggcgctcttgtgacctgtgacgcccttgacattggtgagttgtgtggtgcgtgtgtattagtcggtgactggcttgtcaggcactggcttattcacatagacctcaatgaaactggtacttgttacctggaagtgcccactggaatagatcctgggttcctagggtttatcgggtggatggccggcaaggtcgaggctgtcatcttcttgaccaaactggcttcacaagtaccatacgctattgcgactatgtttagcagtgtacactacctggcggttggcgctctgatctactatgcctctcggggcaagtggtatcagttgctcctagcgcttatgctttacatagaagcgacctctggaaaccctatcagggtgcccactggatgctcaatagctgagttttgctcgcctttgatgataccatgtccttgccactcttatttgagtgagaatgtgtcagaagtcatttgttacagtccaaagtggaccaggcctatcactctagagtataacaactccatatcttggtacccctatacaatccctggtgcgaggggatgtatggttaaattcaaaaataacacatggggttgctgccgtattcgcaatgtgccatcgtactgcactatgggcactgatgcagtgtggaacgacactcgcaacacttacgaagtatgcggtgtaacaccatggctaacaaccgcatggcacaacggctcagccctgaaattggctatattacaataccctgggtctaaagaaatgtttaaacctcataattggatgtcaggccatttgtattttgagggatcagatacccctatagtttacttttatgaccctgtgaattccactctcctaccaccggagaggtgggctaggttgcccggtaccccacctgtggtacgtggttcttggttacaggttccgcaagggttttacagtgatgtgaaagacctagccacaggattgatcaccaaagacaaagcctggaaaaattatcaggtcttatattccgccacgggtgctttgtctcttacgggagttaccaccaaggccgtggtgctaattctgttggggttgtgtggcagcaagtatcttattttagcctacctctgttacttgtccctttgttttgggcgcgcttctggttaccctttgcgtcctgtgctcccatcccagtcgtatctccaagctggctgggatgttttgtctaaagctcaagtagctccttttgctttgattttcttcatctgttgctatctccgctgcaggctacgttatgctgcccttttagggtttgtgcccatggctgcgggcttgcccctaactttctttgttgcagcagctgctgcccaaccagattatgactggtgggtgcgactgctagtggcagggttagttttgtgggccggccgtaaccgtggtcaccgcatagctctgcttgtaggtccttggcctctggtagcgcttttaaccctcttgcatttggttacgcctgcttcagcttttgataccgagataattggagggctgacaataccacctgtagtagcattagttgtcatgtctcgttttggcttctttgctcacttgttacctcgctgtgctttagttaactcctatctttggcaacgttgggagaattggttttggaacgttacactaagaccggagaggtttttccttgtgctggtttgtttccccggtgcgacatatgacgcgctggtgactttctgtgtgtgtcacgtagctcttctatgtttaacatccagtgcagcatcgttctttgggactgactctagggttagggcccatagaatgttggtgcgtctcggaaagtgtcatgcttggtattctcattatgttcttaagtttttcctcttagtgtttggtgagaatggtgtgtttttctataagcacttgcatggtgatgtcttgcctaatgattttgcctcgaaactaccattgcaagagccatttttcccttttgaaggcaaggcaagggtctataggaatgaaggaagacgcttggcgtgtggggacacggttgatggtttgcccgttgttgcgcgtctcggcgaccttgttttcgcagggttggctatgccgccagatgggtgggccattaccgcaccttttacgctgcagtgtctctctgaacgtggcacgctgtcagcgatggcagtggtcatgactggtatagacccccgaacttggactggaactatcttcagattaggatctctggccactagctacatgggattcgtttgtgacaacgtgttgtatactgctcaccatggcagcaaggggcgccggttggctcatcccacaggctccatacacccaataaccgttgacgcggctaatgaccaggacatctatcaaccaccatgtggagctgggtcccttactcggtgctcttgcggggagaccaaggggtatctggtaacacgactggggtcattggttgaggtcaacaaatccgatgacccttattggtgtgtgtgcggggcccttcccatggctgttgccaagggttcttcaggtgccccgattctgtgctcctccgggcatgttattgggatgttcaccgctgctagaaattctggcggttcagtcagtcagattagggttaggccgttggtgtgtgctggataccatccccagtacacagcacatgccactcttgatacaaaaacgcgtatgcagatcttcgtgaagaccctgacgggcaagaccatcactcttgaggtcgagcccagtgacaccatcgagaatgtcaaggccaagatccaagacaaggaaggcatcccacctgaccagcagaggctgatattcgcgggcaaacagctggaggatggccgcaccctgtccgactacaacatccagaaagagtccaccttgcacctggtgctgcgtctccgcggtggcgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctactgcctttgtgggtgctggcctagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggttggggtaaacactccggcctcttaagccatttcctgttttttttttttttttttttttttttttctttttttttttctttcctttccttctttttttcctttctttttcccttctttaatggtggctccatcttagccctagtcacggctagctgtgaaaggtccgtgagccgcatgactgcagagagtgctgatactggcctctctgcagatcatgttctagactggaattcgtcgacgagctccctatagtgagtcgtattagaggccgacttggccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacaaaatattaacgtttacaatttcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacttggtcagcggccgctaatacgactcactata

SEQ ID NO: 19 - DNA, 12851 bp, Artificial Sequence
  Description of Artificial Sequence  Synthetic Primer
accacaaacactccagtttgttacactccgctaggaatgctcctggagcaccccccctagcagggcgtgggggatttcccctgcccgtctgcagaagggtggagccaaccaccttagtatgtaggcggcgggactcatgacgctcgcgtgatgacaagcgccaagcttgacttggatggccctgatgggcgttcatgggttcggtggtggtggcgctttaggcagcctccacgcccaccacctcccagatagagcggcggcactgtagggaagaccggggaccggtcactaccaaggacgcagacctctttttgagtatcacgcctccggaagtagttgggcaagcccacctatatgtgttgggatggttggggttagccatccataccgtactgcctgatagggtccttgcgaggggatctgggagtctcgtagaccgtagcacatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgtggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagcctaccaagtgcgcaattcctcggggctttaccatgtcaccaatgattgccctaactcaagtattgtgtacgaggcggccgatgccatcctgcacactccggggtgtgtcccttgcgtgcgcgagggtaacgcttcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaaatgccggccgcaccacggctgggcttgttggtctccttacaccaggcgccaagcagaacatccaactgatcaacaccaacggcagttggcacatcaatagcacggccttgaattgcaatgaaagccttaacaccggctggttagcagggctcttctatcaacacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgcttccgcaaacatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccgttgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttccttctgcttgcagacgcgcgcgtctgctcctgcttgtggatgatgttactcatatcccaagcggaggcggctttggagaacctcgtaatactcaatgcagcatccctggccgggacgcacggtcttgtgtccttcctcgtgttcttctgctttgcgtggtatctgaagggtaggtgggtgcccggagcggtctacgccctctacgggatgtggcctctcctcctgctcctgctggcgttgcctcagcgggcatacgcactggacacggaggtggccgcgtcgtgtggcggcgttgttcttgtcgggttaatggcgctgactctgtcgccatattacaagcgctatatcagctggtgcatgtggtggcttcagtattttctgaccagagtagaagcgcaactgcacgtgtgggttccccccctcaacgtccggggggggcgcgatgccgtcatcttactcatgtgtgtagtacacccgaccctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgtcttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgctggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctacgcgtatgcagatcttcgtgaagaccctgacgggcaagaccatcactcttgaggtcgagcccagtgacaccatcgagaatgtcaaggccaagatccaagacaaggaaggcatcccacctgaccagcagaggctgatattcgcgggcaaacagctggaggatggccgcaccctgtccgactacaacatccagaaagagtccaccttgcacctggtgctgcgtctccgcggtggcgcaccttttacgctgcagtgtctctctgaacgtggcacgctgtcagcgatggcagtggtcatgactggtatagacccccgaacttggactggaactatcttcagattaggatctctggccactagctacatgggattcgtttgtgacaacgtgttgtatactgctcaccatggcagcaaggggcgccggttggctcatcccacaggctccatacacccaataaccgttgacgcggctaatgaccaggacatctatcaaccaccatgtggagctgggtcccttactcggtgctcttgcggggagaccaaggggtatctggtaacacgactggggtcattggttgaggtcaacaaatccgatgacccttattggtgtgtgtgcggggcccttcccatggctgttgccaagggttcttcaggtgccccgattctgtgctcctccgggcatgttattgggatgttcaccgctgctagaaattctggcggttcagtcagtcagattagggttaggccgttggtgtgtgctggataccatccccagtacacagcacatgccactcttgatacaaaacctactgtgcctaacgagtattcagtgcaaattttaattgcccccactggcagcggcaagtcaaccaaattaccactttcttacatgcaggagaagtatgaggtcttggtcctaaatcccagtgtggctacaacagcatcaatgccaaagtacatgcacgcgacgtacggcgtgaatccaaattgctattttaatggcaaatgtaccaacacaggggcttcacttacgtacagcacatatggcatgtacctgaccggagcatgttcccggaactatgatgtaatcatttgtgacgaatgccatgctaccgatgcaaccaccgtgttgggcattggaaaggtcctaaccgaagctccatccaaaaatgttaggctagtggttcttgccacggctaccccccctggagtaatccctacaccacatgccaacataactgagattcaattaaccgatgaaggcactatcccctttcatggaaaaaagattaaggaggaaaatctgaagaaagggagacaccttatctttgaggctaccaaaaaacactgtgatgagcttgctaacgagttagctcgaaagggaataacagctgtctcttactataggggatgtgacatctcaaaaatccctgagggcgactgtgtagtagttgccactgatgccttgtgtacagggtacactggtgactttgattccgtgtatgactgcagcctcatggtagaaggcacatgccatgttgaccttgaccctactttcaccatgggtgttcgtgtgtgcggggtttcagcaatagttaaaggccagcgtaggggccgcacaggccgtgggagagctggcatatactactatgtagacgggagttgtaccccttcgggtatggttcctgaatgcaacattgttgaagccttcgacgcagccaaggcatggtatggtttgtcatcaacagaagctcaaactattctggacacctatcgcacccaacctgggttacctgcgataggagcaaatttggacgagtgggctgatctcttttctatggtcaaccccgaaccttcatttgtcaatactgcaaaaagaactgctgacaattatgttttgttgactgcagcccaactacaactgtgtcatcagtatggctatgctgctcccaatgacgcaccacggtggcagggagcccggcttgggaaaaaaccttgtggggttctgtggcgcttggacggcgctgacgcctgtcctggcccagagcccagcgaggtgaccagataccaaatgtgcttcactgaagtcaatacttctgggacagccgcactcgctgttggcgttggagtggctatggcttatctagccattgacacttttggcgccacttgtgtgcggcgttgctggtctattgcatcagtccctaccggtgctactgtcgccccagtggttgacgaagaagaaatcgtggaggagtgtgcatcattcattcccttggaggccatggttgctgcaatcgataagctgaagagtacaataaccacaactagtcctttcacattggaaaccgcccttgaaaaacttaacacctttcttgggcctcatgcagctacaatccttgctatcatagagtattgctgtggtttagtcactttacctgacaatccctttgcatcatgcgtgtttgctttcattgcgggtattactaccccactacctcacaagatcaaaatgttcctgtcattatttggaggcgcaattgcgtccaagcttacagacgctagaggcgcactggcgttcatgatggccggggctgcgggaacagctcttggtacatggacatcggtgggttttgtctttgacatgctaggcggctatgctgccgcctcatccactgcttgcttgacatttaaatgcttgatgggtgagtggcccactatggatcagcttgctggtttagtctactccgcgttcaatccggccgcaggagttgtgggcgtcttgtcagcttgtgcaatgtttgctttgacaacagcagggccagatcactggcccaacagacttcttactatgcttgctaggagcaacactgtatgtaatgagtactttattgccactcgtgacatccgcaggaagatactgggcattctggaggcatctaccccctggagtgtcatatcagcttgcatccgttggctccacaccccgacggaggatgattgcggcctcattgcttggggtctagagatttggcagtatgtgtgcaatttctttgtgatttgctttaatgtccttaaagctggagttcagagcatggttaacattcctggttgtcctttctacagctgccagaaggggtacaagggcccctggattggatcaggtatgctccaagcacgctgtccatgcggtgctgaactcatcttttctgttgagaatggttttgcaaaactttacaaaggacccagaacttgttcaaattactggagaggggctgttccagtcaacgctaggctgtgtgggtcggctagaccggacccaactgattggactagtcttgtcgtcaattatggcgttagggactactgtaaatatgagaaaatgggagatcacatttttgttacagcagtatcctctccaaatgtctgtttcacccaggtgcccccaaccttgagagctgcagtggccgtggacggcgtacaggttcagtgttatctaggtgagcccaaaactccttggacgacatctgcttgctgttacggtcctgacggtaagggtaaaactgttaagcttcccttccgcgttgacggtcacacacctggtgtgcgcatgcaacttaatttgcgtgatgcacttgagacaaatgactgtaattccacaaacaacactcctagtgatgaagccgcagtgtccgctcttgttttcaaacaggagttgcggcgtacaaaccaattgcttgaggcaatttcagctggcgttgacaccaccaaactgccagccccctccatcgaagaggtagtggtaagaaagcgccagttccgggcaagaactggttcgcttaccttgcctccccctccgagatccgtcccaggagtgtcatgtcctgaaagcctgcaacgaagtgacccgttagaaggtccttcaaacctccctccttcaccacctgttctacagttggccatgccgatgcccctgttgggagcgggtgagtgtaaccctttcactgcaattggatgtgcaatgaccgaaacaggcggaggccctgatgatttacccagttaccctcccaaaaaggaggtctctgaatggtcagacgaaagttggtcaacggctacaaccgtttccagctacgttactggccccccgtaccctaagatacggggaaaggattccactcagtcagcccccgccaaacggcctacaaaaaagaagttgggaaagagtgagttttcgtgcagcatgagctacacctggaccgacgtgattagcttcaaaactgcttctaaagttctgtctgcaactcgggccatcactagtggtttcctcaaacaaagatcattggtgtatgtgactgagccgcgggatgcggagcttagaaaacaaaaagtcactattaatagacaacctctgttccccccatcataccacaagcaagtgagattggctaaggaaaaagcttcaaaagttgtcggtgtcatgtgggactatgatgaagtagcagctcacacgccctctaagtctgctaagtcccacatcactggccttcggggcactgatgttcgttctggagcagcccgcaaggctgttctggacttgcagaagtgtgtcgaggcaggtgagataccgagtcattatcggcaaactgtgatagttccaaaggaggaggtcttcgtgaagaccccccagaaaccaacaaagaaacccccaaggcttatctcgtacccccaccttgaaatgagatgtgttgagaagatgtactacggtcaggttgctcctgacgtagttaaagctgtcatgggagatgcgtacgggtttgtcgacccacgtacccgtgtcaagcgtctgttgtcgatgtggtcacccgatgcagtcggagccacatgcgatacagtgtgttttgacagtaccatcacacccgaggatatcatggtggagacagacatctactcagcagctaaactcagtgaccaacaccgagctggcattcacaccattgcgaggcagttatacgctggaggaccgatgatcgcttatgatggccgagagatcggatatcgtaggtgtaggtcttccggcgtctatactacctcaagttccaacagtttgacctgctggctgaaggtaaatgctgcagccgaacaggctggcatgaagaaccctcgcttccttatttgcggcgatgattgcaccgtaatttggaagagcgccggagcagatgcagacaaacaagcaatgcgtgtctttgctagctggatgaaggtgatgggtgcaccacaagattgtgtgcctcaacccaaatacagtttggaagaattaacatcatgctcatcaaatgttacctctggaattaccaaaagtggcaagccttactactttcttacaagagatcctcgtatcccccttggcaggtgctctgccgagggtctgggatacaaccccagtgctgcgtggattgggtatctaatacatcactacccatgtttgtgggttagccgtgtgttggctgtccatttcatggagcagatgctctttgaggacaaacttcccgagacggtgacctttgactggtatgggaaaaattatacggtgcctgtagaagatctgcccagcatcattgctggtgtgcacggtattgaggctttctcggtggtgcgctacaccaacgctgagatcctcagagtttcccaatcactaacagacatgaccatgccccccctgcgagcctggcgaaagaaagccagggcggtcctcgccagcgccaagaggcgtggcggagcacacgcaaaattggctcgcttccttctctggcatgctacatctagacctctaccagatttggataagacgagcgtggctcggtacaccactttcaattattgtgatgtttactccccggagggggatgtgtttattacaccacagagaagattgcagaagtttcttgtgaagtatttggctgtcattgtttttgccctagggctcattgctgttggattagccatcagctgaacccccaaattcaaaattaactaacagtttttttttttttttttttttttagggcagcggcaacaggggagaccccgggcttaacgaccccgccgatgtgagtttggcgaccatggtggatcagaaccgtttcgggtgaagccatggtctgaaggggatgacgtcccttctggctcatccacaaaaaccgtctcgggtgggtgaggagtcctggctgtgtgggaagcagtcagtataattcccgtcgtgtgtggtgacgcctcacgacgtatttgtccgctgtgcagagcgtagtaccaagggctgcaccccggtttttgttccaagcggagggcaacccccgcttggaattaaaaactcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacctgaggtaattataacccgggccctatatatggatcccgtaatacgactcactatag

SEQ ID NO: 20 - DNA, 12023 bp, Artificial Sequence
  Description of Artificial Sequence  Synthetic Primer
accacaaacactccagtttgttacactccgctaggaatgctcctggagcaccccccctagcagggcgtgggggatttcccctgcccgtctgcagaagggtggagccaaccaccttagtatgtaggcggcgggactcatgacgctcgcgtgatgacaagcgccaagcttgacttggatggccctgatgggcgttcatgggttcggtggtggtggcgctttaggcagcctccacgcccaccacctcccagatagagcggcggcactgtagggaagaccggggaccggtcactaccaaggacgcagacctctttttgagtatcacgcctccggaagtagttgggcaagcccacctatatgtgttgggatggttggggttagccatccataccgtactgcctgatagggtccttgcgaggggatctgggagtctcgtagaccgtagcacatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgtggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagcctaccaagtgcgcaattcctcggggctttaccatgtcaccaatgattgccctaactcaagtattgtgtacgaggcggccgatgccatcctgcacactccggggtgtgtcccttgcgtgcgcgagggtaacgcttcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaaatgccggccgcaccacggctgggcttgttggtctccttacaccaggcgccaagcagaacatccaactgatcaacaccaacggcagttggcacatcaatagcacggccttgaattgcaatgaaagccttaacaccggctggttagcagggctcttctatcaacacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgcttccgcaaacatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccgttgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttccttctgcttgcagacgcgcgcgtctgctcctgcttgtggatgatgttactcatatcccaagcggaggcggctttggagaacctcgtaatactcaatgcagcatccctggccgggacgcacggtcttgtgtccttcctcgtgttcttctgctttgcgtggtatctgaagggtaggtgggtgcccggagcggtctacgccctctacgggatgtggcctctcctcctgctcctgctggcgttgcctcagcgggcatacgcatttgataccgagataattggagggctgacaataccacctgtagtagcattagttgtcatgtctcgttttggcttctttgctcacttgttacctcgctgtgctttagttaactcctatctttggcaacgttgggagaattggttttggaacgttacactaagaccggagaggtttttccttgtgctggtttgtttccccggtgcgacatatgacgcgctggtgactttctgtgtgtgtcacgtagctcttctatgtttaacatccagtgcagcatcgttctttgggactgactctagggttagggcccatagaatgttggtgcgtctcggaaagtgtcatgcttggtattctcattatgttcttaagtttttcctcttagtgtttggtgagaatggtgtgtttttctataagcacttgcatggtgatgtcttgcctaatgattttgcctcgaaactaccattgcaagagccatttttcccttttgaaggcaaggcaagggtctataggaatgaaggaagacgcttggcgtgtggggacacggttgatggtttgcccgttgttgcgcgtctcggcgaccttgttttcgcagggttggctatgccgccagatgggtgggccattaccgcaccttttacgctgcagtgtctctctgaacgtggcacgctgtcagcgatggcagtggtcatgactggtatagacccccgaacttggactggaactatcttcagattaggatctctggccactagctacatgggattcgtttgtgacaacgtgttgtatactgctcaccatggcagcaaggggcgccggttggctcatcccacaggctccatacacccaataaccgttgacgcggctaatgaccaggacatctatcaaccaccatgtggagctgggtcccttactcggtgctcttgcggggagaccaaggggtatctggtaacacgactggggtcattggttgaggtcaacaaatccgatgacccttattggtgtgtgtgcggggcccttcccatggctgttgccaagggttcttcaggtgccccgattctgtgctcctccgggcatgttattgggatgttcaccgctgctagaaattctggcggttcagtcagtcagattagggttaggccgttggtgtgtgctggataccatccccagtacacagcacatgccactcttgatacaaaacctactgtgcctaacgagtattcagtgcaaattttaattgcccccactggcagcggcaagtcaaccaaattaccactttcttacatgcaggagaagtatgaggtcttggtcctaaatcccagtgtggctacaacagcatcaatgccaaagtacatgcacgcgacgtacggcgtgaatccaaattgctattttaatggcaaatgtaccaacacaggggcttcacttacgtacagcacatatggcatgtacctgaccggagcatgttcccggaactatgatgtaatcatttgtgacgaatgccatgctaccgatgcaaccaccgtgttgggcattggaaaggtcctaaccgaagctccatccaaaaatgttaggctagtggttcttgccacggctaccccccctggagtaatccctacaccacatgccaacataactgagattcaattaaccgatgaaggcactatcccctttcatggaaaaaagattaaggaggaaaatctgaagaaagggagacaccttatctttgaggctaccaaaaaacactgtgatgagcttgctaacgagttagctcgaaagggaataacagctgtctcttactataggggatgtgacatctcaaaaatccctgagggcgactgtgtagtagttgccactgatgccttgtgtacagggtacactggtgactttgattccgtgtatgactgcagcctcatggtagaaggcacatgccatgttgaccttgaccctactttcaccatgggtgttcgtgtgtgcggggtttcagcaatagttaaaggccagcgtaggggccgcacaggccgtgggagagctggcatatactactatgtagacgggagttgtaccccttcgggtatggttcctgaatgcaacattgttgaagccttcgacgcagccaaggcatggtatggtttgtcatcaacagaagctcaaactattctggacacctatcgcacccaacctgggttacctgcgataggagcaaatttggacgagtgggctgatctcttttctatggtcaaccccgaaccttcatttgtcaatactgcaaaaagaactgctgacaattatgttttgttgactgcagcccaactacaactgtgtcatcagtatggctatgctgctcccaatgacgcaccacggtggcagggagcccggcttgggaaaaaaccttgtggggttctgtggcgcttggacggcgctgacgcctgtcctggcccagagcccagcgaggtgaccagataccaaatgtgcttcactgaagtcaatacttctgggacagccgcactcgctgttggcgttggagtggctatggcttatctagccattgacacttttggcgccacttgtgtgcggcgttgctggtctattgcatcagtccctaccggtgctactgtcgccccagtggttgacgaagaagaaatcgtggaggagtgtgcatcattcattcccttggaggccatggttgctgcaatcgataagctgaagagtacaatcaccacaactagtcctttcacattggaaaccgcccttgaaaaacttaacacctttcttgggcctcatgcagctacaatccttgctatcatagagtattgctgtggtttagtcactttacctgacaatccctttgcatcatgcgtgtttgctttcattgcgggtattactaccccactacctcacaagatcaaaatgttcctgtcattatttggaggcgcaattgcgtccaagcttacagacgctagaggcgcactggcgttcatgatggccggggctgcgggaacagctcttggtacatggacatcggtgggttttgtctttgacatgctaggcggctatgctgccgcctcatccactgcttgcttgacatttaaatgcttgatgggtgagtggcccactatggatcagcttgctggtttagtctactccgcgttcaatccggccgcaggagttgtgggcgtcttgtcagcttgtgcaatgtttgctttgacaacagcagggccagatcactggcccaacagacttcttactatgcttgctaggagcaacactgtatgtaatgagtactttattgccactcgtgacatccgcaggaagatactgggcattctggaggcatctaccccctggagtgtcatatcagcttgcatccgttggctccacaccccgacggaggatgattgcggcctcattgcttggggtctagagatttggcagtatgtgtgcaatttctttgtgatttgctttaatgtccttaaagctggagttcagagcatggttaacattcctggttgtcctttctacagctgccagaaggggtacaagggcccctggattggatcaggtatgctccaagcacgctgtccatgcggtgctgaactcatcttttctgttgagaatggttttgcaaaactttacaaaggacccagaacttgttcaaattactggagaggggctgttccagtcaacgctaggctgtgtgggtcggctagaccggacccaactgattggactagtcttgtcgtcaattatggcgttagggactactgtaaatatgagaaaatgggagatcacatttttgttacagcagtatcctctccaaatgtctgtttcacccaggtgcccccaaccttgagagctgcagtggccgtggacggcgtacaggttcagtgttatctaggtgagcccaaaactccttggacgacatctgcttgctgttacggtcctgacggtaagggtaaaactgttaagcttcccttccgcgttgacggtcacacacctggtgtgcgcatgcaacttaatttgcgtgatgcacttgagacaaatgactgtaattccacaaacaacactcctagtgatgaagccgcagtgtccgctcttgttttcaaacaggagttgcggcgtacaaaccaattgcttgaggcaatttcagctggcgttgacaccaccaaactgccagccccctccatcgaagaggtagtggtaagaaagcgccagttccgggcaagaactggttcgcttaccttgcctccccctccgagatccgtcccaggagtgtcatgtcctgaaagcctgcaacgaagtgacccgttagaaggtccttcaaacctccctccttcaccacctgttctacagttggccatgccgatgcccctgttgggagcgggtgagtgtaaccctttcactgcaattggatgtgcaatgaccgaaacaggcggaggccctgatgatttacccagttaccctcccaaaaaggaggtctctgaatggtcagacgaaagttggtcaacggctacaaccgtttccagctacgttactggccccccgtaccctaagatacggggaaaggattccactcagtcagcccccgccaaacggcctacaaaaaagaagttgggaaagagtgagttttcgtgcagcatgagctacacctggaccgacgtgattagcttcaaaactgcttctaaagttctgtctgcaactcgggccatcactagtggtttcctcaaacaaagatcattggtgtatgtgactgagccgcgggatgcggagcttagaaaacaaaaagtcactattaatagacaacctctgttccccccatcataccacaagcaagtgagattggctaaggaaaaagcttcaaaagttgtcggtgtcatgtgggactatgatgaagtagcagctcacacgccctctaagtctgctaagtcccacatcactggccttcggggcactgatgttcgttctggagcagcccgcaaggctgttctggacttgcagaagtgtgtcgaggcaggtgagataccgagtcattatcggcaaactgtgatagttccaaaggaggaggtcttcgtgaagaccccccagaaaccaacaaagaaacccccaaggcttatctcgtacccccaccttgaaatgagatgtgttgagaagatgtactacggtcaggttgctcctgacgtagttaaagctgtcatgggagatgcgtacgggtttgtcgacccacgtacccgtgtcaagcgtctgttgtcgatgtggtcacccgatgcagtcggagccacatgcgatacagtgtgttttgacagtaccatcacacccgaggatatcatggtggagacagacatctactcagcagctaaactcagtgaccaacaccgagctggcattcacaccattgcgaggcagttatacgctggaggaccgatgatcgcttatgatggccgagagatcggatatcgtaggtgtaggtcttccggcgtctatactacctcaagttccaacagtttgacctgctggctgaaggtaaatgctgcagccgaacaggctggcatgaagaaccctcgcttccttatttgcggcgatgattgcaccgtaatttggaagagcgccggagcagatgcagacaaacaagcaatgcgtgtctttgctagctggatgaaggtgatgggtgcaccacaagattgtgtgcctcaacccaaatacagtttggaagaattaacatcatgctcatcaaatgttacctctggaattaccaaaagtggcaagccttactactttcttacaagagatcctcgtatcccccttggcaggtgctctgccgagggtctgggatacaaccccagtgctgcgtggattgggtatctaatacatcactacccatgtttgtgggttagccgtgtgttggctgtccatttcatggagcagatgctctttgaggacaaacttcccgagacggtgacctttgactggtatgggaaaaattatacggtgcctgtagaagatctgcccagcatcattgctggtgtgcacggtattgaggctttctcggtggtgcgctacaccaacgctgagatcctcagagtttcccaatcactaacagacatgaccatgccccccctgcgagcctggcgaaagaaagccagggcggtcctcgccagcgccaagaggcgtggcggagcacacgcaaaattggctcgcttccttctctggcatgctacatctagacctctaccagatttggataagacgagcgtggctcggtacaccactttcaattattgtgatgtttactccccggagggggatgtgtttattacaccacagagaagattgcagaagtttcttgtgaagtatttggctgtcattgtttttgccctagggctcattgctgttggattagccatcagctgaacccccaaattcaaaattaactaacagtttttttttttttttttttttttagggcagcggcaacaggggagaccccgggcttaacgaccccgccgatgtgagtttggcgaccatggtggatcagaaccgtttcgggtgaagccatggtctgaaggggatgacgtcccttctggctcatccacaaaaaccgtctcgggtgggtgaggagtcctggctgtgtgggaagcagtcagtataattcccgtcgtgtgtggtgacgcctcacgacgtatttgtccgctgtgcagagcgtagtaccaagggctgcaccccggtttttgttccaagcggagggcaacccccgcttggaattaaaaactcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacctgaggtaattataacccgggccctatatatggatcccgtaatacgactcactatag

SEQ ID NO: 21 - DNA, 12746 bp, Artificial Sequence
  Description of Artificial Sequence  Synthetic Primer
accacaaacactccagtttgttacactccgctaggaatgctcctggagcaccccccctagcagggcgtgggggatttcccctgcccgtctgcagaagggtggagccaaccaccttagtatgtaggcggcgggactcatgacgctcgcgtgatgacaagcgccaagcttgacttggatggccctgatgggcgttcatgggttcggtggtggtggcgctttaggcagcctccacgcccaccacctcccagatagagcggcggcactgtagggaagaccggggaccggtcactaccaaggacgcagacctctttttgagtatcacgcctccggaagtagttgggcaagcccacctatatgtgttgggatggttggggttagccatccataccgtactgcctgatagggtccttgcgaggggatctgggagtctcgtagaccgtagcacatgcctgttatttctactcaaacaagtcctgtacctgcgcccagaacgcgcaagaacaagcagacgcaggcttcatatcctgtgtccattaaaacatctgttgaaaggggacaacgagcaaagcgcaaagtccagcgcgatgctcggcctcgtaattacaaaattgctggtatccatgatggcttgcagacattggctcaggctgctttgccagctcatggttggggacgccaagaccctcgccataagtctcgcaatcttggaatccttctggattaccctttggggtggattggtgatgttacaactcacacacctctagtaggcccgctggtggcaggagcggtcgttcgaccagtctgccagatagtacgcttgctggaggatggagtcaactgggctactggttggttcggtgtccacctttttgtggtatgtctgctatctttggcctgtccctgtagtgggtaccaagtgcgcaattcctcggggctttaccatgtcaccaatgattgccctaactcaagtattgtgtacgaggcggccgatgccatcctgcacactccggggtgtgtcccttgcgtgcgcgagggtaacgcttcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaaatgccggccgcaccacggctgggcttgttggtctccttacaccaggcgccaagcagaacatccaactgatcaacaccaacggcagttggcacatcaatagcacggccttgaattgcaatgaaagccttaacaccggctggttagcagggctcttctatcaacacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgcttccgcaaacatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccgttgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttccttctgcttgcagacgcgcgcgtctgctcctgcttgtggatgatgttactcatatcccaagcggaggcggctttggagaacctcgtaatactcaatgcagcatccctggccgggacgcacggtcttgtgtccttcctcgtgttcttctgctttgcgtggtatctgaagggtaggtgggtgcccggagcggtctacgccctctacgggatgtggcctctcctcctgctcctgctggcgttgcctcagcgggcatacgcactggacacggaggtggccgcgtcgtgtggcggcgttgttcttgtcgggttaatggcgctgactctgtcgccatattacaagcgctatatcagctggtgcatgtggtggcttcagtattttctgaccagagtagaagcgcaactgcacgtgtgggttccccccctcaacgtccggggggggcgcgatgccgtcatcttactcatgtgtgtagtacacccgaccctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgtcttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgctggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctacgcgtatgcagatcttcgtgaagaccctgacgggcaagaccatcactcttgaggtcgagcccagtgacaccatcgagaatgtcaaggccaagatccaagacaaggaaggcatcccacctgaccagcagaggctgatattcgcgggcaaacagctggaggatggccgcaccctgtccgactacaacatccagaaagagtccaccttgcacctggtgctgcgtctccgcggtggcgcaccttttacgctgcagtgtctctctgaacgtggcacgctgtcagcgatggcagtggtcatgactggtatagacccccgaacttggactggaactatcttcagattaggatctctggccactagctacatgggattcgtttgtgacaacgtgttgtatactgctcaccatggcagcaaggggcgccggttggctcatcccacaggctccatacacccaataaccgttgacgcggctaatgaccaggacatctatcaaccaccatgtggagctgggtcccttactcggtgctcttgcggggagaccaaggggtatctggtaacacgactggggtcattggttgaggtcaacaaatccgatgacccttattggtgtgtgtgcggggcccttcccatggctgttgccaagggttcttcaggtgccccgattctgtgctcctccgggcatgttattgggatgttcaccgctgctagaaattctggcggttcagtcagtcagattagggttaggccgttggtgtgtgctggataccatccccagtacacagcacatgccactcttgatacaaaacctactgtgcctaacgagtattcagtgcaaattttaattgcccccactggcagcggcaagtcaaccaaattaccactttcttacatgcaggagaagtatgaggtcttggtcctaaatcccagtgtggctacaacagcatcaatgccaaagtacatgcacgcgacgtacggcgtgaatccaaattgctattttaatggcaaatgtaccaacacaggggcttcacttacgtacagcacatatggcatgtacctgaccggagcatgttcccggaactatgatgtaatcatttgtgacgaatgccatgctaccgatgcaaccaccgtgttgggcattggaaaggtcctaaccgaagctccatccaaaaatgttaggctagtggttcttgccacggctaccccccctggagtaatccctacaccacatgccaacataactgagattcaattaaccgatgaaggcactatcccctttcatggaaaaaagattaaggaggaaaatctgaagaaagggagacaccttatctttgaggctaccaaaaaacactgtgatgagcttgctaacgagttagctcgaaagggaataacagctgtctcttactataggggatgtgacatctcaaaaatccctgagggcgactgtgtagtagttgccactgatgccttgtgtacagggtacactggtgactttgattccgtgtatgactgcagcctcatggtagaaggcacatgccatgttgaccttgaccctactttcaccatgggtgttcgtgtgtgcggggtttcagcaatagttaaaggccagcgtaggggccgcacaggccgtgggagagctggcatatactactatgtagacgggagttgtaccccttcgggtatggttcctgaatgcaacattgttgaagccttcgacgcagccaaggcatggtatggtttgtcatcaacagaagctcaaactattctggacacctatcgcacccaacctgggttacctgcgataggagcaaatttggacgagtgggctgatctcttttctatggtcaaccccgaaccttcatttgtcaatactgcaaaaagaactgctgacaattatgttttgttgactgcagcccaactacaactgtgtcatcagtatggctatgctgctcccaatgacgcaccacggtggcagggagcccggcttgggaaaaaaccttgtggggttctgtggcgcttggacggcgctgacgcctgtcctggcccagagcccagcgaggtgaccagataccaaatgtgcttcactgaagtcaatacttctgggacagccgcactcgctgttggcgttggagtggctatggcttatctagccattgacacttttggcgccacttgtgtgcggcgttgctggtctattgcatcagtccctaccggtgctactgtcgccccagtggttgacgaagaagaaatcgtggaggagtgtgcatcattcattcccttggaggccatggttgctgcaatcgataagctgaagagtacaataaccacaactagtcctttcacattggaaaccgcccttgaaaaacttaacacctttcttgggcctcatgcagctacaatccttgctatcatagagtattgctgtggtttagtcactttacctgacaatccctttgcatcatgcgtgtttgctttcattgcgggtattactaccccactacctcacaagatcaaaatgttcctgtcattatttggaggcgcaattgcgtccaagcttacagacgctagaggcgcactggcgttcatgatggccggggctgcgggaacagctcttggtacatggacatcggtgggttttgtctttgacatgctaggcggctatgctgccgcctcatccactgcttgcttgacatttaaatgcttgatgggtgagtggcccactatggatcagcttgctggtttagtctactccgcgttcaatccggccgcaggagttgtgggcgtcttgtcagcttgtgcaatgtttgctttgacaacagcagggccagatcactggcccaacagacttcttactatgcttgctaggagcaacactgtatgtaatgagtactttattgccactcgtgacatccgcaggaagatactgggcattctggaggcatctaccccctggagtgtcatatcagcttgcatccgttggctccacaccccgacggaggatgattgcggcctcattgcttggggtctagagatttggcagtatgtgtgcaatttctttgtgatttgctttaatgtccttaaagctggagttcagagcatggttaacattcctggttgtcctttctacagctgccagaaggggtacaagggcccctggattggatcaggtatgctccaagcacgctgtccatgcggtgctgaactcatcttttctgttgagaatggttttgcaaaactttacaaaggacccagaacttgttcaaattactggagaggggctgttccagtcaacgctaggctgtgtgggtcggctagaccggacccaactgattggactagtcttgtcgtcaattatggcgttagggactactgtaaatatgagaaaatgggagatcacatttttgttacagcagtatcctctccaaatgtctgtttcacccaggtgcccccaaccttgagagctgcagtggccgtggacggcgtacaggttcagtgttatctaggtgagcccaaaactccttggacgacatctgcttgctgttacggtcctgacggtaagggtaaaactgttaagcttcccttccgcgttgacggtcacacacctggtgtgcgcatgcaacttaatttgcgtgatgcacttgagacaaatgactgtaattccacaaacaacactcctagtgatgaagccgcagtgtccgctcttgttttcaaacaggagttgcggcgtacaaaccaattgcttgaggcaatttcagctggcgttgacaccaccaaactgccagccccctccatcgaagaggtagtggtaagaaagcgccagttccgggcaagaactggttcgcttaccttgcctccccctccgagatccgtcccaggagtgtcatgtcctgaaagcctgcaacgaagtgacccgttagaaggtccttcaaacctccctccttcaccacctgttctacagttggccatgccgatgcccctgttgggagcgggtgagtgtaaccctttcactgcaattggatgtgcaatgaccgaaacaggcggaggccctgatgatttacccagttaccctcccaaaaaggaggtctctgaatggtcagacgaaagttggtcaacggctacaaccgtttccagctacgttactggccccccgtaccctaagatacggggaaaggattccactcagtcagcccccgccaaacggcctacaaaaaagaagttgggaaagagtgagttttcgtgcagcatgagctacacctggaccgacgtgattagcttcaaaactgcttctaaagttctgtctgcaactcgggccatcactagtggtttcctcaaacaaagatcattggtgtatgtgactgagccgcgggatgcggagcttagaaaacaaaaagtcactattaatagacaacctctgttccccccatcataccacaagcaagtgagattggctaaggaaaaagcttcaaaagttgtcggtgtcatgtgggactatgatgaagtagcagctcacacgccctctaagtctgctaagtcccacatcactggccttcggggcactgatgttcgttctggagcagcccgcaaggctgttctggacttgcagaagtgtgtcgaggcaggtgagataccgagtcattatcggcaaactgtgatagttccaaaggaggaggtcttcgtgaagaccccccagaaaccaacaaagaaacccccaaggcttatctcgtacccccaccttgaaatgagatgtgttgagaagatgtactacggtcaggttgctcctgacgtagttaaagctgtcatgggagatgcgtacgggtttgtcgacccacgtacccgtgtcaagcgtctgttgtcgatgtggtcacccgatgcagtcggagccacatgcgatacagtgtgttttgacagtaccatcacacccgaggatatcatggtggagacagacatctactcagcagctaaactcagtgaccaacaccgagctggcattcacaccattgcgaggcagttatacgctggaggaccgatgatcgcttatgatggccgagagatcggatatcgtaggtgtaggtcttccggcgtctatactacctcaagttccaacagtttgacctgctggctgaaggtaaatgctgcagccgaacaggctggcatgaagaaccctcgcttccttatttgcggcgatgattgcaccgtaatttggaagagcgccggagcagatgcagacaaacaagcaatgcgtgtctttgctagctggatgaaggtgatgggtgcaccacaagattgtgtgcctcaacccaaatacagtttggaagaattaacatcatgctcatcaaatgttacctctggaattaccaaaagtggcaagccttactactttcttacaagagatcctcgtatcccccttggcaggtgctctgccgagggtctgggatacaaccccagtgctgcgtggattgggtatctaatacatcactacccatgtttgtgggttagccgtgtgttggctgtccatttcatggagcagatgctctttgaggacaaacttcccgagacggtgacctttgactggtatgggaaaaattatacggtgcctgtagaagatctgcccagcatcattgctggtgtgcacggtattgaggctttctcggtggtgcgctacaccaacgctgagatcctcagagtttcccaatcactaacagacatgaccatgccccccctgcgagcctggcgaaagaaagccagggcggtcctcgccagcgccaagaggcgtggcggagcacacgcaaaattggctcgcttccttctctggcatgctacatctagacctctaccagatttggataagacgagcgtggctcggtacaccactttcaattattgtgatgtttactccccggagggggatgtgtttattacaccacagagaagattgcagaagtttcttgtgaagtatttggctgtcattgtttttgccctagggctcattgctgttggattagccatcagctgaacccccaaattcaaaattaactaacagtttttttttttttttttttttttagggcagcggcaacaggggagaccccgggcttaacgaccccgccgatgtgagtttggcgaccatggtggatcagaaccgtttcgggtgaagccatggtctgaaggggatgacgtcccttctggctcatccacaaaaaccgtctcgggtgggtgaggagtcctggctgtgtgggaagcagtcagtataattcccgtcgtgtgtggtgacgcctcacgacgtatttgtccgctgtgcagagcgtagtaccaagggctgcaccccggtttttgttccaagcggagggcaacccccgcttggaattaaaaactcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacctgaggtaattataacccgggccctatatatggatcccgtaatacgactcactatag

SEQ ID NO: 22 - DNA, 11918 bp, Artificial Sequence
  Description of Artificial Sequence  Synthetic Primer
accacaaacactccagtttgttacactccgctaggaatgctcctggagcaccccccctagcagggcgtgggggatttcccctgcccgtctgcagaagggtggagccaaccaccttagtatgtaggcggcgggactcatgacgctcgcgtgatgacaagcgccaagcttgacttggatggccctgatgggcgttcatgggttcggtggtggtggcgctttaggcagcctccacgcccaccacctcccagatagagcggcggcactgtagggaagaccggggaccggtcactaccaaggacgcagacctctttttgagtatcacgcctccggaagtagttgggcaagcccacctatatgtgttgggatggttggggttagccatccataccgtactgcctgatagggtccttgcgaggggatctgggagtctcgtagaccgtagcacatgcctgttatttctactcaaacaagtcctgtacctgcgcccagaacgcgcaagaacaagcagacgcaggcttcatatcctgtgtccattaaaacatctgttgaaaggggacaacgagcaaagcgcaaagtccagcgcgatgctcggcctcgtaattacaaaattgctggtatccatgatggcttgcagacattggctcaggctgctttgccagctcatggttggggacgccaagaccctcgccataagtctcgcaatcttggaatccttctggattaccctttggggtggattggtgatgttacaactcacacacctctagtaggcccgctggtggcaggagcggtcgttcgaccagtctgccagatagtacgcttgctggaggatggagtcaactgggctactggttggttcggtgtccacctttttgtggtatgtctgctatctttggcctgtccctgtagtgggtaccaagtgcgcaattcctcggggctttaccatgtcaccaatgattgccctaactcaagtattgtgtacgaggcggccgatgccatcctgcacactccggggtgtgtcccttgcgtgcgcgagggtaacgcttcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaaatgccggccgcaccacggctgggcttgttggtctccttacaccaggcgccaagcagaacatccaactgatcaacaccaacggcagttggcacatcaatagcacggccttgaattgcaatgaaagccttaacaccggctggttagcagggctcttctatcaacacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgcttccgcaaacatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccgttgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttccttctgcttgcagacgcgcgcgtctgctcctgcttgtggatgatgttactcatatcccaagcggaggcggctttggagaacctcgtaatactcaatgcagcatccctggccgggacgcacggtcttgtgtccttcctcgtgttcttctgctttgcgtggtatctgaagggtaggtgggtgcccggagcggtctacgccctctacgggatgtggcctctcctcctgctcctgctggcgttgcctcagcgggcatacgcatttgataccgagataattggagggctgacaataccacctgtagtagcattagttgtcatgtctcgttttggcttctttgctcacttgttacctcgctgtgctttagttaactcctatctttggcaacgttgggagaattggttttggaacgttacactaagaccggagaggtttttccttgtgctggtttgtttccccggtgcgacatatgacgcgctggtgactttctgtgtgtgtcacgtagctcttctatgtttaacatccagtgcagcatcgttctttgggactgactctagggttagggcccatagaatgttggtgcgtctcggaaagtgtcatgcttggtattctcattatgttcttaagtttttcctcttagtgtttggtgagaatggtgtgtttttctataagcacttgcatggtgatgtcttgcctaatgattttgcctcgaaactaccattgcaagagccatttttcccttttgaaggcaaggcaagggtctataggaatgaaggaagacgcttggcgtgtggggacacggttgatggtttgcccgttgttgcgcgtctcggcgaccttgttttcgcagggttggctatgccgccagatgggtgggccattaccgcaccttttacgctgcagtgtctctctgaacgtggcacgctgtcagcgatggcagtggtcatgactggtatagacccccgaacttggactggaactatcttcagattaggatctctggccactagctacatgggattcgtttgtgacaacgtgttgtatactgctcaccatggcagcaaggggcgccggttggctcatcccacaggctccatacacccaataaccgttgacgcggctaatgaccaggacatctatcaaccaccatgtggagctgggtcccttactcggtgctcttgcggggagaccaaggggtatctggtaacacgactggggtcattggttgaggtcaacaaatccgatgacccttattggtgtgtgtgcggggcccttcccatggctgttgccaagggttcttcaggtgccccgattctgtgctcctccgggcatgttattgggatgttcaccgctgctagaaattctggcggttcagtcagtcagattagggttaggccgttggtgtgtgctggataccatccccagtacacagcacatgccactcttgatacaaaacctactgtgcctaacgagtattcagtgcaaattttaattgcccccactggcagcggcaagtcaaccaaattaccactttcttacatgcaggagaagtatgaggtcttggtcctaaatcccagtgtggctacaacagcatcaatgccaaagtacatgcacgcgacgtacggcgtgaatccaaattgctattttaatggcaaatgtaccaacacaggggcttcacttacgtacagcacatatggcatgtacctgaccggagcatgttcccggaactatgatgtaatcatttgtgacgaatgccatgctaccgatgcaaccaccgtgttgggcattggaaaggtcctaaccgaagctccatccaaaaatgttaggctagtggttcttgccacggctaccccccctggagtaatccctacaccacatgccaacataactgagattcaattaaccgatgaaggcactatcccctttcatggaaaaaagattaaggaggaaaatctgaagaaagggagacaccttatctttgaggctaccaaaaaacactgtgatgagcttgctaacgagttagctcgaaagggaataacagctgtctcttactataggggatgtgacatctcaaaaatccctgagggcgactgtgtagtagttgccactgatgccttgtgtacagggtacactggtgactttgattccgtgtatgactgcagcctcatggtagaaggcacatgccatgttgaccttgaccctactttcaccatgggtgttcgtgtgtgcggggtttcagcaatagttaaaggccagcgtaggggccgcacaggccgtgggagagctggcatatactactatgtagacgggagttgtaccccttcgggtatggttcctgaatgcaacattgttgaagccttcgacgcagccaaggcatggtatggtttgtcatcaacagaagctcaaactattctggacacctatcgcacccaacctgggttacctgcgataggagcaaatttggacgagtgggctgatctcttttctatggtcaaccccgaaccttcatttgtcaatactgcaaaaagaactgctgacaattatgttttgttgactgcagcccaactacaactgtgtcatcagtatggctatgctgctcccaatgacgcaccacggtggcagggagcccggcttgggaaaaaaccttgtggggttctgtggcgcttggacggcgctgacgcctgtcctggcccagagcccagcgaggtgaccagataccaaatgtgcttcactgaagtcaatacttctgggacagccgcactcgctgttggcgttggagtggctatggcttatctagccattgacacttttggcgccacttgtgtgcggcgttgctggtctattgcatcagtccctaccggtgctactgtcgccccagtggttgacgaagaagaaatcgtggaggagtgtgcatcattcattcccttggaggccatggttgctgcaatcgataagctgaagagtacaatcaccacaactagtcctttcacattggaaaccgcccttgaaaaacttaacacctttcttgggcctcatgcagctacaatccttgctatcatagagtattgctgtggtttagtcactttacctgacaatccctttgcatcatgcgtgtttgctttcattgcgggtattactaccccactacctcacaagatcaaaatgttcctgtcattatttggaggcgcaattgcgtccaagcttacagacgctagaggcgcactggcgttcatgatggccggggctgcgggaacagctcttggtacatggacatcggtgggttttgtctttgacatgctaggcggctatgctgccgcctcatccactgcttgcttgacatttaaatgcttgatgggtgagtggcccactatggatcagcttgctggtttagtctactccgcgttcaatccggccgcaggagttgtgggcgtcttgtcagcttgtgcaatgtttgctttgacaacagcagggccagatcactggcccaacagacttcttactatgcttgctaggagcaacactgtatgtaatgagtactttattgccactcgtgacatccgcaggaagatactgggcattctggaggcatctaccccctggagtgtcatatcagcttgcatccgttggctccacaccccgacggaggatgattgcggcctcattgcttggggtctagagatttggcagtatgtgtgcaatttctttgtgatttgctttaatgtccttaaagctggagttcagagcatggttaacattcctggttgtcctttctacagctgccagaaggggtacaagggcccctggattggatcaggtatgctccaagcacgctgtccatgcggtgctgaactcatcttttctgttgagaatggttttgcaaaactttacaaaggacccagaacttgttcaaattactggagaggggctgttccagtcaacgctaggctgtgtgggtcggctagaccggacccaactgattggactagtcttgtcgtcaattatggcgttagggactactgtaaatatgagaaaatgggagatcacatttttgttacagcagtatcctctccaaatgtctgtttcacccaggtgcccccaaccttgagagctgcagtggccgtggacggcgtacaggttcagtgttatctaggtgagcccaaaactccttggacgacatctgcttgctgttacggtcctgacggtaagggtaaaactgttaagcttcccttccgcgttgacggtcacacacctggtgtgcgcatgcaacttaatttgcgtgatgcacttgagacaaatgactgtaattccacaaacaacactcctagtgatgaagccgcagtgtccgctcttgttttcaaacaggagttgcggcgtacaaaccaattgcttgaggcaatttcagctggcgttgacaccaccaaactgccagccccctccatcgaagaggtagtggtaagaaagcgccagttccgggcaagaactggttcgcttaccttgcctccccctccgagatccgtcccaggagtgtcatgtcctgaaagcctgcaacgaagtgacccgttagaaggtccttcaaacctccctccttcaccacctgttctacagttggccatgccgatgcccctgttgggagcgggtgagtgtaaccctttcactgcaattggatgtgcaatgaccgaaacaggcggaggccctgatgatttacccagttaccctcccaaaaaggaggtctctgaatggtcagacgaaagttggtcaacggctacaaccgtttccagctacgttactggccccccgtaccctaagatacggggaaaggattccactcagtcagcccccgccaaacggcctacaaaaaagaagttgggaaagagtgagttttcgtgcagcatgagctacacctggaccgacgtgattagcttcaaaactgcttctaaagttctgtctgcaactcgggccatcactagtggtttcctcaaacaaagatcattggtgtatgtgactgagccgcgggatgcggagcttagaaaacaaaaagtcactattaatagacaacctctgttccccccatcataccacaagcaagtgagattggctaaggaaaaagcttcaaaagttgtcggtgtcatgtgggactatgatgaagtagcagctcacacgccctctaagtctgctaagtcccacatcactggccttcggggcactgatgttcgttctggagcagcccgcaaggctgttctggacttgcagaagtgtgtcgaggcaggtgagataccgagtcattatcggcaaactgtgatagttccaaaggaggaggtcttcgtgaagaccccccagaaaccaacaaagaaacccccaaggcttatctcgtacccccaccttgaaatgagatgtgttgagaagatgtactacggtcaggttgctcctgacgtagttaaagctgtcatgggagatgcgtacgggtttgtcgacccacgtacccgtgtcaagcgtctgttgtcgatgtggtcacccgatgcagtcggagccacatgcgatacagtgtgttttgacagtaccatcacacccgaggatatcatggtggagacagacatctactcagcagctaaactcagtgaccaacaccgagctggcattcacaccattgcgaggcagttatacgctggaggaccgatgatcgcttatgatggccgagagatcggatatcgtaggtgtaggtcttccggcgtctatactacctcaagttccaacagtttgacctgctggctgaaggtaaatgctgcagccgaacaggctggcatgaagaaccctcgcttccttatttgcggcgatgattgcaccgtaatttggaagagcgccggagcagatgcagacaaacaagcaatgcgtgtctttgctagctggatgaaggtgatgggtgcaccacaagattgtgtgcctcaacccaaatacagtttggaagaattaacatcatgctcatcaaatgttacctctggaattaccaaaagtggcaagccttactactttcttacaagagatcctcgtatcccccttggcaggtgctctgccgagggtctgggatacaaccccagtgctgcgtggattgggtatctaatacatcactacccatgtttgtgggttagccgtgtgttggctgtccatttcatggagcagatgctctttgaggacaaacttcccgagacggtgacctttgactggtatgggaaaaattatacggtgcctgtagaagatctgcccagcatcattgctggtgtgcacggtattgaggctttctcggtggtgcgctacaccaacgctgagatcctcagagtttcccaatcactaacagacatgaccatgccccccctgcgagcctggcgaaagaaagccagggcggtcctcgccagcgccaagaggcgtggcggagcacacgcaaaattggctcgcttccttctctggcatgctacatctagacctctaccagatttggataagacgagcgtggctcggtacaccactttcaattattgtgatgtttactccccggagggggatgtgtttattacaccacagagaagattgcagaagtttcttgtgaagtatttggctgtcattgtttttgccctagggctcattgctgttggattagccatcagctgaacccccaaattcaaaattaactaacagtttttttttttttttttttttttagggcagcggcaacaggggagaccccgggcttaacgaccccgccgatgtgagtttggcgaccatggtggatcagaaccgtttcgggtgaagccatggtctgaaggggatgacgtcccttctggctcatccacaaaaaccgtctcgggtgggtgaggagtcctggctgtgtgggaagcagtcagtataattcccgtcgtgtgtggtgacgcctcacgacgtatttgtccgctgtgcagagcgtagtaccaagggctgcaccccggtttttgttccaagcggagggcaacccccgcttggaattaaaaactcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacctgaggtaattataacccgggccctatatatggatcccgtaatacgactcactatag

SEQ ID NO: 23 - DNA, 13042 bp, Artificial Sequence; identical to SEQ ID NO: 17
  Description of Artificial Sequence  Synthetic Primer

SEQ ID NO: 24 - DNA, 12265 bp, Artificial Sequence
  Description of Artificial Sequence  Synthetic Primer
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgcctgttatttctactcaaacaagtcctgtacctgcgcccagaacgcgcaagaacaagcagacgcaggcttcatatcctgtgtccattaaaacatctgttgaaaggggacaacgagcaaagcgcaaagtccagcgcgatgctcggcctcgtaattacaaaattgctggtatccatgatggcttgcagacattggctcaggctgctttgccagctcatggttggggacgccaagaccctcgccataagtctcgcaatcttggaatccttctggattaccctttggggtggattggtgatgttacaactcacacacctctagtaggcccgctggtggcaggagcggtcgttcgaccagtctgccagatagtacgcttgctggaggatggagtcaactgggctactggttggttcggtgtccacctttttgtggtatgtctgctatctttggcctgtccctgtagtggggcgcgggtcactgacccagacacaaataccacaatcctgaccaattgctgccagcgtaatcaggttatctattgttctccttccacttgcctacacgagcctggttgtgtgatctgtgcggacgagtgctgggttcccgccaatccgtacatctcacacccttccaattggactggcacggactccttcttggctgaccacattgattttgttatgggcgctcttgtgacctgtgacgcccttgacattggtgagttgtgtggtgcgtgtgtattagtcggtgactggcttgtcaggcactggcttattcacatagacctcaatgaaactggtacttgttacctggaagtgcccactggaatagatcctgggttcctagggtttatcgggtggatggccggcaaggtcgaggctgtcatcttcttgaccaaactggcttcacaagtaccatacgctattgcgactatgtttagcagtgtacactacctggcggttggcgctctgatctactatgcctctcggggcaagtggtatcagttgctcctagcgcttatgctttacatagaagcgacctctggaaaccctatcagggtgcccactggatgctcaatagctgagttttgctcgcctttgatgataccatgtccttgccactcttatttgagtgagaatgtgtcagaagtcatttgttacagtccaaagtggaccaggcctatcactctggagtataacaactccatatcttggtacccctatacaatccctggtgcgaggggatgtatggttaaattcaaaaataacacatggggttgctgccgtattcgcaatgtgccatcgtactgcactatgggcactgatgcagtgtggaacgacactcgcaacacttacgaagtatgcggtgtaacaccatggctaacaaccgcatggcacaacggctcagccctgaaattggctatattacaataccctgggtctaaagaaatgtttaaacctcataattggatgtcaggccatttgtattttgagggatcagatacccctatagtttacttttatgaccctgtgaattccactctcctaccaccggagaggtgggctaggttgcccggtaccccacctgtggtacgtggttcttggttacaggttccgcaagggttttacagtgatgtgaaagacctagccacaggattgatcaccaaagacaaagcctggaaaaattatcaggtcttatattccgccacgggtgctttgtctcttacgggagttaccaccaaggccgtggtgctaattctgttggggttgtgtggcagcaagtatcttattttagcctacctctgttacttgtccctttgttttgggcgcgcttctggttaccctttgcgtcctgtgctcccatcccagtcgtatctccaagctggctgggatgttttgtctaaagctcaagtagctccttttgctttgattttcttcatctgttgctatctccgctgcaggctacgttatgctgcccttttagggtttgtgcccatggctgcgggcttgcccctaactttctttgttgcagcagctgctgcccaaccagattatgactggtgggtgcgactgctagtggcagggttagttttgtgggccggccgtaaccgtggtcaccgcatagctctgcttgtaggtccttggcctctggtagcgcttttaaccctcttgcatttggttacgcctgcttcagctctggacacggaggtggccgcgtcgtgtggcggcgttgttcttgtcgggttaatggcgctgactctgtcgccatattacaagcgctatatcagctggtgcatgtggtggcttcagtattttctgaccagagtagaagcgcaactgcacgtgtgggttccccccctcaacgtccggggggggcgcgatgccgtcatcttactcatgtgtgtagtacacccgaccctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgtcttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgctggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctactgcctttgtgggtgctggcctagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggttggggtaaacactccggcctcttaagccatttcctgttttttttttttttttttttttttttttctttttttttttctttcctttccttctttttttcctttctttttcccttctttaatggtggctccatcttagccctagtcacggctagctgtgaaaggtccgtgagccgcatgactgcagagagtgctgatactggcctctctgcagatcatgttctagactggaattcgtcgacgagctccctatagtgagtcgtattagaggccgacttggccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacaaaatattaacgtttacaatttcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacttggtcagcggccgctaatacgactcactata

SEQ ID NO: 25 - DNA, 13147 bp, Artificial Sequence
  Description of Artificial Sequence  Synthetic Primer
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagccgcgcgggtcactgacccagacacaaataccacaatcctgaccaattgctgccagcgtaatcaggttatctattgttctccttccacttgcctacacgagcctggttgtgtgatctgtgcggacgagtgctgggttcccgccaatccgtacatctcacacccttccaattggactggcacggactccttcttggctgaccacattgattttgttatgggcgctcttgtgacctgtgacgcccttgacattggtgagttgtgtggtgcgtgtgtattagtcggtgactggcttgtcaggcactggcttattcacatagacctcaatgaaactggtacttgttacctggaagtgcccactggaatagatcctgggttcctagggtttatcgggtggatggccggcaaggtcgaggctgtcatcttcttgaccaaactggcttcacaagtaccatacgctattgcgactatgtttagcagtgtacactacctggcggttggcgctctgatctactatgcctctcggggcaagtggtatcagttgctcctagcgcttatgctttacatagaagcgacctctggaaaccctatcagggtgcccactggatgctcaatagctgagttttgctcgcctttgatgataccatgtccttgccactcttatttgagtgagaatgtgtcagaagtcatttgttacagtccaaagtggaccaggcctatcactctggagtataacaactccatatcttggtacccctatacaatccctggtgcgaggggatgtatggttaaattcaaaaataacacatggggttgctgccgtattcgcaatgtgccatcgtactgcactatgggcactgatgcagtgtggaacgacactcgcaacacttacgaagtatgcggtgtaacaccatggctaacaaccgcatggcacaacggctcagccctgaaattggctatattacaataccctgggtctaaagaaatgtttaaacctcataattggatgtcaggccatttgtattttgagggatcagatacccctatagtttacttttatgaccctgtgaattccactctcctaccaccggagaggtgggctaggttgcccggtaccccacctgtggtacgtggttcttggttacaggttccgcaagggttttacagtgatgtgaaagacctagccacaggattgatcaccaaagacaaagcctggaaaaattatcaggtcttatattccgccacgggtgctttgtctcttacgggagttaccaccaaggccgtggtgctaattctgttggggttgtgtggcagcaagtatcttattttagcctacctctgttacttgtccctttgttttgggcgcgcttctggttaccctttgcgtcctgtgctcccatcccagtcgtatctccaagctggctgggatgttttgtctaaagctcaagtagctccttttgctttgattttcttcatctgttgctatctccgctgcaggctacgttatgctgcccttttagggtttgtgcccatggctgcgggcttgcccctaactttctttgttgcagcagctgctgcccaaccagattatgactggtgggtgcgactgctagtggcagggttagttttgtgggccggccgtaaccgtggtcaccgcatagctctgcttgtaggtccttggcctctggtagcgcttttaaccctcttgcatttggttacgcctgcttcagcttttgataccgagataattggagggctgacaataccacctgtagtagcattagttgtcatgtctcgttttggcttctttgctcacttgttacctcgctgtgctttagttaactcctatctttggcaacgttgggagaattggttttggaacgttacactaagaccggagaggtttttccttgtgctggtttgtttccccggtgcgacatatgacgcgctggtgactttctgtgtgtgtcacgtagctcttctatgtttaacatccagtgcagcatcgttctttgggactgactctagggttagggcccatagaatgttggtgcgtctcggaaagtgtcatgcttggtattctcattatgttcttaagtttttcctcttagtgtttggtgagaatggtgtgtttttctataagcacttgcatggtgatgtcttgcctaatgattttgcctcgaaactaccattgcaagagccatttttcccttttgaaggcaaggcaagggtctataggaatgaaggaagacgcttggcgtgtggggacacggttgatggtttgcccgttgttgcgcgtctcggcgaccttgttttcgcagggttggctatgccgccagatgggtgggccattaccgcaccttttacgctgcagtgtctctctgaacgtggcacgctgtcagcgatggcagtggtcatgactggtatagacccccgaacttggactggaactatcttcagattaggatctctggccactagctacatgggattcgtttgtgacaacgtgttgtatactgctcaccatggcagcaaggggcgccggttggctcatcccacaggctccatacacccaataaccgttgacgcggctaatgaccaggacatctatcaaccaccatgtggagctgggtcccttactcggtgctcttgcggggagaccaaggggtatctggtaacacgactggggtcattggttgaggtcaacaaatccgatgacccttattggtgtgtgtgcggggcccttcccatggctgttgccaagggttcttcaggtgccccgattctgtgctcctccgggcatgttattgggatgttcaccgctgctagaaattctggcggttcagtcagtcagattagggttaggccgttggtgtgtgctggataccatccccagtacacagcacatgccactcttgatacaaaaacgcgtatgcagatcttcgtgaagaccctgacgggcaagaccatcactcttgaggtcgagcccagtgacaccatcgagaatgtcaaggccaagatccaagacaaggaaggcatcccacctgaccagcagaggctgatattcgcgggcaaacagctggaggatggccgcaccctgtccgactacaacatccagaaagagtccaccttgcacctggtgctgcgtctccgcggtggcgcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctactgcctttgtgggtgctggcctagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggttggggtaaacactccggcctcttaagccatttcctgttttttttttttttttttttttttttttctttttttttttctttcctttccttctttttttcctttctttttcccttctttaatggtggctccatcttagccctagtcacggctagctgtgaaaggtccgtgagccgcatgactgcagagagtgctgatactggcctctctgcagatcatgttctagactggaattcgtcgacgagctccctatagtgagtcgtattagaggccgacttggccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacaaaatattaacgtttacaatttcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacttggtcagcggccgctaatacgactcactata

SEQ ID NO: 26 - DNA, 12370 bp, Artificial Sequence
  Description of Artificial Sequence  Synthetic Primer
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagccgcgcgggtcactgacccagacacaaataccacaatcctgaccaattgctgccagcgtaatcaggttatctattgttctccttccacttgcctacacgagcctggttgtgtgatctgtgcggacgagtgctgggttcccgccaatccgtacatctcacacccttccaattggactggcacggactccttcttggctgaccacattgattttgttatgggcgctcttgtgacctgtgacgcccttgacattggtgagttgtgtggtgcgtgtgtattagtcggtgactggcttgtcaggcactggcttattcacatagacctcaatgaaactggtacttgttacctggaagtgcccactggaatagatcctgggttcctagggtttatcgggtggatggccggcaaggtcgaggctgtcatcttcttgaccaaactggcttcacaagtaccatacgctattgcgactatgtttagcagtgtacactacctggcggttggcgctctgatctactatgcctctcggggcaagtggtatcagttgctcctagcgcttatgctttacatagaagcgacctctggaaaccctatcagggtgcccactggatgctcaatagctgagttttgctcgcctttgatgataccatgtccttgccactcttatttgagtgagaatgtgtcagaagtcatttgttacagtccaaagtggaccaggcctatcactctggagtataacaactccatatcttggtacccctatacaatccctggtgcgaggggatgtatggttaaattcaaaaataacacatggggttgctgccgtattcgcaatgtgccatcgtactgcactatgggcactgatgcagtgtggaacgacactcgcaacacttacgaagtatgcggtgtaacaccatggctaacaaccgcatggcacaacggctcagccctgaaattggctatattacaataccctgggtctaaagaaatgtttaaacctcataattggatgtcaggccatttgtattttgagggatcagatacccctatagtttacttttatgaccctgtgaattccactctcctaccaccggagaggtgggctaggttgcccggtaccccacctgtggtacgtggttcttggttacaggttccgcaagggttttacagtgatgtgaaagacctagccacaggattgatcaccaaagacaaagcctggaaaaattatcaggtcttatattccgccacgggtgctttgtctcttacgggagttaccaccaaggccgtggtgctaattctgttggggttgtgtggcagcaagtatcttattttagcctacctctgttacttgtccctttgttttgggcgcgcttctggttaccctttgcgtcctgtgctcccatcccagtcgtatctccaagctggctgggatgttttgtctaaagctcaagtagctccttttgctttgattttcttcatctgttgctatctccgctgcaggctacgttatgctgcccttttagggtttgtgcccatggctgcgggcttgcccctaactttctttgttgcagcagctgctgcccaaccagattatgactggtgggtgcgactgctagtggcagggttagttttgtgggccggccgtaaccgtggtcaccgcatagctctgcttgtaggtccttggcctctggtagcgcttttaaccctcttgcatttggttacgcctgcttcagctctggacacggaggtggccgcgtcgtgtggcggcgttgttcttgtcgggttaatggcgctgactctgtcgccatattacaagcgctatatcagctggtgcatgtggtggcttcagtattttctgaccagagtagaagcgcaactgcacgtgtgggttccccccctcaacgtccggggggggcgcgatgccgtcatcttactcatgtgtgtagtacacccgaccctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgtcttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgctggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctactgcctttgtgggtgctggcctagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggttggggtaaacactccggcctcttaagccatttcctgttttttttttttttttttttttttttttctttttttttttctttcctttccttctttttttcctttctttttcccttctttaatggtggctccatcttagccctagtcacggctagctgtgaaaggtccgtgagccgcatgactgcagagagtgctgatactggcctctctgcagatcatgttctagactggaattcgtcgacgagctccctatagtgagtcgtattagaggccgacttggccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacaaaatattaacgtttacaatttcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacttggtcagcggccgctaatacgactcactata

SEQ ID NO: 27 - DNA, 27 bp, Artificial Sequence
  Description of Artificial Sequence  Synthetic Primer
aattcggccctgcaggccacaacagtc

(19) United States
(12) Patent Application Publication
(10) Pub. No.: US 2006/0105365 A1
(43) Pub. Date: May 18, 2006

(54) Chimeric GB virus B (GBV-B)

(76) Inventors: Annette Martin, Paris (FR); David Ghibaudo, Saint Apollinaire (FR); Lisette Cohen, Le Kremlin Bicetre (FR); Stanley M. Lemon, Galveston, TX (US)

Correspondence Address: FULBRIGHT & JAWORSKI L.L.P., 600 CONGRESS AVE., SUITE 2400, AUSTIN, TX, 78701, US

(21) Appl. No.: 11/236,836

(22) Filed: Sep. 27, 2005

Related U.S. Application Data
(60) Provisional application No. 60/613,266, filed on Sep. 27, 2004.

(51) Int. Cl. C12Q 1/70; C12Q 1/68

(52) U.S. Cl. 435/6; 435/5

(57) ABSTRACT

The present invention relates generally to the fields of biochemistry, molecular biology, and virology. More particularly, it relates to the production and use of GB virus B (GBV-B)/HCV chimeras. The invention involves nucleic acid constructs and compositions encoding GBV-B/HCV chimera. The chimeric viruses may be employed to study GBV-B and related hepatitis family members, such as hepatitis C virus. The invention thus includes methods of preparing GBV-B/HCV chimeric sequences, constructs, and viruses, as well as methods of employing these compositions.

This application claims priority to U.S. Provisional Patent Application Ser. No. 60/613,266, filed Sep. 27, 2004, which is incorporated herein by reference in its entirety.

### BACKGROUND OF THE INVENTION

A. Field of the Invention

The present invention relates generally to the fields of biochemistry, molecular biology, and virology. More particularly, it relates to compositions and methods related to GBVirus-B (GBV-B)/Hepatitis C virus (HCV) chimeric viruses, polynucleotides, and proteins.

B. Description of Related Art

Chronic hepatitis C is a major threat to the public health. Serologic surveys suggest that as many as 3.9 million Americans are chronically infected with the responsible virus, hepatitis C virus (HCV) (Alter, 1997). These individuals are at increased risk of developing progressive hepatic fibrosis leading to cirrhosis and loss of hepatocellular function, as well as hepatocellular carcinoma. The course of chronic hepatitis C is typically lengthy, often extending over decades, with insidious clinical progression usually occurring in the absence of symptoms. Nonetheless, liver disease due to HCV results in the death of 8,000-10,000 Americans annually, and chronic hepatitis C is the most common cause of liver transplantation within the U.S.

Therefore, HCV is a major public health problem. However, therapy for chronic hepatitis C is problematic. Recombinant interferon-α is approved for treatment of chronic hepatitis C (Consensus Development Panel, 1997). The benefit of interferon-α results primarily from its antiviral properties and its ability to inhibit production of virus by infected hepatocytes (Neumann et al., 1998). Nonetheless, even under optimal therapeutic regimens, the majority of patients with chronic hepatitis C fail to eliminate the virus or resolve their liver disease. Treatment failures are especially common in persons infected with genotype 1 HCV, unfortunately the most prevalent genotype in the U.S. Thus, there is an urgent need to better understand the virus and develop better treatment. Unfortunately, technical difficulties in working with HCV have made it necessary to use infectious surrogate viruses in efforts to develop treatments and vaccines for HCV.

Scientists' efforts to better understand HCV and to develop new drugs for treatment of hepatitis C have been stymied by two overwhelming technical deficiencies: first, the nonexistence of a high permissive cell line that supports replication of the virus and second, the absence of a permissive animal species other than chimpanzees, which are endangered and therefore available on a limited basis.

Presently, those who are working on HCV treatment and prevention are employing an infectious chimeric virus of sindbis and HCV and/or an infectious clone of pestiviruses as surrogate virus models in HCV drug discovery efforts, due to the above technical difficulties of working with HCV. Alternatively, they are using isolated proteins or RNA segments of HCV for biochemical and structural studies. This approach precludes functional studies of virus replication and its inhibition.

GBV-B is a hepatotropic flavivirus that has a unique phylogenetic relationship to human HCV and strong potential to serve as a surrogate virus in drug discovery efforts related to hepatitis C antiviral drug development. GBV-B causes acute hepatitis in experimentally infected tamarins (Simons et al., 1995; Schlauder et al., 1995; Karayiannis et al., 1989) and can serve as a surrogate virus for HCV in drug discovery efforts. GBV-B virus is much closer in sequence and biological properties than the above-described models. It will be easier to make biologically relevant chimeras between HCV and GBV-B than by using more distantly related viruses. GBV-B is hepatotropic (as is HCV), whereas the viruses used in these competing technologies are not. In view of the above, an infectious clone of GBV-B would be useful to those working on HCV treatment and prevention.

Unfortunately, the use of GBV-B as a surrogate or model for HCV has not been possible in the past, because no infectious molecular clone of GBV-B virus genome could be prepared. It is now known that this obstacle was encountered because the GBV-B genome was believed to be 259 nucleotides shorter than its actual length (Muerhoff et al., 1995; Simons et al., 1995). Others, previous to the inventors, had failed to realize that the 3′ sequence of GBV-B was missing from the prior sequences. Without this 3′ sequence, it is not possible to prepare an infectious GBV-B molecular clone.

### BRIEF SUMMARY OF THE INVENTION

As discussed above, an infectious molecular clone of GBV-B or GBV-B/HCV chimera would be very useful for the development of HCV preventative and therapeutic treatments. In particular, chimeric viruses, polynucleotides, and/or proteins are used as compositions and in the methods of the exemplified invention. The construction of an infectious molecular clone may require the newly determined 3′ sequence to be included in order for the clone to be viable. The inventors have elucidated the previously unrecognized 3′ terminal sequence of GBV-B (SEQ ID NO:1). This sequence has been reproducibly recovered from tamarin serum containing GBV-B RNA, in RT-PCR protocols using several different primer sets, and as a fusion with previously reported 5′ GBV-B sequences. The newly identified 3′ sequence is not included in published reports of the GBV-B sequence, nor described in patents relating to the original identification of the viral sequence (see U.S. Pat. No. 5,807,670 and references therein). In various embodiments of the invention an infectious clone involving the 3′ terminal sequence from GVB-B may or may not be required.

The invention has utility in that the inclusion of the sequence may be necessary, if desired, to construct an infectious molecular GBV-B clone. Such clones clearly have the potential to be constructed as chimeras including relevant hepatitis C virus sequences in lieu of the homologous GBV-B sequence, providing unique tools for drug discovery efforts. A full-length molecular clone of GBV- was constructed, as described in later sections of this specification.

GBV-B can be used as a model for HCV, and the GBV-B genome, polynucleotides, and/or polypeptides can be used in the construction of chimeric viral RNAs, DNAs and/or polypeptides containing sequences of both HCV and GBV-B. Such chimeric viruses or molecules enable the investigation of the mechanisms for the different biological properties of these viruses and encoded proteins, and to discover and investigate potential inhibitors of specific HCV activities (e.g., proteinase) required for HCV replication. However, certain aspects of this work is dependent upon construction of an infectious clone of GBV-B, which is itself dependent on the incorporation of the correct 3′ terminal nucleotide sequence within this clone. GBV-B has unique advantages over HCV in terms of its ability to replicate and cause liver disease in tamarins, which present fewer restrictions to research than chimpanzees, the only nonhuman primate species known to be permissive for HCV.

Embodiments of the invention include a chimeric GBV-B/HCV virus, a chimeric polynucleotide and/or an encoded chimeric polypeptide thereof. In certain aspects, the invention is an isolated polynucleotide encoding a chimeric GBV-B/HCV virus (i.e., a virus containing GBV-B nucleic acid sequences with one or more corresponding HCV nucleic acid sequences replacing GBV-B sequences), chimeric GBV-B/HCV polynucleotide (e.g., a nucleic acid containing GVB-B sequences and all or, part of a nucleic acid segment encoding an HCV core region), and/or chimeric GBV-B/HCV polypeptide (e.g., all or part of a chimeric core protein). It will be understood that a cognizable sequence (nucleotide or amino acid) from GBV-B or HCV refers to a sequence that can be recognized as from one or the other. “A corresponding sequence” refers to a sequence that corresponds in number and/or homology to the replaced sequence.

Typically, segment(s) of GBV-B genome, polynucleotide and/or encoded polypeptide are replaced by a corresponding segment(s) of HCV. In some embodiments of a chimeric virus or polynucleotide, for example, the virus or polynucleotide contains in addition to GBV-B sequences, an HCV nucleic acid segment encoding all or part of one or more of the following HCV proteins: a core protein; an E1 protein; an E2 protein; a p7 protein; an E1 and E2 protein; a core, E1, and E2 proteins; a core, E1, E2, and p7 proteins; NS2; NS3; NS4; NS4A; NS4b; NS5A; NS5B and various combinations and/or permutations thereof and may further contain a protease site such as, but not limited to Ubi. In further aspects, the polynucleotide of the invention may encode a polyprotein with one or more heterologous protease sites. Embodiments cover 1, 2, 3, 4, 5, 6, 7, 8, 9, 10, 11, 12, 13, 14, 15, or more protease sites that may be recognized by the same or difference proteases. A protease site, such as a Ubi site, may be located between protein regions or within a leader sequence of a protein, such as any of those discussed above or herein. For example, a NS3 protein may have a heterologous protease cleavage site and in particular an ubiquitin protease cleavage site.

A polynucleotide and/or polypeptide of the invention may comprise, comprise at least, or comprise at most 1, 2, 3, 4, 5, 6, 7, 8, 9, 10, 15, 20, 25, 30, 35, 40, 45, 50, 55, 60, 65, 70, 75, 80, 85, 90, 95, 96, 97 98, 99%, or any value derivable therefrom of an HCV genome, nucleic acid sequence or amino acid sequence. In certain non-limiting embodiments, the polynucleotide may have a sequence set forth in SEQ ID NO:17, SEQ ID NO:18, SEQ ID NO:19, SEQ ID NO:20, SEQ ID NO:21, SEQ ID NO:22, SEQ ID NO:24, or SEQ ID NO:26. The polynucleotide may be a DNA or an RNA. The polynucleotide of the invention may be comprised in a plasmid.

In further embodiments the invention includes a hepatotropic virus. The hepatotropic virus may be a chimeric GBV-B/HCV virus as exemplified herein. Typically the hepatotropic virus will propagate in vivo. In particular aspects the chimeric virus will propagate in a primate or primate cells. In a preferred embodiment, the chimeric virus will propagate in a tamarin or cells derived there from.

In still a further embodiment of the invention includes a method of producing a chimeric virus. The method may include a) introducing into a host cell a viral expression construct comprising a polynucleotide encoding a chimeric GBV-B/HCV virus; and b) culturing said host cell under conditions permitting production of a chimeric virus from the construct. A host cell may be a prokaryotic cell, a eukaryotic cell, an animal, and more preferably a tamarin. The polynucleotide may comprise synthetic or recombinant RNA or DNA. The method may further comprising the step of isolating virus from a host or host cell. Preferably the virus is purified to homogeneity.

Other embodiments of the invention include methods for identifying or assessing the effectiveness a compound active against a viral infection comprising: a) providing a virus, polynucleotide, and/or polypeptide expressed from an expression construct comprising a chimeric GBV-B/HCV virus or polynucleotide; b) contacting the virus, polynucleotide, and/or polypeptide with a candidate substance; and c) comparing or assessing the infectious ability of the virus or effect on a polynucleotide or polypeptide in the presence of the candidate substance with the infectious ability of the virus or the characteristics of a polynucleotide or polypeptide in a similar system in the absence of the candidate substance.

In addition, an infectious molecular clone of GBV-B or a GVB-B/HCV chimera is expected to have utility in liver-specific gene expression or in gene therapy. This application might be enhanced by the inclusion of HCV genomic sequence. Further, an infectious GBV-B/HCV chimera expressing HCV polypeptides (e.g., envelope proteins) can have utility as a vaccine immunogen for hepatitis C.

A full-length cDNA copy of the GBV-B genome or a GBV-B/HCV chimera may be constructed to contain the newly identified 3′ terminal sequences. RNA transcribed from this cDNA copy of the genome would be infectious when inoculated into the liver of a GBV-B permissive tamarin, giving rise to rescued GBV-B or GBV-B/HCV virus particles. A chimeric molecule may be constructed from this infectious GBV-B clone in which all or part of the HCV 5′ NTR, C region sequence or encoded protein, E1 region sequence or encoded protein, E2 region sequence or encoded protein, p7 region sequence or encoded protein, NS2 region sequence or encoded protein, NS3 region sequence or encoded protein, NS4A region sequence or encoded protein, NS4B region sequence or encoded protein, NS5A region sequence or encoded protein, NS5B region sequence or encoded protein, 3′ NTR or various combinations and permutations would be placed in frame or in an operative position in lieu of the homologous GBV-B sequence, and this chimeric cDNA would be used to generate infectious GBV-B/HCV chimeric viruses by intrahepatic inoculation of synthetic RNA in tamarins. Published studies indicate that the GBV-B and HCV proteinases have closely related substrate recognition and cleavage properties, making such chimeras highly likely to be viable. These newly generated chimeric GBV-B/HCV viruses could be used in preclinical assessment of candidate HCV NS3 proteinase inhibitors as well as molecules that alter various characteristics of other polynucleotides or polypeptides of HCV.

Therefore, aspects of the present invention encompass an isolated polynucleotide encoding a 3′ sequence of the GBV-B genome. The polynucleotide may include the sequence identified as SEQ ID NO:1. It is contemplated that the polynucleotide may be a DNA molecule or it can be an RNA molecule. It is further contemplated that expression constructs may contain a polynucleotide that has a stretch of contiguous nucleotides from SEQ ID NO:1 and/or SEQ ID NO:2, for example, lengths of 50, 100, 150, 250, 500, 1000, 5000, as well as the entire length of SEQ ID NO:1 or 2, are considered appropriate. Such polynucleotides may also be contained in other constructs of the invention or be used in the methods of the invention. Polynucleotides, such as chimeric GBV-B/HCV polynucleotides, employing sequences from SEQ ID NO:1 may alternatively contain sequences from SEQ ID NO:2 in the constructs and methods of the present invention.

The invention is also understood as covering a viral expression construct that includes a polynucleotide encoding a 3′ sequence of the GBV-B genome. This expression construct is further understood to contain the sequence identified as SEQ ID NO:1. The present invention contemplates the expression construct as a plasmid or as a virus. Furthermore, the expression construct can express GBV-B sequences; alternatively it may express sequences from a chimeric GBV-B/HCV virus.

The identification and isolation of a 3′ sequence of GBV-B additionally provides a method of producing a virus, particularly a full-length virus, by introducing into a host cell an expression construct containing a polynucleotide encoding at least a 3′ sequence of GBV-B and by culturing the host cell under conditions permitting production of a virus from the construct. This method can be practiced using a prokaryotic cell as a host cell, or by using a eukaryotic cell as a host cell. Furthermore, the eukaryotic cell can be located within an animal.

A method of producing virus according to the claimed invention can also be employed using a polynucleotide that contains synthetic RNA and/or synthetic DNA. Moreover, a step can be added to the method by also isolating any virus produced from the host cell. The virus can then be purified to homogeneity.

Additional examples of the claimed invention include a method for identifying a compound active against a viral infection by providing a virus, polynucleotide, and/or polypeptide expressed from an expression construct, which may or may not contain a 3′ sequence of a GBV-B virus, by contacting the virus, polynucleotide, and/or polypeptide with a candidate substance; and by comparing the infectious ability of the virus or function of the polynucleotide or polypeptide in the presence of the candidate substance with the infectious ability or function in a similar system in the absence of the candidate substance. It is contemplated that the invention can be practiced using GBV-B virus or a GBV-B/HCV chimera or various segments thereof, including nucleic acid or peptide segement(s).

The present invention can also be understood to provide a compound active against a viral infection, e.g., HCV infection, identified by providing a virus, polynucleotide, and/or polypeptide expressed from a viral construct containing a 3′ sequence of a GBV-B virus; contacting the virus, polynucleotide, and/or polypeptide with a candidate substance; and comparing the infectious ability of the virus or the function of the polynucleotide or polypeptide in the presence of the candidate substance with a similar system in the absence of the candidate substance. In some embodiments an active compound is identified using a GBV-B virus, while in other embodiments an active compound is identified using a GBV-B/HCV chimera or polynucleotide or polypeptide segments thereof.

In various embodiments of the invention, a GBV-B polynucleotide may encode a GBV-B/HCV chimera that includes at least part of a 5′ NTR sequence derived from a HCV 5′ NTR. The 5′ NTR may comprise at least one domain derived from the 5′ NTR of HCV. In certain embodiments, the GVB-B/HCV chimera may include at least domain III of the 5′ NTR derived from the 5′NTR of HCV. In yet other embodiments the infectious GBV-B clone may comprise domain III of the 5′ NTR of HCV, which may or may not include one or more structural or non-structural genes of HCV also incorporated into the chimeric virus. The portions of the 5′ NTR of the GVB-B/HCV chimeras will generally be replaced by analogous sequences from the 5′ NTR of HCV. It will be understood that the portions or parts of the 5′ NTR of GBV-B that may be replaced include all or part of domain I (including sub-region Ia and Ib of GBV-B), domain II, domain III, domain IV, or any combination thereof. Any combination of 5′ NTR domains of GBV-B may be replaced with an analogous region of HCV. In certain embodiments, the replacement of a GBV-B region may be accompanied by the deletion of the 5′ NTR GBV-B domain Ib region. In addition, any one, two, or three of the 5′ NTR domains of GBV-B may be replaced in any combination with analogous sequences from HCV.

In further embodiments of the invention, a polynucleotide encoding a GBV-B/HCV chimera including a 5′ NTR domain III sequence derived from a HCV 5′ NTR may be propagated in vivo, in particular, in the liver of an appropriate host.

Various other embodiments may include isolated polynucleotides comprising a chimeric GBV-B genome, wherein at least part, but not all of a 5′ NTR sequence is derived from a HCV 5′ NTR. The polynucleotides may be synthetic RNA, RNA, DNA or the like.

Some embodiments include one or more virus, one or more hepatotropic virus, and/or one or more viral expression constructs comprising a chimeric GBV-B polynucleotides including at least a part of the 5′ NTR sequence is derived from a HCV 5′ NTR.

Methods of producing a chimeric GBV-B virus encoding at least part of a 5′ NTR sequence derived from a HCV 5′ NTR sequence comprising introducing into a host cell a viral expression construct comprising a chimeric GBV-B polynucleotide encoding at least part of a 5′ NTR sequence derived from a HCV 5′ NTR sequence and culturing said host cell under conditions permitting production of a virus from said construct are contemplated. The method may use a host cell that is a eukaryotic cell and the host cell a may in an animal. The method may further include the step of isolating virus from said host cell and in particular purify the virus to homogeneity.

In addition, methods for identifying a compound active against a viral infection comprising are contemplated. The methods may include providing a virus expressed from a viral construct comprising at least part of a 5′ NTR derived from a HCV 5′ NTR, as described herein; contacting said virus with a candidate substance; and comparing the infectious ability of the virus in the presence of said candidate substance with the infectious ability of the virus in a similar system in the absence of said candidate substance. Each of the embodiments may use or include any of the 5′ NTR chimeras described herein.

It is contemplated that any method or composition described herein can be implemented with respect to any other method or composition described herein.

Other embodiments of the invention may include a compound active against a viral infection identified according to the method described above.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

It is specifically contemplated that any embodiments described in the Examples section are included as an embodiment of the invention.

As used herein the specification, “a” or “an” may mean one or more. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Schematic representation of full-length, chimeric GBV-B/HCV cDNAs containing substitution of (C)-E1-E2-p7/p13 coding sequences. cDNA sequences from GBV-B and HCV are represented by blue and grey boxes, respectively, and the corresponding nontranslated regions (NTR) and encoded proteins are indicated on top of or below each genome. The boundaries of the substituted sequences encoding E1-E2-p7/p13 or C-E1-E2-p7/p13 are indicated by their respective amino acid positions within parental polyproteins for each chimeric GBV-B/HCV cDNA constructed (as named on the left). Restriction sites that have been used to linearize cDNAs prior to in vitro transcription are positioned within the cDNAs and indicated by arrows.

FIG. 2. Schematic representation of full-length, chimeric GBV-B/HCV cDNAs containing substitution of (C)-E1-E2-p7/p13-NS2 coding sequences. cDNA sequences from GBV-B and HCV are represented by blue and grey boxes, respectively, and the corresponding nontranslated regions (NTR) and encoded proteins are indicated on top of each genome. In each chimeric GBV-B/HCV cDNA (as named on the left), the substituted sequences encoding E1-E2-p7/p13-NS2 or C-E1-E2-p7/p13-NS2 are C-terminally fused to sequences of the protease domain of NS3 (NS3pro) derived from the same virus, followed by the ubiquitin gene (Ubi, red box). The location of the cleavage carried out by the deubiquitination enzyme is indicated by a dark red arrow. Restriction sites that have been used to linearize cDNAs prior to in vitro transcription are positioned within the cDNAs and indicated by arrows.

FIGS. 3A and 3B. Comparative analysis of translational efficiencies of chimeric RNAs containing heterologous 5′nontranslated region (NTR) and core-coding sequences with chimeric RNAs containing homologous such sequences. (3A) Schematic representation of RNAs transcribed in vitro from chimeric cDNAs linearized at the BamHI or AvrII restriction sites in the HCV or GBV-B backbones, respectively. (3B) Chimeric, truncated RNAs (as indicated on the top of the gel) were translated in vitro in rabbit reticulocyte lysates in the presence of [35S]Met and the resulting products were separated by SDS-PAGE. Polypeptides are marked by a star (as in panel A) at the right of each lane in the gel, and identified on either side of the gel. The number of methionine residues (# Met) present in each polypeptide is indicated at the bottom of the gel for quantitation purposes (see text).

FIGS. 4A and 4B. Analysis of the signal peptidase-mediated proteolytic cleavages at heterologous junctions within chimeric polyproteins containing substitutions of (C)-E1-E2-p7/p13 sequences. Panel (4A) shows data relative to the heterologous C/E1 junction, whereas panel (4B) shows data relative to the heterologous p13/NS2 junction. In panel (4A), RNAs were transcribed from parental or chimeric cDNAs linearized within the N-terminal NS3 coding sequence at the BstZ17I restriction site in HCV or the AflIII site in GBV-B (see FIG. 1). In panel (4B), RNAs were transcribed from cDNAs linearized either within the NS4B coding sequence, or within the NS3 coding sequence (site BstZ17I), as indicated below the gel. Corresponding, encoded polypeptide precursors are schematically represented at the bottom of each panel. RNAs were translated in rabbit reticulocyte lysates in the presence of canine microsomal membranes and either [35S]Cys (4A) or [35S]Met (4B). Resulting polypeptides were separated by 16% (4A) or 14% (4B) SDS-PAGE. GBV-B (marked “GB”) and HCV (marked “HC”) proteins are identified on both sides of the gels.

FIGS. 5A and 5B. Analysis of the signal peptidase-mediated proteolytic cleavages at heterologous junctions within chimeric polyproteins containing substitutions of E1-E2-p7/p13-NS2 sequences. (5A) Schematic representation of chimeric polypeptide precursors translated from RNAs transcribed in vitro from cDNAs linearized at the indicated restriction sites (see also FIG. 2). Cleavages carried out by either cellular signal peptidases (⋄), viral NS2-3 proteinase (arrow), or viral NS3/4A proteinase (arrow) within these polypeptides are indicated. NS3pro represents the N-terminal third of NS3 that corresponds to the protease domain of NS3. (5B) RNAs transcribed from cDNA templates indicated above each lane of the gels were translated in rabbit reticulocyte lysates in the presence of [35S]Met, and in the presence (left panel) or in the absence (right panel) of canine microsomal membranes. Resulting polypeptides were separated by 14% SDS-PAGE. GBV-B (marked as “GB”) and HCV (marked as “HC”) polypeptides, as well as fusion polypeptides with ubiquitin (Ubi) are idientified on both sides of the gels. Proteins with identical electrophoresis mobilities (NS3HC, E2HC, NS3GB) are indicated by special signs at the right side of each lane.

FIGS. 6A and 6B. Analysis of the replication capacities in cell culture of chimeric genomes generated in the backbone of HCV A. cDNAs from either infectious genotype 1a HCV (Yanagi et al., 1997), cell culture-adapted genotype 1a HCV (HCV A; Yi and Lemon, 2004), chimeric HCA/C-p13GB or HCA/E1-p13GB constructs in the backbone of HCV A (A), or chimeric HCA/C-NS3pro GB-Ubi or HCA/E1-NS3pro GB-Ubi constructs in the backbone of HCV A (B), were linearized at the XbaI restriction site prior to in vitro transcription to generate corresponding full-length RNAs. 2×10⁶ Huh-7 cells were transfected with 5 μg of each RNA by electroporation (240 volts, 900 μF). Non-adapted, infectious genotype 1a HCV RNA, which is not capable of replication in cell culture, was used as a negative control. Total RNA was extracted from transfected cells at 4 days post-transfection, quantified by optical density, and 7.5 μg of these RNAs were loaded on a denaturing agarose gel, transferred to a Nylon membrane, and subjected to detection by Northern blot with an [alpha-32P]-UTP-labeled riboprobe of negative polarity specific for HCV 3′ sequences. Known quantities of in vitro transcribed viral HCV A RNAs (10⁷ à 10⁹ genome equivalents (ge)) were mixed with RNAs isolated from mock-transfected cells and run in parallel on the gel to serve as a size marker and for quantitation purposes. The amount of viral RNAs present in each extract was quantified by densitometry after analysis of the Northern blots with a PhophorImager (Molecular Dynamics) and normalized with respect to amounts of housekeeping beta-actin mRNAs present in the same samples and quantified with an [a-32P]-UTP-labeled specific riboprobe (bottom images). Cellular and viral RNAs of interest are identified by arrows.

FIGS. 7A-7D. Identification of chimeric virus-like particles by electron microscopy. Cellular extracts from insect cells infected with recombinant baculoviruses expressing parental C-p7HC (7A, 7B) or chimeric CGB/E1-p7HC (7C, 7D) structural protein precursors were prepared and fractionated on sucrose gradients to isolate virus-like particles (VLPs). After immunobloting with specific antibodies directed to all three structural proteins (Core of HCV or GBV-B, E1 and E2 of HCV), 2 fractions (#12+13) were found to contain CHC (7A, 7B) or CGB (7C, 7D), as well as E1HC and E2HC. These fractions were pooled, diluted in PBS, and centrifugated to eliminate sucrose and concentrate the immuno-reactive material. After immunogold labeling with antibodies specific for E1HC (A4; panels 7B, 7D), or antibodies specific for E2HC that recognize a functionally-folded form of E2 (H53; panels 7A, 7C), followed by staining with uranyl acetate, observation of the material by electron microscopy revealed the presence of VLPs with both parental (7A, 7B) and chimeric (7C, 7D) constructs. Scale bars at the bottom of the pictures correspond to 100 nm.

FIG. 8 Shows cDNA of chimeric constructs of the invention.

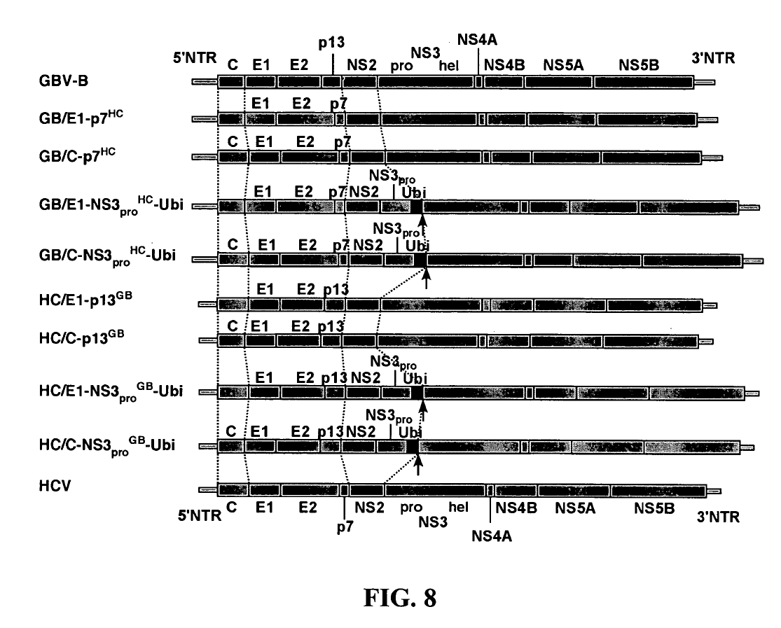

### DESCRIPTION OF ILLUSTRATIVE EMBODIMENTS

I. GVB-B Virus

The GBV-B genome structure is very similar to hepatitis C and these viruses share approximately 25% nucleotide identity (Simons et al., 1995; Muerhoff et al., 1995). As indicated above, this makes GBV-B more closely related to HCV than any other known virus. GBV-B genomic RNA is about 9.5 kb in length (Muerhoff et al., 1995) with a structured 5′ noncoding region that contains an IRES that shares many structural features with the HCV IRES (Honda et al., 1996; Rijnbrand et al., 1999). As in HCV, this IRES drives the cap-independent translation of a long open reading frame. The polyprotein expressed from this reading frame appears to be organized identically to that of HCV, and processed to generate proteins with functions similar to those of HCV (Muerhoff et al., 1995). In fact, the major serine proteinases of these viruses (NS3) have been shown to have similar cleavage specificities (Scarselli et al., 1997). Finally, like HCV and distinct from the pestiviruses, the genomic RNA of GBV-B has a poly(U) tract located near its 3′ terminus (Simons et al., 1995; Muerhoff et al., 1995). In addition, unreported sequences located at the extreme 3′ end of the genome have been identified. This work indicates that the GBV-B RNA, like that of HCV (HCV (Tanaka et al., 1995; Kolykhalov et al., 1996), terminates in a lengthy run of heterogeneous bases (310 nts in GBV-B) possessing a readily apparent secondary structure.

The HCV structural region (C E1 E2 p7) polypeptide contains four internally located endoplasmic reticulum (ER) signal peptide sequences, which lead the nascent HCV polyprotein to translocate across the ER membrane. Host signal peptidases cleave the signal peptides at their C terminus in the lumen of the ER and release the mature viral proteins, including the capsid protein C, the two envelope glycoproteins E1 and E2, and the p7 polypeptide, immediately upstream of sequences of NS2 nonstructural protein. Signal peptides remain N terminally linked to the C terminus of E1, E2, and p7, and are likely to serve as an ER membrane anchor for those proteins, due to the hydrophobicity of their amino acid sequence (Op De Beeck et al., 2001). In contrast, the signal peptide present at the C terminus of the core protein is cleaved off in the mature form of the core protein (McLauchlan et al., 2002).

The hydropathy profile of the N terminal part of the GBV B polyprotein is very similar to that of the HCV structural precursor. The inventors have determined by sequencing the boundaries of the GBV-B core, E1, E2, and NS2 proteins and demonstrated the existence of a 13 kDa-protein between E2 and NS2 that is partially homologous to HCV p7 (Ghibaudo et al., 2004).

Studies using transient viral and non-viral expression systems have shown that HCV glycoproteins can follow two different folding pathways, leading to the formation of either noncovalent heterodimers, the probable native complexes, or disulfide bond aggregates, likely representing dead-end products. The folding of E1 is slow and co-expression of E2 is necessary for the proper folding of E1. In addition, transmembrane domains of both E1 and E2 are involved in heterodimerization and contain E2 retention signals. The native E1-E2 complexes identified in the ER most probably represent a prebudding form of HCV glycoprotein heterodimers (Dubuisson, 2000).

HCV p7 is a small transmembrane protein that functions as an ion channel (Carrere-Kremer et al., 2002; Griffin et al., 2003) and that has been shown to be necessary in the virus life cycle (Sakai et al., 2003). GBV-B p13 is likely to share ion channel acitivity, but this remains presently unexplored.

The HCV and GBV-B NS2 sequences are located downstream of p7 and p13 sequences, respectively, and are cleaved at their N terminus by a cellular signal peptidase, as mentioned. These proteins are very hydrophobic and contain several putative transmembrane domains (Yamaga and Ou, 2002). HCV NS2 is not required for RNA replication, as it has been shown that subgenomic replicons containing only the NS3 to NS5B sequences of HCV, hence devoid of the NS2 sequence, are capable of replication in Huh7 cells (Lohmann et al., 1999). The only known function of HCV NS2 is to carry a proteolytic activity that is responsible for cis-cleavage at the NS2/NS3 junction (Grakoui et al., 1993; Pieroni et al., 1997). The exact nature of this proteolytic activity is still controversial, and it is not known whether it is a metalloproteinase or a cysteine protease. Because only the C-terminal half of NS2 is required for the NS2/NS3 cleavage, it is very likely that NS2 plays another, yet undefined but critical role in the viral life cycle. There is a strong possibility that it may play a role in infectious viral particle assembly and/or particle final maturation steps and export, based on analogy with other flaviviruses (Kummerer and Rice, 2002; Agapov et al., 2004). The mechanisms underlying HCV particle assembly, budding and release remain, however, very poorly understood in the absence of a cell culture system that supports HCV replication.

One goal was to create chimeric GBV-B/HCV genomes that encode GBV-B sequences necessary for tamarin host-range specificity and HCV sequences substituted to GBV-B sequences that are not specifically required for virus replication in tamarins. Since GBV-B host-range determinants are unknown, chimeric cDNAs were constructed in which the replication unit, comprising sequences encoding all nonstructural proteins as well as 5′ and 3′ nontranslated regions (NTRs), was derived from GBV-B, and sequences encoding envelope glycoproteins were derived from HCV. Similarly, complementary genomes designed to encode GBV B envelope proteins in an HCV backbone were also constructed.

In designing such genomes, it was considered likely that proper glycoprotein folding and particle assembly would require the two homologous E1 and E2 proteins of GBV-B, like with HCV. Therefore, sequences encoding both E1 and E2 were considered as a unit in the substitutions described herein. In the absence of information on the virus-specific role of p7/p13 proteins, sequences of these proteins were also exchanged together with that of E1-E2 proteins.

Second, whether there are virus-specific interactions between core (C) protein and the viral RNA or between core and envelope (E1, E2) proteins is unknown for these flaviviruses. Only one report described a specific interaction between HCV C and E1 proteins (Lo et al., 1996). One piece of evidence suggesting that core protein/RNA interaction may be more stringent than core/envelope protein interaction comes from the fact that it is possible to substitute envelope proteins (prM-E) only, but not capsid and envelope proteins together (C-prM-E), between viruses of the flavivirus genus without disrupting genome infectivity (Pletnev and Men, 1998; Chambers et al., 1999). Third, whether cellular signalases would be able to process heterologous junctions that were created in the chimeric polyproteins are assessed.

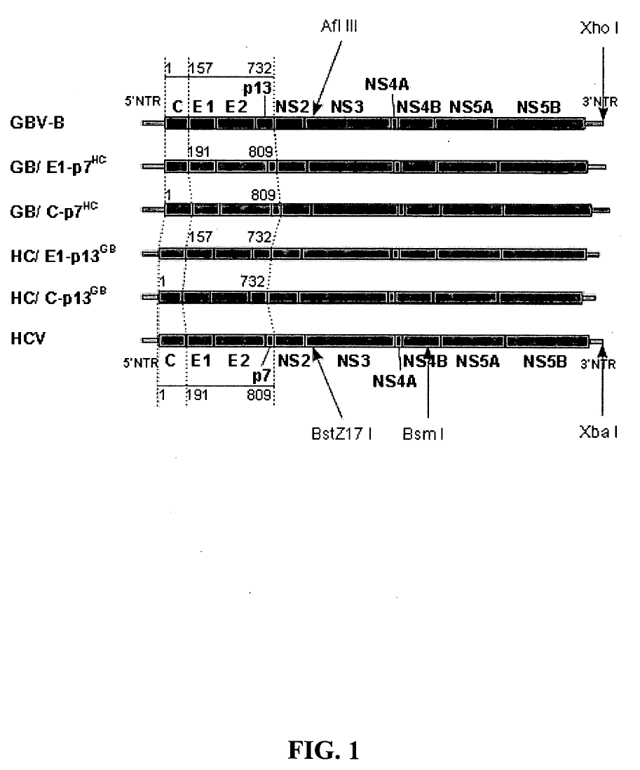

Chimeric cDNAs were engineered in which the sequences encoding E1-E2-p13 only or C-E1-E2-p13 were exchanged. For that purpose, a mutagenesis strategy based on overlapping extension PCR was used. Two chimeric cDNAs were thus obtained in the backbone of an infectious GBV-B molecular clone (Martin et al., 2003), in which E1-E2-p13 or C-E1-E2-p13 sequences were replaced by E1-E2-p7 or C-E1-E2-p7 sequences of the H77 strain of HCV gentoype 1a (Yanagi et al., 1997) (GB/E1-p7HC, GB/C-p7HC; FIG. 1). GBV-B protein boundaries were derived from protein sequencing (Ghibaudo et al., 2004). Complementary constructs containing GBV-B E1-E2-p13 or C-E1-E2-p13 sequences in the backbone of the infectious HCV 1a molecular clone (gift of R. Purcell, N.I.A.I.D., Bethesda) were also engineered (HC/E1-p13GB, HC/C-p13GB; FIG. 1).

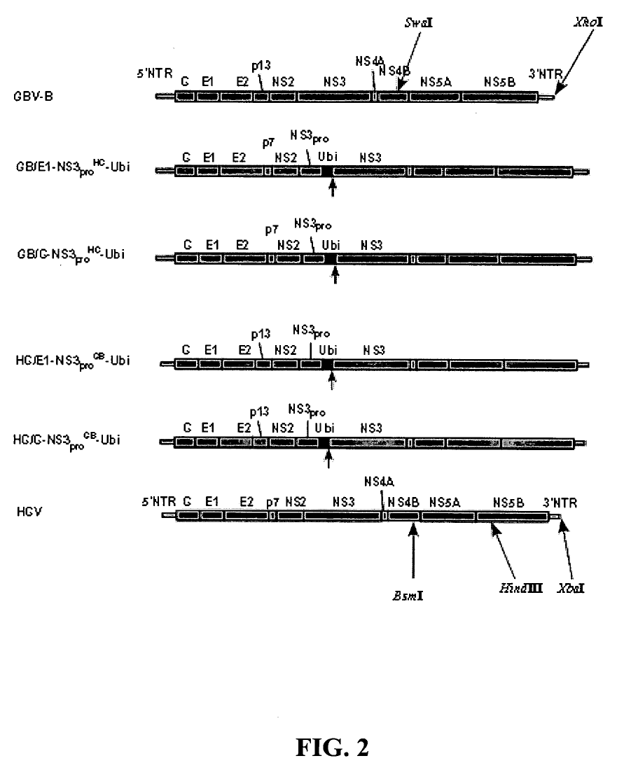

Chimeric GBV-B or HCV cDNAs were also engineered in which sequences encoding E1-E2-p7/p13-NS2 were replaced by analogous sequences from the other virus. To avoid problems at the fusion site between heterologous NS2 and NS3 sequences, the sequence of the ubiquitin gene may be inserted such as to cleave the chimeric polyprotein at the first amino acid of the downstream NS3 sequence. To avoid fusion of the ubiquitin sequence to the upstream heterologous NS2 protein and to release mature NS2, sequences of the proteinase domain of NS3 from the same virus, which are known to be sufficient for cis-cleavage at the NS2/NS3 site in HCV (Thibeault et al., 2001), were fused to the NS2 sequences. Hence, chimeric GBV-B cDNAs pGB/E-NS3proHC-Ubi or pGB/C-NS3proHC-Ubi encoding (C)-E1-E2-p7-NS2-NS3pro proteins of HCV followed by Ubiquitin, as well as chimeric HCV cDNAs pHC/E1-NS3proGB-Ubi or pHC/C-NS3proHC-Ubi encoding (C)-E1-E2-p13-NS2-NS3pro proteins of GBV-B followed by Ubiquitin were generated (FIG. 2).

II. Nucleic Acids

The present invention provides a nucleic acid sequence encoding a 3′ sequence of the GBV-B genome (SEQ ID NO:1). It should be clear that the present invention is not limited to the specific nucleic acids disclosed herein. As discussed below, a “3′ sequence of the GBV-B genome” may contain a variety of different bases and yet still be functionally indistinguishable from the sequences disclosed herein.

A. Polynucleotides Encoding the 3′ Sequence of the GBV-V Genome

A 3′ sequence of the GBV-B genome disclosed in SEQ ID NO:1 is one aspect of the present invention. Nucleic acids according to the present invention may encode the 3′ sequence of the GBV-B genome set forth in SEQ ID NO:1, the entire GBV-B genome, or any other fragment of a 3′ sequence of the GBV-B genome set forth herein. The nucleic acid may be derived from genomic RNA as cDNA, i.e., cloned directly from the genome of GBV-B. cDNA may also be assembled from synthetic oligonucleotide segments.

It also is contemplated that a 3′ sequence of the GBV-B genome may be represented by natural variants that have slightly different nucleic acid sequences but, nonetheless, maintain the same general structure and perform the same function in RNA replication.

As used in this application, the term “a nucleic acid encoding a 3′ sequence of the GBV-B genome” refers to a nucleic acid molecule that may be isolated free of total viral nucleic acid. In preferred embodiments, the invention concerns nucleic acid sequences essentially as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12. The term “as set forth in SEQ ID NO:1” means that the nucleic acid sequence substantially corresponds to a portion of SEQ ID NO:1. It is contemplated that the techniques and methods described in this disclosure may apply to any of the sequences contained herein, including SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of SEQ ID NO:1 will be sequences that are “as set forth in SEQ ID NO:1.” Sequences that are essentially the same as those set forth in SEQ ID NO:1 may also be functionally defined as sequences that are capable of hybridizing to a nucleic acid segment containing the complement of SEQ ID NO:1 under standard conditions.

The nucleic acid segments and polynucleotides of the present invention include those encoding biologically functional equivalent 3′ sequences of the GBV-B genome. Changes designed by man may be introduced through the application of site-directed mutagenesis techniques or may be introduced randomly and screened later for the desired function, as described below.

3′ sequence of the GBV-B genome sequences also are provided. Each of the foregoing is included within all aspects of the following description. The present invention concerns cDNA segments reverse transcribed from GBV-B genomic RNA (referred to as “DNA”). As used herein, the term “polynucleotide” refers to an RNA or DNA molecule that may be isolated free of other RNA or DNA of a particular species.

“Isolated substantially away from other coding sequences” means that the 3′ sequence of the GBV-B genome forms the significant part of the RNA or DNA segment and that the segment does not contain large portions of naturally-occurring coding RNA or DNA, such as large fragments or other functional genes or cDNA noncoding regions. Of course, this refers to the polynucleotide as originally isolated, and does not exclude genes or coding regions later added to the it by the hand of man.

In certain other embodiments, the invention concerns isolated DNA segments (cDNA segments reverse transcribed from GVB-B genomic RNA) and recombinant vectors that include within their sequence a nucleic acid sequence essentially as set forth in SEQ ID NO:1. The term “essentially as set forth in SEQ ID NO:1” is used in the same sense as described above.

It also will be understood that nucleic acid sequences may include additional residues, such as additional 5′ or 3′ sequences, and still be essentially as set forth in one of the sequences disclosed herein, so long as the sequence meets the criteria set forth above, including the maintenance of biological activity. The addition of terminal sequences particularly applies to nucleic acid sequences that may, for example, include additional various non-coding sequences flanking either of the 5′ or 3′ portions of the coding region, which are known to occur within viral genomes.

Sequences that are essentially the same as those set forth in SEQ ID NO:1 also may be functionally defined as sequences that are capable of hybridizing to a nucleic acid segment containing the complement of SEQ ID NO:1 under relatively stringent conditions. Suitable relatively stringent hybridization conditions will be well known to those of skill in the art.

The nucleic acid segments of the present invention, regardless of the length of the coding sequence itself, may be combined with other RNA or DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

For example, nucleic acid fragments may be prepared that include a short contiguous stretch identical to or complementary to SEQ ID NO:1, such as about 15-24 or about 25-34 nucleotides and that are up to about 259 nucleotides being preferred in certain cases. Other stretches of contiguous sequence that may be identical or complementary to any of the sequences disclosed herein, including the SEQ ID NOS. include the following ranges of nucleotides: 50-9,399, 100-9,000, 150-8,000, 200-7,000, 250-6,000, 300-5,000, 350-4,000, 400-3,000, 450-2,000, 500-1000. RNA and DNA segments with total lengths of about 1,000, about 500, about 200, about 100 and about 50 base pairs in length (including all intermediate lengths) are also contemplated to be useful.

In a non-limiting example, one or more nucleic acid constructs may be prepared that include a contiguous stretch of nucleotides identical to or complementary to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, or SEQ ID NO:12. Such a stretch of nucleotides, or a nucleic acid construct, may be about, or at least about, 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39 about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 618, about 650, about 700, about 750, about 1,000, about 2,000, about 3,000, about 4,000, about 5,000, about 6,000, about 7,000, about 8,000, about 9,000, about 9,100, about 9,200, about 9,300, about 9,399, about 9,400, about 9,500, about 9,600, about 9,700, about 9,800, about 9,900, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art.

It will be readily understood that “intermediate lengths,” in these contexts means any length between the quoted ranges, such as 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, etc.; 100, 101, 102, 103, etc.; 150, 151, 152, 153, etc.; including all integers through the 200-500; 500-1,000; 1,000-2,000; ranges, up to and including sequences of about 1,001, 1,250, 1,500, and the like.

The various probes and primers designed around the disclosed nucleotide sequences of the present invention may be of any length. By assigning numeric values to a sequence, for example, the first residue is 1, the second residue is 2, etc., an algorithm defining all primers can be proposed:
n to n+y

where n is an integer from 1 to the last number of the sequence and y is the length of the primer minus one, where n+y does not exceed the last number of the sequence. Thus, for a 20-mer, the probes correspond to bases 1 to 20, 2 to 21, 3 to 22 . . . and so on. For a 30-mer, the probes correspond to bases 1 to 30, 2 to 31, 3 to 32 . . . and so on. For a 35-mer, the probes correspond to bases 1 to 35, 2 to 36, 3 to 37 . . . and so on.

B. Oligonucleotide Probes and Primers

Naturally, the present invention also encompasses RNA and DNA segments that are complementary, or essentially complementary, to the sequence set forth in SEQ ID NO:1. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO. 1 under relatively stringent conditions such as those described herein. Such sequences may encode the entire 3′ sequence of the GBV-B genome or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 225 or more base pairs will be used, although others are contemplated. Longer polynucleotides encoding 250, 500, 1000, 1212, 1500, 2000, 2500, 3000 or 3431 bases and longer are contemplated as well. Such oligonucleotides will find use, for example, as probes in Southern and Northern blots and as primers in amplification reactions.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C. Formamide and SDS also may be used to alter the hybridization conditions.

One method of using probes and primers of the present invention is in the search for other viral sequences related to GBV-B or, more particularly, homologs of the GBV-B sequence. By varying the stringency of hybridization, and the region of the probe, different degrees of homology may be discovered.

Another way of exploiting probes and primers of the present invention is in site-directed, or site-specific, mutagenesis. The technique provides a ready ability to prepare and test sequence variants, incorporating one or more of the foregoing considerations, by introducing one or more nucleotide sequence changes into complementary DNA. Site-specific mutagenesis allows the production of mutants through the use of specific oligonucleotide sequences that encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement. There are newer and simpler site-directed mutagenesis techniques that can also be employed for this purpose. These include procedures marketed in kit form that are readily available to one of ordinary skill in the art.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

C. Antisense Constructs

In certain embodiments of the invention, the use of antisense constructs of the 3′ sequence of the GBV-B genome is contemplated.

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, may be employed to inhibit gene transcription or translation or both within a host cell, either in vitro or in vivo, such as within a host animal, including a human subject.

Antisense constructs could be used to block early steps in the replication of GBV-B and related viruses, by annealing to 3′ terminal sequences and blocking their role in negative-strand initiation.

As stated above, “complementary” or “antisense” means polynucleotide sequences that are substantially complementary over their entire length and have very few base mismatches. For example, sequences of 15 bases in length may be termed complementary when they have complementary nucleotides at 13 or 14 positions. Naturally, sequences which are completely complementary will be sequences which are entirely complementary throughout their entire length and have no base mismatches. Other sequences with lower degrees of homology also are contemplated. For example, an antisense construct which has limited regions of high homology but also contains a non-homologous region (e.g., ribozyme; see below) could be designed. These molecules, though having less than 50% homology, would bind to target sequences under appropriate conditions.

D. Amplification and PCR™

The present invention utilizes amplification techniques in a number of its embodiments. Nucleic acids used as a template for amplification are isolated from cells contained in the biological sample, according to standard methodologies (Sambrook et al., 1989). The nucleic acid may be genomic DNA or RNA or fractionated or whole cell RNA. Where RNA is used, it may be desired to convert the RNA to a complementary DNA using reverse transcriptase (RT). In one embodiment, the RNA is genomic RNA and is used directly as the template for amplification. In others, genomic RNA is first converted to a complementary DNA sequence (cDNA) and this product is amplified according to protocols described below.

Pairs of primers that selectively hybridize to nucleic acids corresponding to GBV-B sequences are contacted with the isolated nucleic acid under conditions that permit selective hybridization. The term “primer,” as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred.

Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced.

Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals.

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and each incorporated herein by reference in entirety.

Briefly, in PCR™, two or more primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al., 1989. Alternative methods for reverse transcription utilize thermostable, RNA-dependent DNA polymerases. These methods are described in WO 90/07641, filed Dec. 21, 1990, incorporated herein by reference. Polymerase chain reaction methodologies are well known in the art.

Another method for amplification is the ligase chain reaction (“LCR”), disclosed in EPA No. 320 308, incorporated herein by reference in its entirety. U.S. Pat. No. 4,883,750 describes a method similar to LCR for binding probe pairs to a target sequence.

Qbeta Replicase, described in PCT Application No. PCT/US87/00880, incorporated herein by reference, also may be used as still another amplification method in the present invention.

An isothermal amplification method, in which restriction endonucleases and ligases are used to achieve the amplification of target molecules that contain nucleotide 5′-[alpha-thio]-triphosphates in one strand of a restriction site also may be useful in the amplification of nucleic acids in the present invention.

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences also can be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR Gingeras et al., PCT Application WO 88/10315, incorporated herein by reference.

Davey et al., EPA No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention.

Miller et al., PCT Application WO 89/06700 (incorporated herein by reference in its entirety) disclose a nucleic acid sequence amplification scheme based on the hybridization of a promoter/primer sequence to a target ssDNA followed by transcription of many RNA copies of the sequence. This scheme is not cyclic, i.e., new templates are not produced from the resultant RNA transcripts. Other amplification methods include “RACE” and “one-sided PCR” (Frohman, 1990 incorporated by reference).

Methods based on ligation of two (or more) oligonucleotides in the presence of nucleic acid having the sequence of the resulting “di-oligonucleotide”, thereby amplifying the di-oligonucleotide, also may be used in the amplification step of the present invention.

Following any amplification, it may be desirable to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography that may be used in the present invention: adsorption, partition, ion-exchange and molecular sieve, and many specialized techniques for using them including column, paper, thin-layer and gas chromatography.

Amplification products must be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by Southern blotting and hybridization with a labeled probe. The techniques involved in Southern blotting are well known to those of skill in the art and can be found in many standard books on molecular protocols. See Sambrook et al., 1989. Briefly, amplification products are separated by gel electrophoresis. The gel is then contacted with a membrane, such as nitrocellulose or nylon, permitting transfer of the nucleic acid and non-covalent binding. Subsequently, the membrane is incubated with a chromophore-conjugated probe that is capable of hybridizing with a target amplification product. Detection is by exposure of the membrane to x-ray film or ion-emitting detection devices.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

E. Expression Constructs

In some embodiments of the present invention, an expression construct that encodes a 3′ sequence of GBV-B is utilized. The term “expression construct” is meant to include any type of genetic construct containing a nucleic acid coding for a gene product in which part or all of the nucleic acid encoding sequence is capable of being transcribed. The transcript may be translated into a protein, but it need not be. Expression includes both transcription of a gene and translation of mRNA into a gene product. Expression may also include only transcription of the nucleic acid encoding a gene of interest.

In some constructs, the nucleic acid encoding a gene product is under transcriptional control of promoter and/or enhancer. The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies have shown that promoters are composed of discrete functional modules, each consisting of approximately 7-20 bp of nucleic acids, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of nucleic acids with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

F. Host Cells and Permissive Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of the present invention, “host cell” refers to a prokaryotic or eukaryotic cell, and it includes any transformable organisms that is capable of replicating a vector or virus and/or expressing viral proteins. A host cell can, and has been, used as a recipient for vectors, including viral vectors. A host cell may be “transfected” or “transformed,” which refers to a process by which exogenous nucleic acid is transferred or introduced into the host cell. A transformed cell includes the primary subject cell and its progeny. A “permissive cell” refers to a cell that supports the replication of a given virus and consequently undergoes cell lysis. In the context of the present invention, such a virus would include HCV, GBV-B, or other hepatitis viruses. In a “nonpermissive cell,” productive infection does not result, but the cell may become stably transformed. In some embodiments, methods employ permissive cells that are a cell line derived from liver cells (liver cell line).

Host cells may be derived from prokaryotes or eukaryotes, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector or virus or virus particle may be used in conjunction with either a eukaryotic or prokaryotic host cell, particularly one that is permissive for replication or expression of the vector. It is contemplated that the present invention includes vectors composed of viral sequences, viruses, and viral particles in the methods of the present invention, and that they may be used interchangeably in these methods, depending on their utility.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

III. Pharmaceutical Compositions

The present invention encompasses the use of a 3′ sequence of GBV-B in the production of or use as a vaccine to combat HCV infection. Compositions of the present invention comprise an effective amount of GBV-B clone as a therapeutic dissolved or dispersed in a pharmaceutically acceptable carrier or aqueous medium. The phrases “pharmaceutically or pharmacologically acceptable” refer to molecular entities and compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

The biological material should be extensively dialyzed to remove undesired small molecular weight molecules and/or lyophilized for more ready formulation into a desired vehicle, where appropriate. The active compounds will then generally be formulated for parenteral administration, e.g., formulated for injection via the intravenous, intramuscular, sub-cutaneous, intralesional, or even intraperitoneal routes. The preparation of an aqueous composition that contains GVB-B nucleic acid sequences as an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

A GBV-B clone of the present invention can be formulated into a composition in a neutral or salt form. Pharmaceutically acceptable salts include the acid addition salts (formed with the free amino groups of the protein) and are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. In terms of using peptide therapeutics as active ingredients, the technology of U.S. Pat. Nos. 4,608,251; 4,601,903; 4,599,231; 4,599,230; 4,596,792; and 4,578,770, each incorporated herein by reference, may be used.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like also can be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous or intramuscular injection, other pharmaceutically acceptable forms include, e.g., tablets or other solids for oral administration; liposomal formulations; time release capsules; and any other form currently used, including cremes.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Infectious GBV-B Genome

The inventors have elucidated a previously unrecognized 3′ terminal sequence of GBV-B (SEQ ID NO 1). This sequence was reproducibly recovered from tamarin serum containing GBV-B RNA, in RT-PCR nucleic acid amplification procedures using several different primer sets, and as a fusion with previously reported 5′ GBV-B sequences.

There is information in the published literature reporting the putative sequences of the 5′ and 3′ termini of the GBV-B genome. The nucleic acid sequences of these termini were reportedly determined by ligating the ends of the viral RNA together, amplifying the sequence in the region of the resulting junction by reverse-transcription polymerase chain reaction (RT-PCR), and sequencing of the cDNA amplification product across the junction. However, the inventors believed these results required confirmation. Of particular concern was the fact that the 3′ terminus appeared to be shorter than the equivalent region of other viruses in the family Flaviviridae (especially within the genus Hepacivirus) and that the reported 3′ sequence lacked a defined RNA hairpin structure such as those present in these related viruses. Additional novel sequences at the 3′ end of the GBV-B genome were investigated using a serum sample collected from a tamarin that was experimentally infected with virus. Amplification was used to determine the sequence of the 3′ end.

First, serum (50 μL) known to contain GBV-B RNA by RT-PCR assay was extracted with Trizol, and the RNA was washed and dried. A synthetic oligonucleotide was then ligated to the 3′ end of the viral RNA. The oligonucleotide, AATTCGGCCCTGCAGGCCACAACAGTC (SEQ ID NO:27), which was phosphorylated at the 5′ end and chemically blocked at the 3′ end, was ligated to the RNA essentially using the method described by Kolykhalov et al. (1996). The RNA was initially dissolved in DMSO and the following additions were made: Tris-Cl, pH 7.5 (10 mM), MgCl₂ (10 mM), DTT (5 mM), hexamine cobalt chloride (1 mM), 10 pmol oligo and 8 U T4 ligase. The final concentration of DMSO was 30% in a final volume of 10 μL. The ligation reaction was incubated for 4 or 20 hours at 19° C. 1 μL of the ligation reaction was used directly to make cDNA, using a primer complementary to the ligated oligonucleotide and the Superscript 2 system, in a final volume of 15 μL. 1 μL of cDNA was amplified using the Advantage cDNA system (Clontech) and two additional oligonucleotide primers. These primers included one that was complementary to the ligated oligonucleotide (i.e., “negative sense”) and a positive-sense primer located near the 3′ end of the reported GBV-B sequence. A product approximately 290 bases in length was obtained, and this was gel purified and directly sequenced. Sequencing was done in both directions using the oligonucleotide primers employed for the amplification; 259 bases that had not been previously reported were identified as fused to the sequence that had been previously described as the 3′ terminus of the viral genome.

To ensure that this novel 3′ sequence from viral RNA could be reproducibly amplified, an additional 10 μL of infected tamarin serum was extracted using Trizol. cDNA was prepared by reverse transcription using an oligonucleotide primer complementary to the penultimate 3′ 25 bases of the novel sequence. Amplification was then done by PCR using the primer previously utilized for cDNA synthesis and a positive-sense primer mapping within the previously published GBV-B sequence. In the initial studies, although a product was readily detected, DNA sequencing showed that this product was missing all of the sequence distal to the poly-U tract. Carrying out the cDNA synthesis in the presence of DMSO circumvented this problem. A cDNA product of approximately 290 bases was obtained. This was sequenced and shown to consist of the 5′ primer, 20 bases of the published GBV-B sequence, and 259 bases of the novel sequence obtained in the preceding studies and containing the sequence of the 3′ primer. The sequence of the 3′ end of GBV-B is shown in SEQ ID NO:1. The presence of a predicted hairpin structure at the extreme 3′ end of this novel sequence is consistent with its location at the 3′ terminus of the viral RNA.

The GBV-B cDNA (synthesis described above) was used as a template for PCR amplification of the 3′ 1553 nucleotides (nts) of the GBV-B genome. This PCR amplification product was gel purified and cloned into plasmid DNA using the “Perfectly Blunt Cloning Kit” (Novagene).

Construction of an Infectious GBV-B Clone—The elucidation of a 3′ sequence of the GBV-B genome will allow those of skill in the art to construct and validate an infectious molecular clone of GBV-B. This will be done using the following procedures.

A full-length cDNA copy of the GBV-B genome containing the newly identified 3′ terminal sequences was constructed. RNA transcribed from this cDNA copy of the genome will be infectious when inoculated into the liver of a GBV-B permissive tamarin, giving rise to rescued GBV-B virus particles.

A 1:1000 dilution of GBV-B infectious tamarin serum was obtained. This material was used as a source of viral RNA for the amplification of GBV-B nucleic acid sequences by reverse-transcription polymerase chain reaction. For amplification of previously reported segments of the GBV-B genome, 250 μL of the diluted serum was extracted with Trizol using the manufacturer's instructions. The final RNA pellet was dissolved in 10 μL of a 100 mM DTT buffer containing 5% RNasin. This material was converted into cDNA using Superscript 2 reverse transcriptase and oligonucleotide primers designed to be complementary to the reported GBV-B RNA sequence and to contain unique restriction sites. This cDNA was amplified using the Advantage cDNA kit (Clontech) employing the cDNA primer (negative sensiv as the downstream primer and a similar positive-sense upstream primer, again containing a unique restriction site. The published sequence of GBV-B allowed for the selection of primers in convenient areas of the genome containing unique restriction sites. Using this general strategy, the inventors amplified segments of the reported GBV-B genome representing: (1) nucleotides (nts) 1-1988, using an upstream primer containing a T7 RNA polymerase promoter and a BamHI site upstream of nt 1, and a downstream primer containing a unique Eco1R1 site (nt 1978); (2) nts 1968-5337, using a downstream primer containing a unique Cla1 site at position 5o27; (3) nts 5317-7837, using a downstream primer containing a Sal1 site at nt 7847; and, (4) nts 7837-9143, using a downstream primer containing an added Xho1 site. It was found necessary to use different PCR conditions for each primer set.

The RT-PCR products generated in these reactions were cloned into plasmid DNA after gel purification, using the “Perfectly Blunt Cloning Kit” (Novagene). Ten bacterial colonies from each of the four RT-PCR products were analyzed for insert size by restriction endonuclease digestion using EcoR1, the sites for this enzyme being located on either side of the insert in the resulting plasmids. For three of the RT-PCR amplicons, 9 of 10 colonies contained plasmids with the correct size insert. The EcoR1-Cla1 amplicon generated only 1/10 colonies with a correct size insert. Thus, 30 additional colonies were examined, yielding two more clones with insert of the correct size. For each of these plasmids, simple restriction patterns were obtained using two restriction enzymes. As these appeared to be correct, the plasmid DNAs were subjected to sequencing using an ABI automatic sequencer.

Nucleotide sequence of the cloned GBV-B cDNA—The 5′ region of the cloned sequence revealed a relatively long nontranslated region corresponding to the published sequence of the GBV-B 5′NTR, which includes an IRES. This region was followed by a long open reading frame. Near the 3′ end of the genome a poly-U tract was identified; however, this was shorter than the published 3′ homopolymeric poly-U region. The sequence from these clones was compared with those in the GenBank database (Accession U22304, “Hepatitis GB virus B polypeptide complete genome”). Twenty-two nucleotide differences were identified, of which 14 gave rise to amino acid changes (Table 1). In order to determine whether these changes were genuine or RT-PCR artifacts, which could have been introduced due to the very small amount of material from which these sequences were amplified, segments of the genome containing these changes were reamplified using a serum sample from an independently infected tamarin. Of the 14 changes noted in the original cDNA clones, 12 were not present in these newly amplified sequences and thus were probably RT-PCR artifacts (Table 2.). A particularly interesting difference from the published GenBank sequence, however, which was present in both the original clones as well as a repeat amplification, was a two-nucleotide substitution that obliterated the Sal1 site present in the published sequence.

TABLE 1
Differences in the amino acid sequences of GBV-B cDNA clones
and the GBV-B sequence reported by Simons et al. (1995).

| | AMINO ACID Δ | RT-PCR Products |
|---|---|---|
| GBV-B | FROM ABBOTT | from Tamarin 12024 |
| Protein | SEQUENCE | (PCR Reaction #) |
| Core | G₉₉→S | G (38.1a) |
| E1 | V₃₉₅→I | V (40.2a) |
| E2 | D₇₀₃→N | D (42.3a) |
| E2 | P₇₀₆→Q | H (42.3a) |
| E2 | A₇₂₈→V | A (42.3a) |
| NS2 | L₇₉₁→F | F (42.3a) |
| NS2 | T₈₀₄→A | A (42.3a) |
| NS5A | L₁₉₉₀→M | L (46.5a) |
| NS5A | I₂₀₈₂→T | I (46.5a) |
| NS5A | S₂₁₇₄→P | (not done) |
| NS5A | G₂₂₂₈→E | E (48.6a) |
| NS5A | T₂₂₃₃→S | T (48.6a) |
| NS5A | A₂₂₃₆→V | A (48.6a) |
| NS5B | V₂₈₃₃→I | V (50.7a) |

Construction of a full-length GBV-B cDNA clone.—The four GBV-B cDNA inserts described above were cloned into Bluescript ks+ using unique restriction sites. Since the unique SalI site that was reported to be present in the published GBV-B sequence (nt position 7847) was absent in these cDNA clones, this restriction site was created by engineering two silent nucleotide changes using the “Quick Change” mutagenesis system (Stratagene). Although the most 5′ clones (nucleotides 1-7847) could be readily constructed, attempts to add the remaining 3′ clones were unsuccessful due to rearrangements and deletions. This problem was overcome by use of pACNR1180, a plasmid that had been used to construct an infectious clone of yellow fever virus. Finally, the most 3′ 771 nucleotides of GBV-B were excised from the plasmid containing the novel, previously unreported 3′ sequence, and inserted into the truncated assembled GBV-B cDNA construct to complete the 3′ end. The 3′ terminus of this full-length cDNA was then subjected to DNA sequencing to confirm its integrity. Extensive restriction digests indicated that this construct had the characteristics of a full-length c DNA copy of GBV-B virus. Because there is not yet an understanding of which cultured cells (if any) might be permissive for GBV-B replication, the infectivity of the synthetic GBV-B RNA will be assessed by injecting the RNA directly into the liver of a susceptible tamarin.

Alternatively, an infectious full-length clone can be produced by the following protocol. A plasmid will be made containing a cassette including the 5′ and 3′ ends of the virus flanked by appropriate restriction sites. These constructs have been shown to efficiently translate reporter genes, with transcription taking place via a T7 promoter placed immediately upstream of the 5′NTR (e.g., see Rijnbrand et al., 1999). The major portion of the GBV-B genome would then be amplified by long range RT-PCR. This method is now well established for hepatitis C virus and other flaviviruses (Teller et al., 1996), and it has been used successfully also to amplify rhinovirus RNA. Briefly this technique uses “Superscript” reverse transcriptase to synthesize cDNA and a mixture of “KlenTaq 1”, and “DeepVent” polymerases to amplify this cDNA. Primers that can be used will contain restriction sites to allow cloning of the RT-PCR products into the cassette vector. After being transformed into suitable competent bacteria, extensive restriction analysis will enable us to determine which clones contain inserts that are of full length and which have a high probability of being correct. Apparent full-length clones will be analyzed further by coupled transcription-translation using the Promega “TnT” system, with the addition of microsomal membranes to allow the cleavage of the structural proteins by cellular signalase enzymes. Clones which appear correct by restriction analysis and which produce GBV-B proteins, in particular the protein coded for by the extreme 3′ end of the genome, NS5B, will be selected, and RNA will be transcribed from these clones using the Ambion MegaScript system. “Correct” looking clones (>10) can be injected directly into a tamarin liver at several sites. A successful infection will be determined as described below. If a positive signal is detected the entire genome will be amplified and sequenced to determine which plasmid the virus originated from.

Rescue of Infectious GVB-B. Infectious GBV-B will be rescued from synthetic genome-length RNA following its injection into the liver of tamarins (Saquinus sp.). In past studies, HAV from synthetic RNA in owl monkeys has been recovered (Aotus trivirgatus) (Shaffer et al., 1995), and more recently, the recovery of virus from a chimpanzee injected intrahepatically with RNA transcribed from a full-length genotype 1b HCV cDNA clone was reported (Beard et al., 1999).

RNA will be prepared for these studies using the T7 MegaScript kit (Ambion) and a total of 10 μg of plasmid DNA as template. An aliquot of the reaction products will be utilized to ensure the integrity of the RNA by electrophoresis in agarose-formaldehyde gels. The remainder of the transcription reaction mix will be frozen at −80° C. until its injection, without further purification, into the liver of a tamarin. Because of the small size of the tamarin, the RNA will be injected under direct visualization following a limited incision and exposure of the liver. Under similar conditions, in other primate species, RT-PCR-detectable viral RNA or cDNA has not been detected in serum samples collected within days of this procedure in the absence of viral replication (Kolykhalov et al., 1997; Yanagi et al., 1998; Beard et al., 1999). Thus, the appearance of RNA in serum collected subsequently from these tamarins will be strong evidence for the replication competence of the synthetic RNA. Serum will be collected weekly for six weeks, then every other week for an additional 6 weeks from inoculated animals. In addition to RT-PCR for detection of viral RNA, alanine aminotransferase (ALT) levels will be measured as an indicator of liver injury and to assess liver histology in punch biopsies taken at the time of ALT elevation. Maximum viremia and an acute phase ALT response is expected to occur around 14-28 days post-inoculation of infectious RNA (Simons et al., 1995; Schlauder et al., 1995; Karayiannis et al., 1989). Transfections will be considered to have failed to give rise to infectious virus if RNA is not detected in the serum within 12 weeks of inoculation. Successfully infected animals will be followed with twice weekly bleeds until resolution of the viremia, or for 6 months, whichever is longest.

### Example 2

### GBV-B/HCV Chimeras

The GBV-B genome can be used as the acceptor molecule in the construction of chimeric viral RNAs containing sequences of both HCV and GBV-B. Such constructs will allow one to investigate the mechanisms for the different biological properties of these viruses and to discover and investigate potential inhibitors of specific HCV activities (e.g., proteinase) required for HCV replication. Different classes of chimeric viruses are contemplated. These include: (a) replacement of the GBV-B IRES with that of HCV; and (b) replacement of the NS3 major serine proteinase and helicase, and (c) the replacement of the NS5B RNA-dependent RNA polymerase with the homologous proteins of HCV.

The chimeric constructs described in the following sections will be made by PCR mutagenesis, using high fidelity polymerases and oligonucleotide primers designed to include the specific fusions of GBV-B and HCV sequences (Landt et al., 1990). First round PCR reactions will create the desired fusion, and generate a new “primer” to be used in a second PCR reaction spanning the region to a convenient unique restriction site. PCR cycles will be kept to the minimum number necessary for successful amplification, and all segments of viral sequence that are amplified by PCR will be subjected to DNA sequencing to exclude the presence of unwanted PCR-introduced errors. Sequencing will be accomplished at UTMB's core Recombinant DNA Laboratory. Amplified segments will be kept to the minimum by the exchange of cloned cDNA segments spanning convenient restriction sites in subgenomic clones, and where necessary PCR artifacts can be corrected by site-directed mutagenesis (QuickChange mutagenesis kit, Stratagene).

A number of viable positive-strand RNA virus chimeras have been constructed previously in which IRES elements have been swapped between different viruses. Most of these chimeras have involved the exchange of IRES elements between picomaviruses. Others have been successful in constructing viable poliovirus chimeras containing the HCV IRES in place of the native poliovirus IRES (Zhao et al., 1999; Lu and Wimmer, 1996). A similar rhinovirus 14 chimera containing the HCV IRES has been constructed, although its replication phenotype is not as robust as the poliovirus chimera described by Lu and Wimmer (Lu and Wimmer, 1996). More importantly, Frolov et al. (Frolov et al., 1998) recently reported chimeric flaviviruses in which the HCV IRES was inserted into the genetic background of a pestivirus, bovine viral diarrhea virus (BVDV) in lieu of the homologous BVDV sequence. Although these viable chimeric polioviruses and pestiviruses replicate in cell cultures, they are poor surrogates for HCV in animal models as neither virus is hepatotropic or causes liver disease. Importantly, Frolov et al. (Frolov et al., 1998) demonstrated quite convincingly that the requirement for cis-acting replication signals at the 5′ terminus of the pestivirus genome was limited to a short tetranucleotide sequence. This requirement presumably reflects the need for the complement of this sequence at the 3′ end of the negative strand during initiation of positive-strand RNA synthesis. The work of Frolov et al. shows that the IRES of BVDV does not contain necessary replication signals, or that if these are present within the BVDV IRES they can be complemented with similar signals in either the HCV or encephalomyocarditis virus (EMCV, a picomavirus) IRES sequence. Since GBV-B and HCV are more closely related to each other than BVDV and HCV, these studies provide strong support for the viability of chimeras containing the HCV IRES in the background of GBV-B.

Construction of a viable IRES chimera will be enhanced by studies that have documented the sequence requirements and secondary structures of the IRES elements of both HCV and GBV-B (see Lemon and Honda, 1997; Honda et al., 1996; Rijnbrand et al., 1999). To a considerable extent, the work of Frolov et al. (Frolov et al., 1998) was guided by studies of the HCV IRES structure. More recently, these studies have been extended to include a detailed mutational analysis of the GBV-B IRES. The results of these studies indicate that the functional IRES of GBV-B extends from the 5′ end of structural domain II (nt 62) to the initiator AUG codon (nt 446). This segment of the full-length GBV-B clone will be replaced with HCV sequence extending from 5′ end of the analogous domain II within the HCV IRES (nt 42) to the initiator codon at the 5′ end of the HCV open reading frame (nt 341) to construct the candidate chimera, “GB/C:IRES”. The source of HCV cDNA for these studies will be the infectious HCV clone, pCV-H77C, which contains the sequence of the genotype 1a Hutchinson strain virus (Yanagi et al., 1998), whose infectivity in a chimpanzee following intrahepatic inoculation with synthetic RNA transcribed from pCV-H77C has been confirmed.

This GB/C:IRES construct will retain two upstream hairpins within the GBV-B sequence (stem-loops Ia and Ib), and it is thus analogous to the viable “BVDV+HCVde1B2B3H1” chimera of Frolov et al (Frolov et al., 1998). A second chimera can be constructed in which the entire HCV 5′ nontranslated RNA will be inserted in lieu of nts 62-446 of the GBV-B genome (“GB/C:5′NTR”). This construct will add to the inserted HCV sequence the most 5′ stem-loop from HCV (stem-loop I). A similar insertion was shown to substantially increase the replication capacity of BVDV+HCVde1B2B3H1 by Frolov et al. (Frolov et al., 1998), providing a replication phenotype similar to wild-type BVDV in cell culture.

It is important to point out that there is strong evidence from multiple lines of investigation indicating that it will not be necessary to include coding sequence in these IRES chimeras. This is the case even though Reynolds et al. (Reynolds et al., 1995) have argued that the HCV IRES extends past the initiator codon, and into the core-coding region of that virus. Although Lu and Wimmer (Lu and Wimmer, 1996) found it necessary to include HCV core sequence to obtain a viable chimeric poliovirus, the BVDV chimeras reported by Frolov et al. (Frolov et al., 1998) did not contain any HCV coding sequence. This discrepancy may be explained by the observation that the only downstream requirement for full activity of both the GBV-B and HCV IRES elements is the presence of an unstructured RNA segment (Honda et al., 1996; Rijnbrand et al., 1999). Presumably, this facilitates interaction of the viral RNA with the 40S ribosome subunit in the early steps of cap-independent translation (Honda et al., 1996). The 5′ GBV-B coding sequence fulfills this criterion (Rijnbrand et al., 1999).

In vitro Characterization of the Translational Activity of IRES Chimeras.—The fidelity of the genome-length chimeric constructs will be confirmed by sequencing any DNA segments that have been subjected to PCR during the construction process, as well as confirming sequence at the junction sites. In addition, the translational activity of synthetic RNA derived from these constructs will be assessed and compared to the translational activity of the wild-type GBV-B and HCV RNAs. These studies will be carried out in a cell-free translation assay utilizing rabbit reticulocyte lysates (Rijnbrand et al., 1999). Synthetic RNA will be produced by runoff T7 RNA polymerase transcription using as template ClaI-digested plasmid DNA (BamHI digestion in the case of the genome HCV construct) (T7 Megascript kit, Ambion). ³H-UTP will be added to the reaction mix to allow for quantification of the RNA product. Reticulocyte lysates (Promega) will be programmed for translation by the addition of RNA (at least 50% full-length as determined by agarose gel electrophoresis) at 20, 40 and 80 μg/ml, and translation reactions will be supplemented with microsomal membranes (Promega). ³⁵S-Methionine-labelled translation products will be separated by SDS-PAGE, and the quantity of E1 protein produced from each RNA determined by PhosphorImager analysis (Molecular Dynamics). Comparisons of the activity of the HCV IRES in the background of GBV-B and HCV will take into account differences in the methionine content of the E1 proteins of these viruses. Based on previous studies of both the GBV-B and HCV IRES elements (Honda et al., 1996; Rijnbrand et al., 1999), it is expected that these studies will confirm that the HCV IRES will retain nearly full activity when placed within the GBV-B background.

In vivo Characterization of IRES Chimeras. Synthetic RNAs produced from each of the two chimeric GBV-B/HCV constructs (GB/C:IRES and GB/C:5′NTR) will be tested for their ability to induce infection and cause liver disease in susceptible tamarins. These studies will be carried out as described in Example 2. GB/C:5′NTR may generate viremia and liver injury more closely resembling that observed with wild-type GBV-B infection (Frolov et al., 1998).

Chimeric GBV-B/HCV with HCV NS3 in a GBV-B Background. Chimeric flaviviruses containing the HCV NS3 serine proteinase/helicase within the GBV-B background are also contemplated within the present invention. The construction of chimeric flaviviruses containing specific heterologous functional polyprotein domains, however, poses a number of special problems. Unlike the situation with the IRES, where the relevant RNA segments appear to have a unique function restricted to cap-independent translation initiation and interact with host cell macromolecules, viral proteins often have multiple functions and may form specific macromolecular complexes with other viral proteins that are essential for virus replication (Lindenbach and Rice, 1999). Furthermore, such chimeric polyproteins must be amenable to efficient processing by the viral proteinases (NS2/NS3 or NS3). This requires knowledge of the proteinase cleavage specificities as well as specific sites of proteolytic cleavage. Although to date there have been no published studies of the processing of the GBV-B polyprotein, the relatively close relationship between GBV-B and HCV, about 30% overall amino acid identity within the polyprotein (Muerhoff et al., 1995), allows good computer predictions of the alignments of these proteins. The crystallographic structures of both the proteinase and helicase domains of the HCV NS3 protein have been solved (Yao et al., 1997). Thus, both linear alignments and models of the 3D structure of the NS3 proteins of these viruses can provide guidelines for designing specific chimeric fusions that are likely to preserve function.

NS3 Proteinase-Domain Chimeras. In HCV, NS3 contains the major serine proteinase that is responsible for most cleavage events in the processing of the nonstructural proteins, i.e., those that occur at the NS3/4A, 4A/4B, 4B/5A and 5A/5B junctions. The active proteinase domain of HCV is located within the amino terminal third of the NS3 protein (residues 1-181), which shares 31% amino acid identity with the analogous segment of the GBV-B polyprotein (GBV-B vs HCV-BK) (Muerhoff et al., 1995). Importantly, the active site of this proteinase appears to be particularly well conserved in GBV-B. The GBV-B proteinase maintains the residues that are responsible for catalysis and zinc binding in the HCV enzyme (Muerhoff et al., 1995), and unlike the NS3 proteinases of some other flaviviruses preserves the Phe-154 residue that determines in part the S₁ specificity pocket of the enzyme and the preference of the HCV proteinase for substrates with a cysteine residue at the P1 position (Scarselli et al., 1997). Thus, it is not surprising that the relevant proteolytic cleavage sites within the GBV-B polyprotein that are predicted from alignments with the HCV polyprotein all possess a Cys residue at this position. Of greatest significance for the proposed studies, however, is the work of Scarselli et al. (Scarselli et al., 1997) who demonstrated that the GBV-B NS3 proteinase is able to effectively process the polyprotein of HCV in studies carried out in vitro. Using synthetic peptide substrates, these investigators demonstrated that the enzymatic activities of the GBV-B proteinase (residues 1-181) had kinetic parameters similar to the HCV proteinase on NS4A/4B and NS4B/4A substrates HCV. They did not possess reagents allowing a determination of whether the HCV proteinase is able to cleave a GBV-B substrate, but their results indicate that these viral proteinases share important functional properties. Therefore, these similarities suggest that the HCV proteinase could function in lieu of the GBV-B proteinase if used to replace this segment of an infectious GBV-B clone. In addition, studies with sindbis/HCV chimeras have shown that the HCV proteinase can cleave within the framework of a sindbis polyprotein (Filocamo et al., 1997).

In considering the design of these NS3 proteinase chimeras, there are two additional important considerations. First, in HCV, the cleavage between NS2 and NS3 occurs in cis, as the result of a zinc-dependent metalloproteinase that spans the NS2/NS3 junction (Hijikata et al., 1993). As only the NS3 sequences will initially be exchanged, the viability of the resulting chimeras will be dependent upon preservation of the cis-active cleavage across a chimeric NS2/NS3 proteinase domain. The alignment of GBV-B and HCV sequences shows that residues in HCV that have been shown by Grakoui et al., 1993, to be essential for the NS2/NS3 cleavage are conserved in GBV-B (Muerhoff et al., 1995). Additional chimeras that will include the relevant carboxyl-terminal portion of NS2 can also be created.

A second important consideration is that the mature HCV NS3 proteinase functions as a noncovalent assembly of the NS3 proteinase domain and the amino terminal portion of NS4A, a proteinase accessory factor. The details of this association are well known, and have been studied at the crystallographic level (Kim et al., 1996). The N-terminal domain of the folded proteinase contains eight β strands, including one contributed directly by the NS4A peptide backbone. X-ray studies have shown that this array of β strands gives rise to a much more ordered N-terminus. Thus, the presence of the NS4A strand seems likely to contribute to the structure of the substrate-binding pocket. It is not known whether the NS3 proteinase of GBV-B also requires a similar interaction with NS4A of that virus for complete activity, or, if so, whether the NS4A of GBV-B could substitute for NS4A of HCV in forming the fully active NS3 proteinase of HCV. The predicted GBV-B NS4A molecule is 54 amino acid residues in length (Simons, et al., 1995; Muerhoff et al., 1995), just as in HCV. However, the level of amino acid homology between the NS4A molecules is not especially high, and the potential interaction with either NS3 molecule cannot be predicted from this sequence on the basis of available knowledge. To overcome this potential problem, chimeras will be created in which not only the NS3 proteinase domain of GBV-B is replaced, but also the relevant NS4A segment as well, with homologous segments of the HCV polyprotein. The interaction of the HCV NS3 and NS4A domains represents a unique target for antiviral drug design, and it would be beneficial to have this specific interaction present in any virus to be used as a surrogate for HCV in the evaluation of candidate antiviral inhibitors of HCV proteinase in vivo.

The NS3 proteinase chimeras that can be made include “GB/C:NS3P”, which will contain the sequence encoding the first 181 amino acid residues of the HCV NS3 molecule in lieu of that encoding the first 181 residues of GBV-B NS3, and “GB/C:NS314A”, which will include the same NS3 substitution as well as the HCV sequence encoding the amino-terminal segment of NS4A that forms the interaction with NS3. The precise NS4A sequence to be included in the latter chimera will be based on the modeling studies, which may also suggest more effective fusions of the NS3 proteinase domain of HCV with the downstream NS3 helicase domain of GBV-B. The source of HCV cDNA for these studies will be the infectious HCV clone, pCV-H77C, which contains the sequence of the genotype 1a Hutchinson strain virus (Yanagi et al., 1998).

NS3 Helicase Domain Chimeras. In addition to serine proteinase activity located within the amino-third of NS3, the downstream carboxy-terminal two-thirds of the molecule contains an RNA helicase activity. These two functional domains appear to be separated by a flexible spacer, within which the fusion of HCV proteinase or helicase domain sequences with GBV-B sequence will be placed. The exact role of the helicase in the HCV life-cycle is not known, but it is almost certainly required for dsRNA strand-separation during some phase of viral RNA synthesis. The helicase domains of GBV-B and HCV are remarkably well conserved, with some regions within the helicase showing as much as 55% amino acid identity (Muerhoff et al., 1995). The GBV-B helicase is more closely related to the HCV helicase than all other flaviviral NS3 helicases, and it preserves many residues found within the conserved helicase motifs of HCV. Thus the HCV NS3 helicase may be capable of functioning when placed within the polyprotein of GBV-B, and such a chimeric virus may be capable of replication. Residues 182-620 of the GBV-B NS3 molecule will be substituted with the analogous segment of HCV. A chimera will also be made in which the entire NS3 and amino terminal NS4 protein sequences of GBV-B is replaced with the homologous HCV sequences (“GB/C:NS3-4A”). The latter construct will thus represent a dual proteinase-helicase chimera. As with the proteinase chimeras, the HCV cDNA will be derived from pCV-H77C (Yanagi et al., 1998).

In vitro Characterization of NS3 and NS3-NS4A Chimera. Prior to being evaluated for infectivity in susceptible tamarins, RNAs produced in vitro from these clones will characterized in vitro. This evaluation will be restricted to a documentation of the proper processing of the expressed polyprotein (i.e., NS2/NS3 and NS3 proteinase functions), since there are no relevant assays that can determine whether the helicase or RNA-dependent RNA polymerase activities in these polyproteins are sufficient for virus replication. The proteolytic processing of the polyprotein is important, however, as it may be altered either by inclusion of the heterologous HCV NS3 proteinase in lieu of the natural GBV-B protease, or by a change in the folding of the polyprotein induced by inclusion of HCV sequence anywhere within the polyprotein. These studies will be carried out in cell-free coupled transcription/translation assays (“TnT” system, Promega) supplemented with microsomal membranes. Template DNAs will be digested with SalI, which restricts the cDNA within the NS5B coding region. ³⁵S-methionine-labelled translation products will be separated by SDS-PAGE, and the mature NS3 protein identified by its apparent molecular mass. The NS3 and NS5B proteins will be identified by immunoblot analysis using rabbit antisera to the GBV-B NS3 and NS5B proteins. Generation of a mature ˜68 kDa NS3 protein will provide proof of both the cis-active NS2/NS3 cleavage and the NS3-mediated cleavage of NS3/NS4A. Similarly, identification of a mature, processed NS5B molecule will provide further support for the activity of the NS3 proteinase. Controls for these studies will be the wild-type GBV-B polyprotein expressed in similar fashion from the full-length GBV-B clone. If necessary to more clearly demonstrate the processing of the nonstructural proteins in these constructs, subclones representing the nonstructural region of the chimeric sequences could be produced.

In vivo Characterization of NS3 and NS3-4A Chimeras. Synthetic RNAs produced from each of the chimeric GBV-B/HCV constructs described in the preceding section will be tested for their ability to induce infection and cause liver disease in susceptible tamarins. These studies will be carried out using the approach described above.

### Example 3

### Chimeric GBV-B/HCV Containing HCV NS5B in a GBV-B Background

The HCV NS5B molecule contains an RNA-dependent RNA polymerase that plays a central role in replication of the virus. Although this molecule represents a prime target for drug discovery efforts, it has proven difficult to express NS5B in a form that retains enzymatic activity specific for HCV RNA as a substrate. Thus, relatively little is known of the functional activity of the HCV replicase, including structure-function relationships of NS5B. Despite this, the NS5B proteins of GBV-B and HCV appear to be functionally closely related, as they share as much as 43% amino acid identity (Muerhoff et al., 1995). A more important question may be whether an RNA dependent RNA polymerase can act on foreign substrates. However, published work has shown that in vitro purified HCV polymerase has very little specificity for its template, using hepatitis C or globin message with equal fidelity (Behrens et al., 1996; Al et al., 1998). This finding is very similar to that obtained with picomaviral polymerases, where it has been known for many years that in vitro the enzyme exhibits very little specificity. It has always been considered highly likely that this situation would not pertain in vivo where it was thought that the interaction of viral or cellular factors with the 3′ end of the genome would generate template specificity. However, recent reports have shown that the removal of the entire 3′ untranslated sequence (leaving, however, the poly(A) region present) from both the poliovirus and rhinovirus genome does not completely abrogate the infectivity of the virus (Todd et al., 1997). Furthermore, virus, which was recovered after the initial transfections, was shown to have recovered much of the infectivity of the original virus (Todd et al., 1997). The mechanism for this recovery of infectivity is at present unknown, but these results suggest that the HCV polymerase may be able to function to replicate infectious GBV-B/HCV NS5B chimeras.

Thus a chimeric genome-length virus can be created in which the NS5B coding sequence of HCV (amino acids 2422-3014, 593 residues) is inserted within the background of GBV-B in lieu of its native RNA-dependent RNA polymerase (amino acids 2274-2864, 591 residues). This chimeric virus would be valuable for animal studies of candidate antiviral inhibitors of HCV RNA synthesis.

This NS5B chimera would be evaluated to determine that there was proper proteolytic processing of the polyprotein. This would be accomplished by expression of the chimeric polyprotein in a coupled translation-transcription reaction, followed by immunoblot analysis for the mature NS5B protein, as described for the NS3 and NS3-4A chimeras in the preceding section. If these results confirm that the GB/C:5B chimeric polyprotein is processed with release of NS5B, studies in tamarins would progress to determine whether synthetic RNA transcribed from the clone is infectious and capable of causing liver disease in intrahepatically inoculated animals. These studies would be carried out as described above.

A chimeric molecule can be constructed from an infectious GBV-B clone in which the HCV NS3 proteinase or proteinase/helicase sequence would be placed in frame in lieu of the homologous GBV-B sequence, and this chimeric cDNA would be used to generate infectious GBV-B/HCV chimeric viruses by intrahepatic inoculation of synthetic RNA in tamarins. Published studies indicate that the GBV-B and HCV proteinases have closely related substrate recognition and cleavage properties, likely making such chimeras viable and capable of initiating viral replication in appropriate cell types.

### Example 4

### Chimeric Viruses Containing HCV Structural Proteins within a GBV-B Genetic Background, and GBV-B Structural Proteins within an HCV Background

It is well documented that the structural proteins of one flavivirus may in some cases be substituted for those from another member of the family. Such chimeric viruses have been recovered from viruses as distantly related to each other as dengue virus and tick-borne encephalitis virus (Pletnev et al., 1992). More recently, the prM and E proteins of Japanese encephalitis virus have been used to replace the equivalent proteins in a vaccine strain of yellow fever virus to produce a JE/YF chimera (Chambers et al., 1999). These observations suggest that chimeras in which the structural proteins of HCV have been used to replace the homologous proteins of GBV-B may well be viable and capable of replication. The isolation of a chimeric virus containing HCV structural proteins, but having the growth characteristics of GBV-B virus, could answer many fundamental questions concerning the structure and interaction of these proteins in HCV. They would also be useful in addressing the nature of the immune response to HCV structural proteins in infected primates (Farci et al., 1992). More to the point of this application, the availability of such chimeric viruses would allow studies of candidate HCV vaccines to be carried out in the tamarin model. This would be a major advance, because at present such studies are limited to chimpanzees (Choo et al., 1994).

The basis for the difference in the host ranges of HCV and GBV-B is completely unknown. Among many other possibilities, it is conceivable that the host range is dependent upon the availability of a specific receptor(s). If this were the case, host range might be dependent upon the envelope proteins that must interact with the putative cellular receptor. Thus, a chimeric virus containing the envelope proteins of HCV within the genetic background of GBV-B might be noninfectious in tamarins (but potentially infectious in chimpanzees). Thus, a finding that both structural protein chimeras are noninfectious in the tamarin, may require the construction of complementary chimeras in which the relevant GBV-B structural proteins will be inserted into the background of an infectious HCV clone. If inclusion of the GBV-B envelope proteins within the backbone of HCV confers on the resulting chimera the ability to replicate in tamarins, it will confirm an important role for the structural proteins in defining the different host ranges of these viruses. More importantly, the resulting virus would be an exceptionally valuable resource for future studies as it would contain all of the nonstructural replication elements, as well as the 5′ and 3′ nontranslated regions, of HCV. Such a virus would allow the tamarin model to be used to address many unresolved issues in HCV biology and pathogenesis.

Construction and Evaluation of Structural Protein Chimeras. In designing structural protein chimeras, it is important to note that the two envelope proteins of HCV, E1 and E2, form noncovalent heterodimeric complexes that are likely to be important in the assembly of infectious virus particles. This is not known to be the case with the envelope proteins of GBV-B, but it is likely given similarities in the sizes and hydropathy profiles of these proteins (Simons et al., 1995; Muerhoff et al., 1995). Accordingly, the E1 and E2 proteins will be replaced as a unit, and chimeras containing only one of these proteins from the heterologous virus will generally not be produced. First, a chimera will be created where the E1 and E2 regions of GBV-B virus are replaced with those of HCV, “GB/C:E1-2”. The source of HCV cDNA for these constructions will be pCV-H77C (Yanagi et al., 1998). A chimera will also be made in which the core protein, in addition to the envelope proteins, is replaced with the homologous proteins of HCV (“GB/C:Co-E2”). Additional chimeras will be made to determine whether tamarins can be infected with chimeras containing the GBV-B structural proteins within the genetic background of HCV. These will include “C/GB:E1-2” and “C/GB:Co-E2”. The backbone for these chimeras will be pCV-H77C, the infectious genotype 1a cDNA clone developed in the Purcell laboratory at NIAID (Yanagi et al., 1998).

The specific amino acid sequences of GBV-B to be replaced with the homologous segments of HCV have been determined by alignments of the GBV-B and HCV sequences, coupled with the location of signalase cleavage sites predicted to be present within the amino terminal third of the GBV-B polyprotein using the computer algorithm of Von Heijne. These predicted signalase cleavages lie between residues 156/157 (core/E1), aa 348-349 (E1/E2) and 732/733 (E2/NS2) in the GBV-B sequence. Thus, the chimera GB/C:E1-2 will contain sequence encoding HCV aa 192-809 in lieu of that encoding aa 157-732 in GBV-B, while the insertion in the GB/C:Co-E2 chimera will extend from the initiator AUG codon (aa 1) to residue 809 in HCV, and will be spliced into GBV-B in lieu of the segment encoding aa 1-732 in the GBV-B clone. The complementary chimeras to be constructed within the background of HCV will involve exchanges of the same segments of the genomes.

### Methods

Infectious, genomic-length cDNAs of GBV-B (Martin et al., 2003) and the H77 isolate of HCV genotype 1a (obtained from Dr. R. H. Purcell, N.I.H., USA, under MTA, Yanagi et al., 1997), both cloned downstream of the T7 RNA polymerase promoter, were used as backbones to construct chimeric cDNAs.

To carry out exact substitutions of GBV-B sequences by analogous HCV sequences or vice-versa, a PCR-based fusion strategy was used, involving the synthesis of 3 PCR fragments each with 27-29 nucleotide (nt)-overlaps. For example, a central PCR fragment corresponding to the GBV-B sequence to be inserted, as well as 2 PCR fragments corresponding to HCV sequences framing the sequence to substitute, each containing a unique restriction site for cloning purposes, were synthetized from appropriate cDNA templates. A final chimeric PCR fragment was generated using a mixture of the 3 fragments with overlaps. After digestion at the unique sites at the 5′ and 3′ ends, the chimeric fragment was cloned between the same sites in the GBV-B parental cDNA.

pHC/C-p13^{GB} et pHC/E1-p13^{GB-}—Restriction enzymes AgeI (nt 155) and BstZ17I (nt 3643) of pCV-H77C (Yanagi et al., 1997) within the HCV cDNA were used to insert chimeric HCV/GBV-B/HCV fragments that included sequences coding for E1-E2-p13 (nts 914-2614) or C-E1-E2-p13 (nts 446-2614) of GBV-B, generating plasmids pHC/E1-p13^{GB} and pHC/C-p13^{GB}, respectively (FIG. 1).

pGB/C-p7^{HC} et pGB/E1-p7^{HC}—Restriction enzymes SspI (nt 11613, in the vector sequences upstream of the T7 promoter) and AflIII (nt 3414, in the GBV-B cDNA) of pGBV-B/2 (Martin et al., 2003) were used to insert chimeric GBV-B/HCV/GBV-B fragments that included sequences coding for E1-E2-p7 (nts 915-2768) or C-E1-E2-p7 (nts 342-2768) of HCV, generating plasmids pGB/E1-p7^{HC} and pGB/C-p7^{HC}, respectively (FIG. 1).

pHC/C-NS3ₚᵣₒ^{GB}-Ubi et pHC/E1-NS3ₚᵣₒ^{GB}-Ubi—Chimeric PCR fragments spanning nts 1714-3835 of the GBV-B cDNA, followed by the sequence of the ubiquitin gene and nts 3420-3784 of the HCV cDNA were generated by a PCR-based fusion strategy. The cDNA fragments between restriction sites PmeI and SgrAI, in the GBV-B E2 sequence and in the HCV NS3 sequence, respectively, of plasmids pHC/C-p13 GB and pHC/E1-p13 GB were replaced by the newly synthetized PCR fragment encoding (E2Δ-NS2 NS3ₚᵣₒ)^{GB}-Ubi-(NS3Δ)^{HC} (Δ denoting a truncated form of the protein), generating plasmids pHC/C-NS3ₚᵣₒ^{GB}-Ubi and pHC/E1-NS3ₚᵣₒ^{GB}-Ubi, respectively (FIG. 2).

pGB/C-NS3ₚᵣₒ^{HC}-Ubi et pGB/E1-NS3ₚᵣₒ^{HC}-Ubi—Chimeric PCR fragments spanning nts 2225-3986 of the HCV cDNA, followed by the sequence of the ubiquitin gene and nts 3266-4678 of the GBV-B cDNA were generated by a PCR-based fusion strategy. The cDNA fragments between restriction sites SacI and BsaBI, in the HCV E2 sequence and in the GBV-B NS3 sequence, respectively, of plasmids pHC/C-p13^{GB} and pHC/E1-p13^{GB} were replaced by the newly synthetized PCR fragment encoding (E2Δ-NS2 NS3ₚᵣₒ)^{HC}-Ubi-(NS3Δ)^{GB} (Δ denoting a truncated from of the protein), generating plasmids pGB/C-NS3ₚᵣₒ^{HC}-Ubi and pGB/E1-NS3ₚᵣₒ^{HC}-Ubi, respectively (FIG. 2).

In all chimeric cDNAs generated in the backbone of the GBV-B cDNA, the 3 XhoI sites present in HCV C and E1 sequences were eliminated, so that the XhoI site located downstream of the 3′ end of the cDNA remains unique in the corresponding chimeric plasmids and could be used to linearize cDNAs prior to in vitro transcription. Similarly, in the cDNAs generated in the HCV backbone, the XbaI site was destroyed in the GBV-B E2 sequence. This was carried out by a PCR-based fusion strategy that introduced silent changes within the existing restriction sites.

### Results

Analysis of the translational competence of chimeric genomes.—For both HCV and GBV-B, IRES-driven polyprotein translation initiation is modulated by the nature and probably the structure of the sequence present downstream of the initiating AUG (Rijnbrand et al., 2001).

The inventors therefore determined whether the fusion of heterologous 5′NTR and core sequences in the case of chimeric genomes GB/E1-p7^{HC}, HC/E1-p13^{GB}, GB/E1-NS3ₚᵣₒ^{HC}-Ubi, and HC/E1-NS3ₚᵣₒ^{GB}-Ubi could alter the translational competence of these genomes.

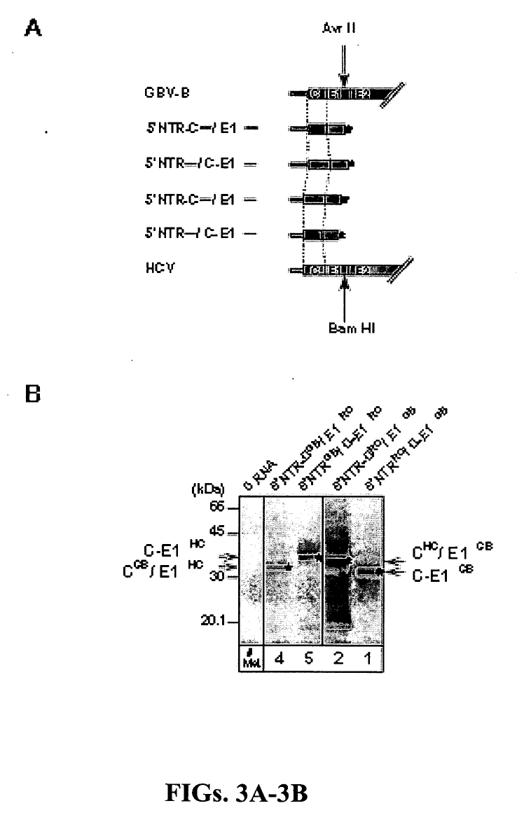

To study this, the inventors used an in vitro translation system in rabbit reticulocyte lysates programmed with RNAs transcribed from truncated cDNA templates that had been linearized within the E1 coding sequence, either at the AvrII restriction site (in GBV-B) or the BamHI site (in HCV) (FIG. 3A)]. Hence, a unique, short polypeptide was synthetized in the absence of canine microsomal membranes in each case. The efficiencies of translation directed from IRESes present in GB/C-p7^{HC} and GB/E1-p7^{HC} were compared, the latter containing an IRES similar to parental GBV-B IRES, and from IRESes present in HC/C-p13^{GB} and HC/E1-p13^{GB}, that contains an IRES similar to that of parental HCV.

Serial dilutions of in vitro transcribed RNA were quantified precisely on agarose gels and 10 ng/μl of RNA were used to program in vitro translation reactions in the presence of [³⁵S]Met and in the absence of microsomal membranes. The translation of chimeric 5′NTR-C^{GB}/E1Δ^{HC} RNA (FIG. 3) generated a 34 kDa product, that corresponds to C^{GB} fused to 148 amino acids of E1^{HC}, whereas the translation of chimeric 5′NTR^{GB}/C-E1Δ^{HC} RNA produced a 37 kDa polypeptide corresponding to C^{HC} fused to the same 148 residues of E1^{HC} (FIG. 3B). Comparative quantitations of the resulting products were carried out by densitometry after analysis of the gel with a PhosphorImager (Molecular Dynamics) and corrected with respect to the number of methionine residues present in each polypeptide.

The inventors found that all constructs were translation-competent. However, the translational efficiency of 5′NTR^{GB}/C-E1Δ^{HC} RNA, that contains heterologous 5′NTR and core sequences, was decreased by 55% with respect to that of 5′NTR-C^{GB}/E1Δ^{HC} RNA. Similarly, the translational efficiency of 5′NTR^{HC}/C-E1Δ^{GB} RNA was decreased by 45% with respect ot that of the 5′NTR-C^{HC}/E1Δ^{GB} (FIG. 3B). Therefore, both parental GBV-B and HCV IRESes followed by homologous core sequences are more efficient in directing translation than a chimeric IRES composed of GBV-B 5′NTR sequences followed by HCV core sequences, or an HCV IRES followed by GBV-B core sequences. Although it is not impossible that a two-fold decrease in the translational capacities of chimeric genomes that contain heterologous 5′NTR and core sequences may affect the overall viral production, the data essentially show that all chimeric genomes are translationally competent.

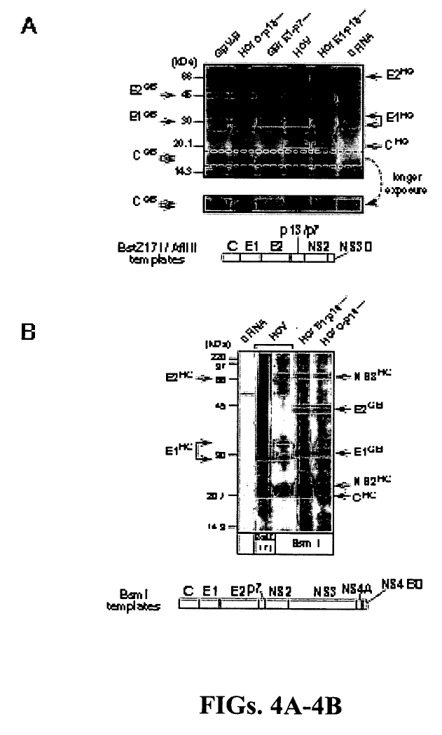

Analysis of the processing at heterologous junctions within the chimeric polyproteins.—[0191] To monitor proteolytic processing of chimeric GBV-B/HCV structural precursors by cellular signalases, in vitro translation reactions in rabbit reticulocyte lysates were programmed with subgenomic RNAs in the presence of either ³⁵S-methionine (FIG. 4B) or ³⁵S_cysteine (FIG. 4A), as well as canine pancreatic microsomal membranes. Since the predicted GBV-B core protein lacks methionine residues with the exception of the initiating methionine residue, ³⁵S-cysteine was utilized to visualize this polypeptide.

Parental or chimeric cDNA templates in the GBV-B or HCV backbones were linearized with restriction enzymes AflIII or BstZ17I, respectively. Such linearized cDNA templates were transcribed in vitro using T7 RNA polymerase, generating subgenomic RNAs with a coding capacity corresponding to C-E1-E2-NS2 followed by a short N-terminal segment of NS3 (FIG. 4A). Processing of chimeric precursors HC/E1-p13^{GB} and HC/C-p13^{GB} generated two polypeptides with respective electrophoretic mobilities indistinguishable from those of parental GBV-B E1 and E2 proteins. In addition to envelope glycoproteins, cleavage of HC/E1-p13^{GB} precursor also yielded a polypeptide that co-migraged with the core protein originating from parental HCV precursor (CHC) by SDS-PAGE, exhibiting a ˜21 kDa apparent molecular weight (FIG. 4A). This is consistent with what has been described in the literature for the mature HCV core protein (Yasui et al., 1998; McLauchlan et al., 2002). In addition, cleavage of HC/C-p13^{GB} generated a pair of polypeptides that co-migrated with polypeptides presumably corresponding to an immature form of GBV-B core protein containing the E1 signal peptide and the mature form (FIG. 4A).

Conversely, processing of chimeric precursors containing HCV structural proteins in the GBV-B backbone (GB/E1-p7^{HC}, GB/C-p7^{HC}) proved to generate polypeptides with expected electrophoretic mobilities, identical to those of parental proteins with respect to HCV E1 and E2 glycoproteins and GBV-B core protein (GB/E1-p7^{HC}, (FIG. 4A)). It should be stressed that, whether derived from parental HCV or chimeric GBV-B/HCV precursors, HCV glycoprotein E1 is present in two distinct forms that are likely to reflect various degrees of glycosylation. The reason why HCV core protein was observed only in its mature form, while GBV-B analogous protein seemed to be present in what the inventors anticipate to represent both immature and mature forms is unclear at present, but might reflect more efficient protein maturation in this system in the case of HCV. Overall, this study strongly suggests that there is no defect in cleavages at heterologous C/E1 or homologous E1/E2 junctions in the chimeric substrates.

To analyze processing at the heterologous junction engineered between p13 and NS2 in chimeric polyproteins within the HCV backbone, RNA templates were used that can encode a precursor comprising C-NS3 sequences followed by an N-terminal part of NS4B. For that matter, cDNAs were linearized with BsmI prior to RNA transcription (FIG. 4B). In vitro translation reactions carried out in the presence of microsomal membranes showed that chimeric precursors HC/E1-p13^{GB} and HC/C-p13^{GB} released GBV-B E1 and E2 proteins, as well as HCV core protein in the case of chimera HC/E1-p13^{GB}, as expected from results above. In addition, a polypeptide of approximately 23 kDa, similar to that derived from the processing of HCV native precursor was observed (FIG. 4B). This 23 kDa-polypeptide was identified as HCV NS2 protein on the basis of its absence in the pattern of translated products generated from an HCV precursor that does not include an intact NS3 proteinase domain, as required for the NS2/NS3 cleavage to occur (FIG. 4B). The release of NS2HC is further substantiated by the concomittent production of a polypeptide with an apparent molecular weight of ˜67 kDa, that was identified as HCV NS3 protein (FIG. 4B) by immunoprecipitation with relevant antibodies.

These studies with HCV chimeras containing GBV-B structural proteins strongly suggest that there is accurate processing at the heterologous p13GB/NS2HC junction. Similarly, although GBV-B NS2 was not observed in such in vitro translation patterns, the converse p7HC/NS2GB junction appeared to be accurately processed in chimeras constructed in the GBV-B backbone since polypeptides analogous to HCV E2 and GBV-B NS3 proteins were released from these chimeric precursors.

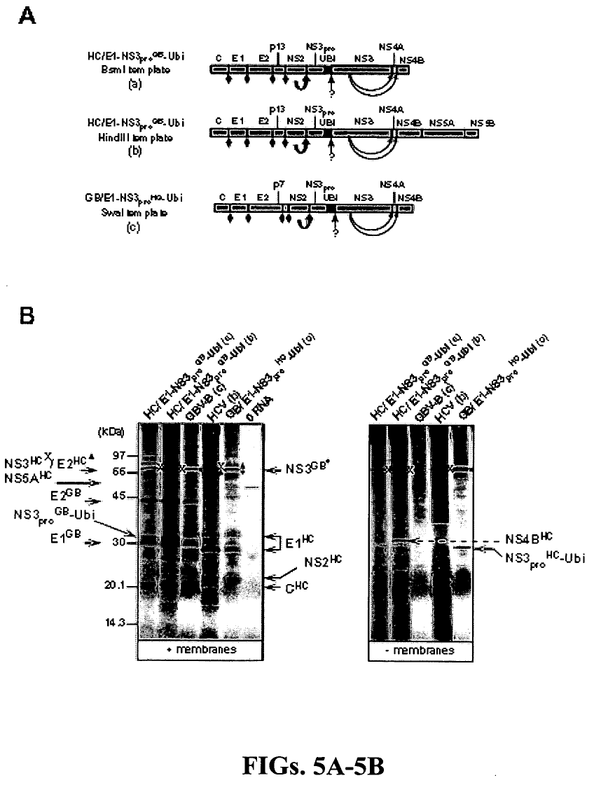

Similarly, a detailed study of the proteolytic processing of the chimeric polyproteins expressed from RNAs GB/C-NS3ₚᵣₒ^{HC}-Ubi, GB/E1-NS3ₚᵣₒ^{HC}-Ubi, HC/C-NS3ₚᵣₒ^{GB}-Ubi, and HC/E1-NS3ₚᵣₒ^{GB}-Ubi has been carried out (for an exmaple, see FIG. 5). In particular, these studies demonstrated that there is efficient cleavage at the engineered NS2/NS3pro and Ubi/NS3 junctions.

Analysis of the replication capacity of the chimeric genomes.—In the absence of reverse genetic systems in cell culture for either HCV or GBV-B, the inventors were studied the replication competence of chimeric RNAs generated in the HCV backbone (HC/E1-p13GB et HC/C-p13^{GB}). Sequences coding for structural proteins (C, E1, E2), as well as those coding for p7 and NS2 do not appear to be involved in RNA replication, since a subgenomic HCV RNA devoid of all these sequences does replicate in Huh-7 cells. The inventors thus hypothetized that the substitution of HCV sequences coding for (C)-E1-E2-p7-(NS2) by analogous sequences from GBV-B would not dramatically impair the replication of chimeric HCV/GBV-B genomes. However, the inventors analyzed the impact of decreased translational efficiency of some genomes, or potential impairment of polyprotein cleavage kinetics on genome replication.

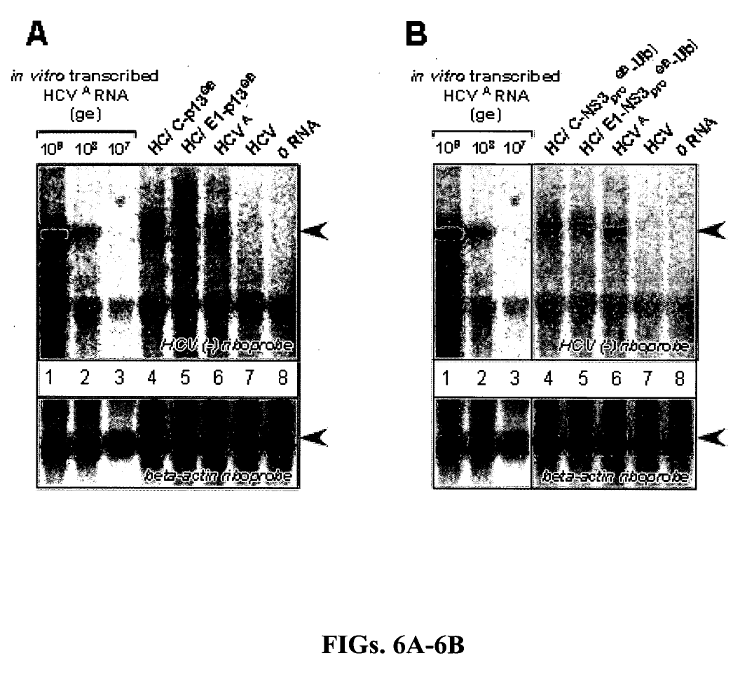

Replication-competent, bicistronic RNAs in Huh-7 cells have essentially been described in the context of HCV genotype 1b (Lohmann et al., 1999). However, Yi et al. recently demonstrated that a monicistronic RNA derived from the H77 strain of HCV genotype 1a (H77c/QR/VI/KR/KR^{5A}/SI) was capable of replication in Huh-7 cells (Yi and Lemon, 2004). This RNA (whcih is referred to as “HCV^{A}” for adapted HCV) contains 5 coding mutations within NS3, NS4A and NS5A coding sequences with respect to the corresponding infectious RNA (Yanagi et al., 1997), that confer to HCV^{A} a robust replication phenotype in cell culture. Four days after transfection of Huh-7 cells with RNA transcripts derived from HCV^{A}, approximately 3×10⁷ genome equivalents per μg of total cellular RNA were detected, whereas RNA from the non-adapted HCV molecular clone remained undetectable (FIG. 6).

In order to study the replication capacity of chimeric genomes generated in the HCV backbone, chimeric GBV-B/HCV sequences from HC/E1-p13^{GB} and HC/C-p13^{GB} constructs were transferred into the backbone of the adapted HCV genome (HCV^{A}), thus generating corresponding chimeric HCA/GBV-B cDNAs. Synthetic RNAs were transcribed in vitro from HC^{A}/E1-p13^{GB}, HC^{A}/C-p13^{GB}, HCV^{A}, and non-adapted HCV cDNAs that have been linearized at the XbaI site and 5 μg of these RNAs used to transfect 2×10⁶ Huh-7 cells. At 4 days post-transfection, total cellular RNAs were prepared and 7.5 μg of RNA, as measured by optical density, were loaded on a denaturing agarose gel and analyzed by Northern blot with an [α-³²P]-UTP riboprobe of negative polarity specific for the 3′ end of the HCV genome. Housekeeping β-actin mRNA was also monitored in each sample in order to normalize quantitations of viral RNAs with respect to fixed amounts of cellular RNA.

As shown in FIG. 6A, chimeric HCA/E1-p13^{GB} RNA replicated twice as efficiently than chimeric HC^{A}/C-p13^{GB} which encodes an heterologous core protein, but both RNAs replicate as robustly, if not better, than parental HCV^{A} RNA. The RNA detected does not reflect residual input RNAs but are clearly derived from de novo synthesis since no RNA was detected after transfection of non-adapted HCV RNAs.

These results show that the substitution of HCV sequences coding for C-E1-E2-p7 or E1-E2-p7 by GBV-B analogous sequences does not alter the RNA replication capacity. However, it is interesting to note that heterologous C and 5′NTR sequences result in a 50% decrease in replication efficiency, when compared to homologous C and 5′NTR sequences (HCA/C-p13GB versus HCA/E1-p13GB).

The replication capacity of HC/E1-NS3ₚᵣₒ^{GB}-Ubi and HC/C-NS3ₚᵣₒ^{GB}-Ubi chimeric RNAs were analyzed in cell culture after transferring chimeric sequences into the backbone of HCV^{A}. Northern blots revealed that the HC^{A}/E1-NS3ₚᵣₒ^{GB}-Ubi chimeric RNA replicated as efficiently as the parental HCV^{A} genome, whereas the HC^{A}/C-NS3ₚᵣₒ^{GB}-Ubi chimeric genome replicated at approximately 50-60% of the parental genome (FIG. 6B). These results demonstrate that these two other chimeric genomes are also replication-competent, although the fusion of an heterolgous core sequence to 5′NTR resulted in a decrease in replication efficiency.

Altogether, these data suggest that the co-substitution of the core coding sequence with those of envelope proteins is somewhat detrimental to RNA replication, whether the substitution involves NS2 sequence or not. This could be the result of a decreased translational capacity of these chimeric RNAs. In addition, the co-substitution of NS2 sequence and/or the insertion of the ubiquitin sequence also resulted in a two-fold decrease in genome replication capacity.

In the absence of any available model system in the laboratory to study GBV-B RNA replication, the replication capacity of the chimeras constructed in the GBV-B backbone could not be assessed.

Assembly of proteins derived from chimeric structural precursors expressing heterologous capsid and envelope proteins into virus-like particles.—In order to investigate whether heterologous capsid and envelope proteins of GBV-B and HCV could assemble to form viral particles (question relevant to chimeras GB/E1-p7^{HC}, HC/E1-p13^{GB}, GB/E1-NS3ₚᵣₒ^{HC}-Ubi, HC/E1-NS3ₚᵣₒ^{GB}-Ubi), an expression system based on recombinant baculoviruses was used that allowed the production of virus-like particles, as initially described for HCV by Baumert et al. (1998). For that purpose, cDNA sequences encoding chimeric GBV-B/HCV structural precursors corresponding to either GBV-B core protein followed by HCV E1-E2-p7 or, conversely, HCV core protein followed by GBV-B E1-E2-p13 were cloned into transfer plasmid pVL1392 (Pharmingen). Correspondingly, parental GBV-B and HCV cDNA sequences encoding C-p13 or C-p7 precursors, respectively, were cloned in pVL1392 dowstream of the baculovirus DNA polyhedrin promoter. After homologous recombination between the recombinant pVL1392 DNAs and baculovirus DNA in Sf9 cells, two parental GBV-B and HCV recombinant baculoviruses, Bac-C-p13^{GB} and Bac-C-p7^{HC}, as well as two chimeric GBV-B/HCV recombinant baculoviruses, Bac-C^{GB}/E1-p7^{HC} and Bac-C^{HC}/E1-p13^{GB} were thus obtained.

The inventors sought virus-like particle (VLP) formation in Sf9 cells infected with recombinant baculoviruses expressing either chimeric GBV-B/HCV structural precursors or parental, HCV or GBV-B precursors. Since nothing was known about the capability of a C-p13 precursor of GBV-B, such as the one expressed here via a recombinant baculovirus, to drive assembly of VLPs either in insect or in mammalian cells, the inventors first focused on a comparative analysis of C^{GB}/E1-p7^{HC} and HCV precursors.

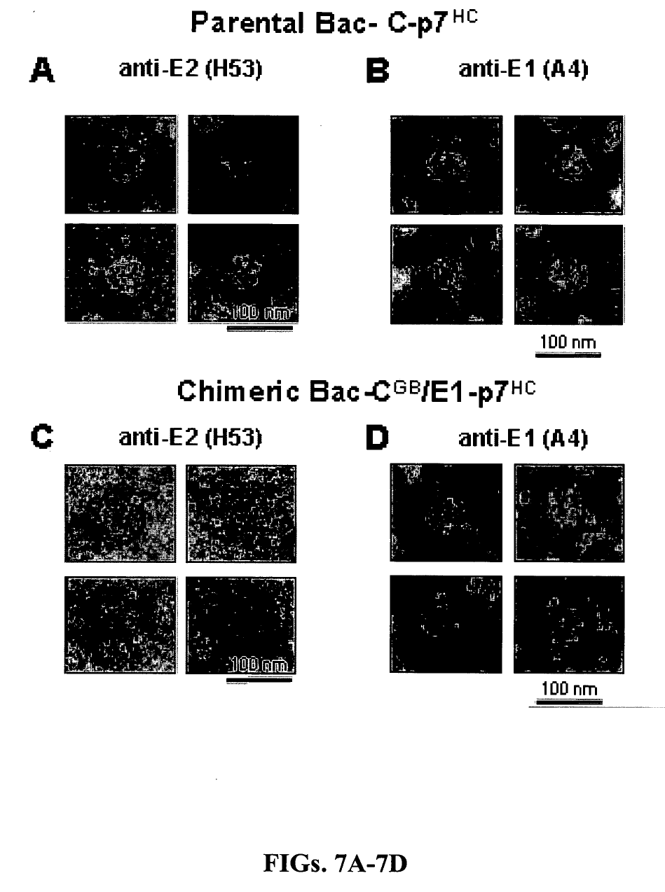

Cytoplasmic extracts prepared at 3 days after infection of Sf9 cells with parental Bac-C-p7^{HC} were concentrated through a 30% sucrose cushion, then fractionated on a 20-60% sucrose gradient. The polypeptide content of each fraction of the sucrose gradients was examined by immunoblotting with a mixture of monoclonal antibodies specific for HCV C, E1 or E2. Fractions 12-13 contained all three structural polypeptides, core and both glycoproteins, indicative of the likelihood to find assembled VLPs in these fractions. To further demonstrate the presence of VLPs in fractions 12-13, a pool of these fractions was concentrated and dialyzed for sucrose elimination prior to analysis by electron microscopy. After negative staining of such preparations, spherical structures approximately 55 nm in diameter were observed. Further immunogold labeling with either monoclonal antibody A4 directed to HCV E1, or monoclonal antibody H53 that recognizes HCV E2 in a conformation-dependent fashion, was performed. Most spherical structures were heavily and specifically marked by gold grains with both A4 and H53 as primary antibodies, but not with monoclonal antibodies specific for HCV core, strongly suggesting that they represent VLPs with both glycoproteins at their surface (FIGS. 7A-7B).

Similar analyses were performed with chimeric virus Bac-CGB/E1-p7^{HC}-infected cell extracts. Immunoblot probing of each sucrose fraction with a mixture of anti-HCV E1 and E2 antibodies showed that the vast majority of E1 and E2 envelope proteins were present in the same fractions (#12-13) as those containing HCV VLPs from Bac-C-p7^{HC}-infected cells. Furthermore, immunoblotting with anti-GBV-B core polyclonal antibodies revealed that GBV-B core co-localized with HCV envelope proteins in the same fractions. Immune electron microscopy work demonstrated particles of similar shape and sizes (61 nm in diameter) by negative staining as VLPs derived from the HCV structural precursor. Most chimeric particles, with the exception of those with a smooth surface that looked like empty structures were covered with gold grains upon immunogold labeling with A4 and H53 antibodies (FIGS. 7C-7D).

The facts that the shape and sizes of chimeric GBV-B/HCV VLPs are close to those of HCV VLPs produced in an analogous system, that these chimeric VLPs exhibit both HCV envelope proteins at their surface, and that GBV-B core protein co-localized in the relevant fractions, are all supportive of the capability of GBV-B core protein to assemble with HCV envelope proteins and form VLPs.

Analysis of the infectivity of genome-length, chimeric RNAs in tamarins.—The promising results obtained in translation, processing, replication and assembly studies of the structural chimeras prompted the assay of the infectivity of full-length chimeric genomes in tamarins.

RNAs were transcribed in vitro from chimeric cDNAs that had previously been linearized at the unique XhoI or XbaI sites downstream of the 3′end of the viral cDNAs in GBV-B or HCV backbones, respectively. Chimeric GB/E1-p7 and GB/C-p7 Hc RNAs were then inoculated separately into the liver of a single GBV-B naïve tamarin (S. oedipus), while a mixture of chimeric HC/E1-p13^{GB} and HC/C-p13 RNAs was inoculated to another single animal. A mixture of the four chimeric RNAs, GB/E1-NS3ₚᵣₒ^{HC}-Ubi, GB/C-NS3ₚᵣₒ^{HC}-Ubi, HC/E1-NS3ₚᵣₒ^{GB}-Ubi, HC/C-NS3ₚᵣₒ^{GB}-Ubi was inoculated to two animals (S. mystax).

Viral replication was monitored by periodic testing of tamarin sera for genomic RNA using appropriate GBV-B (primers and probe in the NS5A coding region) or HCV (primers ans probe in the 5′NTR) real-time, quantitative RT-PCR assays. Onset of hepatitis was followed by testing for serum transaminase (ALT) levels.

In contrast to viral replication that developed over weeks 1 to 10-20 after inoculation of GBV-B wild-type RNA (Martin et al., 2003), no sign of viral replication was detected in animals inoculated with either GB/E1-p7^{HC}, GB/C-p7^{HC}, GB/E1-NS3ₚᵣₒ^{HC}-Ubi+GB/C-NS3ₚᵣₒ^{HC}-Ubi, nor with HC/E1-p13 or HC/C-p13^{GB}.

In one animal out of the two animals inoculated with the 4 genomes containing substitutions extending into the NS2 sequence, a low-level replication (100-1000 genome equivalents/ml) of either chimera HC/E1-NS3proGB-Ubi or HC/C-NS3proGB-Ubi was reproducibly detected at weeks two and four post-inoculation, but no robust replication was further detected. This is reminiscent of what has been observed with the GBV-B IRES chimera, which inoculation resulted in such a low-level replication at early time-points, followed by undetectable replication and a sudden raise in virus replication starting at week 12 post-inoculation. This reflected the requirement for adaptive mutations that arose in one animal out of two inoculated with this chimeric RNA.

In order to check upon such a requirement for adaptive changes in the genome of the structural chimeras, other animals will be inoculated with chimeras HC/E1-NS3proGB-Ubi and HC/C-NS3proGB-Ubi, individually or mixed, but in the absence of other chimeras in the backbone of GBV-B.

### Example 5

### Further Characterization of Rescued Chimeric Viruses

Where infection with chimeric viruses is induced in animals that are injected within the liver with synthetic RNA, this virus will be passaged in GBV-B naïve tamarins to further characterize the nature of the infection induced by the chimera. This will be accomplished by taking a pool of the 3 highest titer GBV-B RNA-containing serum specimens from the animal that was successfully transfected with RNA, and inoculating 1 mL of a 1:100 dilution of this pool intravenously into two susceptible animals. These animals will be monitored for infection and liver disease. These animals will be followed until resolution of the viremia and appearance of antibodies detectable in immunoblots with GST-NS3 protein expressed in E. coli, or for at least 6 months should an animal sustain a chronic infection. RT-PCR amplification of chimeric segments of the genome may be employed to determine whether the altered phenotype results from mutations within the heterologous portion of the genome.

### Example 6

### Use of GBV-B as Model for HCV

GBV-B and/or GBV-B/HCV chimeras can be used as a model for HCV. Such studies will allow one to investigate the mechanisms for the different biological properties of these viruses and to discover and investigate potential inhibitors of specific HCV activities (e.g., proteinase) required for HCV replication. GBV-B/HCV viruses may be used in preclinical testing of candidate HCV NS3 proteinase inhibitors or other inhibitors of HCV.

Candidate Substances—As used herein the term “candidate substance” refers to any molecule that is capable of modulating HCV NS3 proteinase activity or any other activity related to HCV infection. The candidate substance may be a protein or fragment thereof, a small molecule inhibitor, or even a nucleic acid molecule. It may prove to be the case that the most useful pharmacological compounds for identification through application of the screening assay will be compounds that are structurally related to other known modulators of HCV NS3 proteinase activity. The active compounds may include fragments or parts of naturally-occurring compounds or may be only found as active combinations of known compounds that are otherwise inactive. However, prior to testing of such compounds in humans or animal models, it will be necessary to test a variety of candidates to determine which ones have potential.

Accordingly, the active compounds may include fragments or parts of naturally-occurring compounds or may be found as active combinations of known compounds that are otherwise inactive. As such, the present invention provides screening assays to identify agents that are capable of inhibiting proteinase activity in a cell infected with chimeric GBV-B/HCV viruses containing the HCV proteinase. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other compounds that may be designed through rational drug design starting from known inhibitors of proteinases or from structural studies of the HCV proteinase.

The candidate screening assays are simple to set up and perform. Thus, in assaying for a candidate substance, after obtaining a chimeric GBV-B/HCV virus with infectious properties, a candidate substance can be incubated with cells infected with the virus, under conditions that would allow measurable changes in infection by the virus to occur. In this fashion, one can measure the ability of the candidate substance to prevent or inhibit viral replication, in relationship to the replication ability of the virus in the absence of the candidate substance. In this fashion, the ability of the candidate inhibitory substance to reduce, abolish, or otherwise diminish viral infection may be determined.

“Effective amounts” in certain circumstances are those amounts effective to reproducibly reduce infection by the virus in comparison to the normal infection level. Compounds that achieve significant appropriate changes in activity will be used. Candidate compounds can be administered by any of a wide variety of routes, such as intravenously, intraperitoneally, intramuscularly, orally, or any other route typically employed.

It will, of course, be understood that all the screening methods of the present invention are useful in themselves notwithstanding the fact that effective candidates may not be found. The invention provides methods of screening for such candidates, not solely methods of finding them.

In vitro Assays—In one particular embodiment, the invention encompasses in vitro screening of candidate substances. Using a cell line that can propagate GBV-B in culture, in vitro screening can be used such that GBV-B or HCV virus production or some indicator of viremia is monitored in the presence of candidate compounds. A comparison between the absence and presence of the candidate can identify compounds with possible preventative and therapeutic value.

In Vivo Assays—The present invention also encompasses the use of various animal models to test for the ability of candidate substances to inhibit infection by HCV. This form of testing may be done in tamarins.

The assays previously described could be extended to whole animal studies in which the chimeric virus could be used to infect a GBV-B permissive primate, such as a tamarin. One would then look for suppression of viral replication in the animal, and a possible impact on liver disease related to replication of the infectious chimeric virus. The advantage of this in vivo assay over present available assays utilizing HCV infection in chimpanzees is the reduced cost and greater availability of GBV-B permissive nonhuman primate species.

Treatment of animals with test compounds will involve the administration of the compound, in an appropriate form, to the animal. Administration will be by any route that could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, intraperitoneal injection, and oral administration.

Determining the effectiveness of a compound in vivo may involve a variety of different criteria. Such criteria include, but are not limited to, survival, reduction of rate of infection, arrest or slowing of infection, elimination of infection, increased activity level, improvement in liver function, and improved food intake.

### Example 7

### Use of Infectious GBV-B/HCV Chimeras as Vaccines

Infectious GBV-B/HCV chimeras expressing HCV envelope proteins will have utility as a vaccine immunogen for hepatitis C. Such clones clearly have the potential to be constructed as chimeras including relevant hepatitis C virus sequences in lieu of the homologous GBV-B sequence, providing unique tools for drug discovery efforts.

Chimeric viruses containing the envelope proteins of hepatitis C virus (as described in the attached) would confer the antigenic characteristics of hepatitis C virus on the chimera. These chimeras may have the ability to replicate in chimpanzees (and thus humans) by virtue of the fact that the chimeric envelope is now able to interact with the human hepatocyte cell surface, a necessary first step in virus replication. Therefore, the chimeric virus, while able to infect and replicate in humans, may not cause much or any disease—the reasoning here is that the genetic backbone of the chimera that encodes the nonstructural proteins of GBV-B has not evolved for replication in human cells and thus may not replicate well. Thus, the chimera may have limited replication ability, cause no disease, but still elicit immunity to the surface envelope proteins of HCV and thus have potential as a hepatitis C vaccine. These chimeras can be tested for their ability to promote immunity to HCV through an immune response.

### Example 8

With regard to chimeric genomes derived from the HCV and comprising the sequences coding E1-E2-p13-NS2-NS3ₚᵣₒ or C-E1-E2-p13-NS2-NS3pro of GBV-B instead of E1-E2-p7-NS2 sequences or C-E1-E2-p7-NS2 of the HCV, which had given encouraging results, the nucleotide sequence of both chimeric cDNAs HC/E1-NS3ₚᵣₒGB-Ubi and HC/C-NS3ₚᵣₒGB-Ubi was confirmed. Indeed, the plasmids containing these cDNA are very low copy number and it is difficult to propagate them in great quantity in the bacteria. In order to avoid any risk of appearance of specific changes during the amplification of the plasmids in the bacterium, the inventors sequenced the plasmid preparation used for in vitro transcription studies. Instead of working with two independent clones for each construction (the initial strategy to minimize the risks of undesirable change in a molecular clone), one validated clone was used and thus limit the quantities of RNA inoculated to the animal. Once the sequences of the two cDNA were confirmed relative to the approximately 10250 bases, RNA was prepared by in vitro transcription using the RNA polymerase of the phage T7, whose promoter is cloned upstream of the target cDNA.

The resulting RNA, whose quality and quantity were controlled, were sent to the laboratory of Dr. Lanford (Southwest Foundation for Biomedical Research, San Antonio, Tex.) for inoculation in the liver of two tamarins (T16472, T16467). These two animals had been inoculated (4 and 2 years before, respectively) with other chimeric genomes derived from GBV-B, with no sign of viremia being detected during the 22 week observation period. The inventors chose to use two animals in order to optimize the chances to select changes “adapatation” conferring an effective replication at this experimental host. Such changes are probably necessary to obtaining a chimeric virus with robust replicative capacities, with respect to the results of the first inoculations of these chimeric genomes. There was no trace of GBV-B sequences in the serum of the animals taken one week before the inoculation. A mixture of approximately 100 μg of each RNA HC/E1-NS3ₚᵣₒGB-Ubi and HC/C-NS3ₚᵣₒGB-Ubi was then inoculated intrahepatically. The quality of the inoculated RNA was checked the day of the inoculation by agarose gel. Samples of serum were prepared to 0, 1, 2 days, then every two weeks after the inoculation and were tested by specific quantitative RT-PCR for the genome of the HCV (primers and probes typically hybridized in the 5′ non-coding region of the genome) to seek the presence of viral particles. Assays to detect the rise in serum transaminases allows characterization of possible hepatitis on the biochemical level. The serums from 0 to 16 weeks post-inoculation were tested. The results obtained to date seem to indicate that a viremy (2×10⁴ genome equivalent/ml to the maximum) could be detected at 8 to 12 weeks post-inoculation in the two animals, but no signal was then detected in 14 and 16 weeks post-inoculation.

The inventors will pass these inoculums to naive tamarins and examine whether one can thus force the adaptation of such a chimeric virus. With regard to the chimeric genomes derived from GBV-B and comprising the sequences coding E1-E2-p7-NS2-NS3ₚᵣₒ or C-E1-E2-p7-NS2-NS3ₚᵣₒ of the HCV instead of E1-E2-p13-NS2 sequences or C-E1-E2-p13-NS2 of GBV-B, cDNA GB/E1-NS3ₚᵣₒ^{HC}-Ubi and GB/C-NS3ₚᵣₒ^{HC}-Ubi nucleotide substitution allowing the transition from the residue Val(2236) of NS5A (coded by the clone) to the Ala residue, whose codon is systematically found in the genome of all the viruses resulting from our molecular clone, and who thus confers a replicative advantage on the virus. The nucleotide sequence of both new cDNA construct also was sequenced in its entirety, in order to exclude any potential undesirable change which could have appeared during amplifications of plasmids in the bacterium. Repair of the plasmid GB/C-NS3ₚᵣₒ^{HC}-Ubi was carried out by exchange of a restriction fragment containing the parental residue, then the sequence of cDNA of the new clone was confirmed. A mixture of the two chimeric RNA derived from these modified GBV-B clones were inoculated intrahepatically in two animals to test their infectivity. The inventors envision the use of two other animals for the evaluation of other chimeric genomes targeting non-structural proteins and the effects on replication of the viral RNA. They are genomes derived from GBV-B and having the sequences from the protein p7 HCV instead of whole or part from the protein p13 from GBV-B. The protein p7 of the HCV has an ion channel function (Pavlovic et al, 2003, Proc Natl Acad Sci USA 100:6104; Premkumar et al., 2004, FEBS Lett 557:99), potentially implied in the stages of synthesis and/or export of the virions. The existence of a similar protein for GBV-B (p13) has been shown, but of double size, composed of two parts equipped with potentially distinct functions (Ghibaudo et al., 2004, J Biol Chem 279:24965). A cDNA for the sequences coding p13 or coding the final half C of p13 (homologous with p7) were substituted by the sequence of p7 of the HCV. The chimeric viruses having such genomes would constitute invaluable tools, as well for the study of the role of these candidates in the infectious cycle of the hepacivirus, as for research the new antiviral ones targeting p7 HCV and being able to interfere with the formation of the infectious particles. Such research is difficult with HCV, whose only experimental host is the chimpanzee.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents, which are both chemically and physiologically related, may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,578,770
U.S. Pat. No. 4,596,792
U.S. Pat. No. 4,599,230
U.S. Pat. No. 4,599,231
U.S. Pat. No. 4,601,903
U.S. Pat. No. 4,608,251
U.S. Pat. No. 4,683,195
U.S. Pat. No. 4,683,202
U.S. Pat. No. 4,800,159
U.S. Pat. No. 4,883,750
U.S. Pat. No. 5,279,721
U.S. Pat. No. 5,807,670
EPA No. 320,308
EPA No. 329,822
GB Application No. 2,202,328
PCT/US87/00880
PCT/US89/01025
WO 88/10315
WO 89/06700
WO 90/07641
Agapov et al., J. Virol, 78:2414-2425, 2004.
Al et al., Virus Res., 53:141-149, 1998.
Alter et al., Hepatology, 26:625-655, 1997.
Baumert et al. J. Virol., 72:3827-3836, 1998.
Beames et al., J. Virol., 74:11764-11772, 2000.
Beard et al., Hepatology, 1999.
Behrens et al., EMBO. J, 15:12-22, 1996.
Carrere-Kremer et al., J. Virol., 76:3720-3730, 2002.
Chambers et al., J. Virol., 73:3095-3101, 1999.
Chambers et al., J. Virol., 73:3095-3101, 1999.
Choo et al., Proc. Natl. Acad. Sci. USA, 91:1294-1298, 1994.
Consensus Development Panel. National Institutes of Health Consensus Development Conference Panel statement: Management of hepatitis C. Hepatology, 26:Supplement 1[3], 2S-10S, 1997.
Dubuisson, Curr. Top. Microbiol. Immunol., 242:135-148, 2000.
Farci et al., J. Infectious Diseases, 165:1006-1011, 1992.
Farci et al., Science, 258:135-140, 1992.
Filocamo et al., J. Virol., 71 1417-27, 1997.
Frohman, PCR Protocols: A Guide To Methods And Applications, Academic Press, New York, 1990.
Frolov et al., RNA, 4:1418-1435, 1998.
Ghibaudo et al., J. Biol. Chem., 279:24965-24975, 2004.
Ghibaudo et al., J. Biol. Chem., 279:24965-24975, 2004.
Grakoui et al., J. Virol., 67(3):1385-95, 1993.
Grakoui et al., J. Virol., 67(5):2832-2843, 1993.
Grakoui et al., Proc. Natl. Acad. Sci. USA, 90:10583-10587, 1993.
Griffin et al., FEBS Lett., 535:34-38, 2003.
Hijikata et al., J. Virol., 67:4665-4675, 1993.
Honda et al., RNA, 2:955-956, 1996.
Inchauspe et al., Proc. Natl. Acad. Sci. USA, 88:10292-10296, 1991.
Karayiannis et al., Hepatology, 9:186-192, 1989.
Kim et al., Cell, 87:343-355, 1996.
Kolykhalov et al., J. Virol., 70:3363-3371, 1996.
Kolykhalov et al., Science, 277:570-574, 1997.
Kummerer and Rice, J. Virol., 76:4773-4784, 2002.
Landt et al., Gene, 96:125-128, 1990.
Lemon and Honda, Seminars in Virology, 8:274-288, 1997.
Lindenbach and Rice, J. Virol., 73:4611-4621, 1999.
Lo et al., J. Virol., 70:5177-5182, 1996.
Lohmann et al., Science, 285:110-113, 1999.
Lohmann et al., Science, 285:110-3, 1999.
Lu and Wimmer, Proc., Natl., Acad., Sci., USA, 93:1412-1417, 1996.
Martin et al., Proc. Natl. Acad. Sci. USA, 100:9962-9967, 2003.
McLauchlan et al., EMBO J., 21:3980-3988, 2002.
McLauchlan et al., EMBO J., 21:3980-3988, 2002.
Muerhoff et al., J. Virol., 69:5621-5630, 1995.
Neumann et al., Science, 282:103-107, 1998.
Op De Beeck et al., J. Gen. Virol., 82:2589-2595, 2001.
Pieroni et al., J. Virol., 71:6373-6380, 1997.
Pletnev and Men, Proc. Natl. Acad. Sci. USA, 95:1746-1751, 1998.
Pletnev et al., Proc. Natl. Acad. Sci. USA, 89:10532-10536, 1992.
Remington's Pharmaceutical Sciences, 15^{th} Edition, pages 1035-1038 and 1570-1580.
Reynolds et al., EMBO. J., 14:6010-6020, 1995.
Rijnbrand et al., Mutational analysis of the GB virus B internal ribosome entry site. Submitted for publication, 1999.
Rijnbrand et al., RNA, 7:585-597, 2001.
Sakai et al., Proc. Natl. Acad. Sci. USA, 100:11646-11651, 2003.
Sambrook, et al., Molecular Cloning, A Laboratory Manual, 2nd ed., Cold Spring Harbor Laboratory, Cold Spring, Harbor, N.Y., 1989.
Scarselli et al., J. Virol., 71 4985-4989, 1997.
Schlauder et al., J. Med. Virol., 46:81-90, 1995.
Shaffer et al., J. Virol., 69:600-6604, 1995.
Simons, et al., Proc. Natl. Acad. Sci., 92:3401-3405, 1995.
Tanaka et al., Biochem. Biophys. Res. Comm., 215:744-749, 1995.
Teller et al., J. Clin. Micro., 34:3085-3091, 1996.
Thibeault et al., J. Biol. Chem., 276:46678-46684, 2001.
Todd et al., J. Virol., 71:8868-74, 1997.
Yamaga and Ou, Biol. Chem., 277:33228-33234, 2002.
Yanagi et al., Proc. Natl. Acad. Sci. USA, 16:8738-8743, 1998.
Yanagi et al., Proc. Natl. Acad. Sci. USA, 94:8738-8743, 1997.
Yao et al., Nature Structural Biology, 4: 463-467, 1997.
Yasui et al., J. Virol., 72:6048-6055, 1998.
Yi and Lemon, J. Virol., 78:7904-7915, 2004.
Zhao et al., J. Virol., 73:1546-1554, 1999.

What is claimed is:

1. A chimeric hepatotropic recombinant virus comprising part of a GBV-B polynucleotide and part of the polynucleotide sequence derived from HCV.

2. The hepatotropic virus of claim 1, wherein the nucleic acid segment of HCV encodes a core protein.

3. The hepatotropic virus of claim 1, wherein the nucleic acid segment of HCV encodes an E1 protein.

4. The hepatotropic virus of claim 3, wherein the nucleic acid segment of HCV encodes an E2 protein.

5. The hepatotropic virus of claim 1, wherein the nucleic acid segment of HCV encodes a p7 protein.

6. The hepatotropic virus of claim 1, wherein the nucleic acid segment of HCV encodes an E1 and E2 protein.

7. The hepatotropic virus of claim 1, wherein the nucleic acid segment of HCV encodes a core, E1, and E2 proteins.

8. The hepatotropic virus of claim 1, wherein the nucleic acid segment of HCV encodes a core, E1, E2, and p7 proteins.

9. The hepatotropic virus of claim 1 further comprising a NS2 protein.

10. The hepatotropic virus of claim 1, further comprising a NS3 protein having a heterologous protease cleavage site.

11. The hepatropic virus according to claim 1 further comprising a pair of NS3 having a heterologous protease cleavage site.

12. The hepatotropic virus of claim 1, wherein the heterologous cleavage site is an ubiquitin protease cleavage site.

13. The hepatotropic virus of claim 1, wherein the polynucleotide comprises at least 1% of an HCV genome.

14. The hepatotropic virus of claim 1, wherein the polynucleotide comprises at least 5% of an HCV genome.

15. The hepatotropic virus of claim 1, wherein the polynucleotide comprises at least 10% of an HCV genome.

16. The hepatotropic virus of claim 1, wherein the polynucleotide comprises at least 20% of an HCV genome.

17. The hepatotropic virus of claim 1, wherein the polynucleotide comprises at least 30% of an HCV genome.

18. The hepatotropic virus of claim 1, wherein the polynucleotide comprises at least 40% of an HCV genome.

19. The hepatotropic virus of claim 1, wherein the polynucleotide comprises at least 50% of an HCV genome.

20. The hepatotropic virus of claim 1, wherein the polynucleotide comprises at least 60% of an HCV genome.

21. The hepatotropic virus of claim 1, wherein the polynucleotide comprises at least 70% of an HCV genome.

22. The hepatotropic virus of claim 1, wherein the polynucleotide comprises at least 80% of an HCV genome.

23. The hepatotropic virus of claim 1, wherein the polynucleotide comprises at least 90% of an HCV genome.

24. The hepatotropic virus of claim 1, wherein the polynucleotide comprises at least 95% of an HCV genome.

25. The hepatotropic virus of claim 1, wherein the polynucleotide has a sequence set forth in SEQ ID NO:17, SEQ ID NO:18, SEQ ID NO:19, SEQ ID NO:20, SEQ ID NO:21, SEQ ID NO:22, SEQ ID NO:24, or SEQ ID NO:26.

26. The hepatotropic virus of claim 1, wherein the virus propagates in vivo.

27. A method of producing a chimeric virus comprising:
a) introducing into a host cell an expression construct comprising a polynucleotide encoding a chimeric GBV-B/HCV virus; and
b) culturing said host cell under conditions permitting production of a chimeric virus from the construct.

28. The method of claim 27, wherein the host cell is a prokaryotic cell.

29. The method of claim 27, wherein the host cell is a eukaryotic cell.

30. The method of claim 29, wherein the host cell is in an animal.

31. The method of claim 30, wherein the host cell is in a tamarin.

32. The method of claim 27, wherein the polynucleotide comprises synthetic RNA.

33. The method of claim 27, wherein the polynucleotide comprises synthetic DNA.

34. The method of claim 27, further comprising the step of isolating virus from the host cell.

35. The method of claim 34, wherein said virus is purified to homogeneity.

36. A method for identifying a compound active against a viral infection comprising:
a) providing an expression construct comprising a polynucleotide that when expressed produces a chimeric GBV-B/HCV virus;
b) contacting the virus with a candidate substance; and
c) comparing the infectious ability of the virus in the presence of the candidate substance with the infectious ability of the virus in a similar system in the absence of the candidate substance.

37. A method of producing a virus comprising:
a) introducing into a host cell an expression construct comprising a chimeric GBV-B polynucleotide encoding at least part of an HCV sequence; and
b) culturing said host cell under conditions permitting production of a virus from the construct.

38. The method of claim 37, wherein said host cell is a eukaryotic cell.

39. The method of claim 38, wherein said host cell is in an animal.

40. The method of claim 37, wherein said polynucleotide comprises synthetic RNA.

41. The method of claim 37, further comprising the step of isolating virus from said host cell.

42. The method of claim 41, wherein said virus is purified to homogeneity.

43. A compound active against a viral infection identified according to a method comprising:
a) providing a virus expressed from an construct comprising GBV-B/HCV chimera;
b) contacting the virus with a candidate substance; and
c) comparing the infectious ability of the virus in the presence of the candidate substance with the infectious ability of the virus in a similar system in the absence of the candidate substance.

44. The polynucleotide of claim 1 wherein the polynucleotide as a set forth in SEQ ID 23, SEQ ID 24, SEQ ID 25, SEQ ID 19, SEQ ID 20, SEQ ID 21, SEQ ID 22, SEQ ID 24, or SEQ ID 126.

45. The polynucleotide of claim 1 wherein the HCV or GVB-B nucleotide sequences comprise at least one of the sequences of SEQ ID 23, SEQ ID 24, SEQ ID 25, SEQ ID 19, SEQ ID 20, SEQ ID 21, SEQ ID 22, SEQ ID 24, or SEQ ID 26 or a fragment thereof which said fragment leads to a recombinant chimeric virus of claim 1.
